(19) United States
(12) Patent Application Publication
(10) Pub. No.: US 2006/0241082 A1
(43) Pub. Date: Oct. 26, 2006

(54) Modulating vesicular monoamine transporter trafficking and function: a novel approach for the treatment of parkinson's disease

(76) Inventors: Annette E Fleckenstein, Salt Lake City, UT (US); Glen R. Hanson, (US)

Correspondence Address: NEEDLE & ROSENBERG, P.C., SUITE 1000, 999 PEACHTREE STREET, ATLANTA, GA, 30309-3915, US

(21) Appl. No.: 10/528,684

(22) PCT Filed: Sep. 19, 2003
(86) PCT No.: PCT/US03/29668

§ 371 Date: May 9, 2005

Related U.S. Application Data
(60) Provisional application No. 60/412,439, filed on Sep. 19, 2002.

(51) Int. Cl. A61K 31/675; A61K 31/54; A61K 31/537; A61K 31/445; A61K 31/495; A61K 31/428

(52) U.S. Cl. 514/89; 514/317; 514/367; 514/227.5; 514/237.5; 514/252.12

(57) ABSTRACT

Disclosed are compositions and methods for treating Parkinson's disease.

### CROSS REFERENCE TO RELATED APPLICATIONS

This application claims the benefit of U.S. provisional application Ser. No. 60/412,439, filed Sep. 19, 2002. This application is hereby incorporated by this reference in its entirety for all of its teachings.

### ACKNOWLEDGEMENTS

This invention was made with government support under federal grants DA04222, DA00869, DA11389, and DA013367, DA13367, and DA14475.awarded by the NIDA and NIH. The Government has certain rights to this invention.

### BACKGROUND

Parkinson's disease is a neurodegenerative disease. While the much research has gone into finding therapeutics which can reduce inhibit the effects of Parkinson's, little has been accomplished in preventing or inhibiting the causative mechanisms of the disease. Disclosed are compositions and methods which can reduce and inhibit the underlying neurodegeneration that causes the effects of Parkinson's disease to be so devastating.

### SUMMARY

Disclosed are methods and compositions related to treating Parkinson's disease.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the results of an experiment in which rats which received a single administration of saline vehicle (1 ml/kg, s.c.) or METH (5, 10 or 15 mg/kg; s.c.) and were decapitated 1 h later. Uptake was determined using a single concentration of [³H]DA as described in Materials and Methods. Symbols represent the means and vertical lines 1 S.E.M. of determinations in 6 rats. *Value for treated rats that is significantly different from saline-treated controls (p≦0.05).

FIG. 2 shows the results of an experiment in which rats received a single administration of METH (15 mg/kg, s.c.) and were decapitated 1 or 24 h later. Other rats received saline vehicle (1 ml/kg, s.c.) and were decapitated 1 h later. Symbols represent the means and vertical lines 1 S.E.M. of determinations in 6 rats. *Value for treated rats that is significantly different from saline-treated controls (p≦0.05).

FIG. 3 shows the results of an experiment in which rats received a single administration of SCH23390 (0.5 mg/kg, i.p.) or saline vehicle (1 ml/kg i.p.) 15 min prior to a single administration of either saline vehicle (1 ml/kg, s.c.) or METH (15 mg/kg, s.c.). All rats were decapitated 1 h following the last drug injection. Columns represent the means and vertical lines 1 S.E.M. of determinations in 6 rats. *Values for treated rats that are significantly different from saline-treated controls (p≦0.05).

FIG. 4 shows the results of an experiment in which rats received a single administration of eticlopride (0.5 mg/kg, i.p) or saline vehicle (1 ml/kg) 15 min prior to a single administration of either METH (15 mg/kg, s.c.) or saline vehicle (1 ml/kg, s.c.). All rats were maintained in an ambient environment of 24° C., except where indicated where rats were place in a 28.5° C. environment (see Methods). All animals were decapitated 1 h following the last drug injection. Columns represent the means vertical lines represent 1 S.E.M. of determinations in 6 rats. *Value for treated rats that is significantly different from saline-treated controls (p≦0.05).

FIG. 5 shows the results of an experiment in which rats received either a single administration of METH (15 mg/kg, s.c.) or saline vehicle (1 ml/kg, s.c.), and were decapitated 1 h later. All rats were maintained in an ambient environment of 24° C., except where indicated where rats were place in a 6° C. environment (see Methods). Columns represent the means and vertical lines represent 1 S.E.M. of determinations in 6 rats. *Values for treated rats that are significantly different from saline-treated controls (p≦0.05).

FIG. 6 shows the results of an experiment in which rats received a single administration of quinpirole (1 mg/kg, i.p) or saline vehicle (1 ml/kg) immediately prior to a single administration of either METH (15 mg/kg, s.c.) or saline vehicle (1 ml/kg, s.c.). All animals were decapitated 1 h following the last drug injection. Columns represent the means vertical lines represent 1 S.E.M. of determinations in 6 rats. *Values for treated rats that are significantly different from saline-treated controls (p≦0.05).

FIG. 7 shows the results of an experiment in which rats received a single administration of cocaine (30 mg/kg, i.p) or saline vehicle (1 ml/kg) immediately prior to a single administration of either METH (15 mg/kg, s.c.) or saline vehicle (1 ml/kg, s.c.). All animals were decapitated 1 h following the last drug injection. Columns represent the means vertical lines represent 1 S.E.M. of determinations in 7-13 rats. *Values for treated rats that are significantly different from saline-treated controls (p≦0.05).

FIG. 8 shows the time-response effect of multiple MDMA administrations on striatal plasmalemmal [³H]DA uptake and [³H]WIN35428 binding. Rats received four injections (2-h intervals) of MDMA (10 mg/kg/injection, s.c.) or saline vehicle (1 ml/kg/injection, s.c.). Rats were decapitated 1 or 24 h after the final injection. Symbols represent the means and vertical lines 1 SEM of determinations in six to eight rats. *Values for MDMA-treated rats that are significantly different from saline-treated controls (p≦0.05).

FIG. 9A shows the effect of core body temperature on the decrease in striatal plasmalemmal [³H]DA uptake caused by multiple administrations of MDMA. Rats were maintained in an ambient temperature of 24° C. before treatment. Upon receiving MDMA (4×10 mg/kg, s.c.; 2-h intervals) or saline (1 ml/kg, s.c.; 2-h intervals), rats were exposed to 6 or 24° C. ambient temperature for the duration of the experiment. Rats were decapitated 1 h after the last MDMA or saline administration. Panel B: Time course of core body temperatures. Downward arrows represent time points of MDMA or saline administration. *Values for MDMA-treated rats that are significantly different from saline-treated controls (p≦0.05).

FIG. 10 shows the effect of αMT on the decrease in striatal plasmalemmal [³H]DA uptake (panel A) and [³H]WIN35428 binding (panel B) caused by multiple administrations of MDMA. αMT (150 mg/kg, i.p.) was administered 5 and 1 h before to and 3 h after the first injection of MDMA. Rats received four injections (2-h intervals) of MDMA (10 mg/kg/injection, s.c.) or saline vehicle (1 ml/kg/injection, s.c.) and were decapitated 1 h later. Columns represent the means and vertical lines 1 SEM of determinations in six to eight rats. *Values for MDMA-treated rats that are significantly different from saline-treated controls (p≦0.05).

FIG. 11 shows the effects of NPC15437 (panel A) and Ro31-7549 (panel B) pretreatment on the decrease in plasmalemmal [³H]DA uptake in striatal synaptosomes induced by MDMA preincubation. Striatal synaptosomes were pretreated with 10 μM NPC15437 or 10 μM Ro31-8220 for 5 min and subsequently exposed to 10 μM MDMA or assay buffer for 30 min at 37° C. *Values for MDMA-treated preparations that are significantly different from saline-treated controls (p≦0.05).

FIG. 12 shows a time-response effect of multiple MDMA administrations on striatal vesicular [³H]DA uptake and [³H]DHTBZ binding. Rats received four injections (2-h intervals) of MDMA (10 mg/kg/injection, s.c.) or saline vehicle (1 ml/kg/injection, s.c.). Rats were decapitated 1 or 24 h after the final injection. Symbols represent the means and vertical lines 1 SEM of determinations in six to eight rats. *Values for MDMA-treated rats that are significantly different from saline-treated controls (p≦0.05).

FIG. 13 shows the effect of core body temperature on the decrease in striatal vesicular [³H]DA uptake panel A) and [³H]DHTBZ binding caused by multiple administrations of MDMA. Rats were maintained in an ambient temperature of 24° C. before treatment. Upon receiving MDMA (4×10 mg/kg, s.c.; 2-h intervals) or saline (1 ml/kg, s.c.; 2-h intervals), rats were exposed to 6 or 24° C. ambient temperature for the duration of the experiment. Rats were decapitated 1 h after the last MDMA or saline administration. Panel C: Time course of core body temperatures. Downward arrows represent time points of MDMA or saline administration. *Values for MDMA-treated rats that are significantly different from saline-treated controls (p≦0.05).

FIG. 14 shows the effect of eticlopride on the decrease in striatal vesicular [³H]DA uptake caused by multiple administrations of MDMA. Eticlopride (0.5 mg/kg, i.p.) or saline vehicle (1 ml/kg, i.p.) was administered 15 min before each MDMA injection. Rats received four injections (2-h intervals) of MDMA (10 mg/kg/injection, s.c.) or saline vehicle (1 ml/kg/injection, s.c.) and were decapitated 1 h later. Columns represent the means and vertical lines 1 SEM of determinations in six to eight rats. *Values for MDMA-treated rats that are significantly different from saline-treated controls (p≦0.05).

FIG. 15 shows the results of an experiment in which treated mice received 4 injections of methamphetamine (10 mg/kg/injection, s.c., 2-h intervals) and were sacrificed 1 or 24 h later. Control mice received 4 injections of saline vehicle (5 ml/kg/injection, s.c.) and were sacrificed 1 h later (zero-time controls). Filled circles and squares represent mean vesicular dopamine uptake and DHTBZ binding, respectively, and vertical lines 1 S.E.M. of determinations in 6 mice. *Values significantly different from zero-time controls (p≦0.05).

FIG. 16 shows the results of an experiment in which mice received 4 injections of methamphetamine (METH; 10 mg/kg/injection, s.c., 2-h intervals) or saline vehicle (5 ml/kg/injection, s.c.) and were sacrificed 1 h later. Columns represent means and vertical lines 1 S.E.M. of determinations in 4 mice. *Value significantly different from saline-treated controls (p≦0.05).

FIG. 17 shows the results of an experiment in which mice received 4 injections of methamphetamine (10 mg/kg/injection, s.c., 2-h intervals) or saline vehicle (5 ml/kg/injection, s.c.) and were sacrificed 1 h later. In addition, mice received SCH23390 (2 mg/kg, i.p.) or saline vehicle (5 ml/kg, i.p.) min prior to each injection of methamphetamine or saline vehicle. Columns represent means and vertical lines 1 S.E.M. of determinations in 6 mice. *Value significantly different from saline-treated controls (p≦0.05).

FIG. 18 shows the results of an experiment in which mice received 4 injections of methamphetamine (10 mg/kg/injection, s.c., 2-h intervals) or saline vehicle (5 ml/kg/injection, s.c.) and were sacrificed 1 h later. In addition, mice received eticlopride (2 mg/kg, i.p.) or saline vehicle (5 ml/kg, i.p.) min prior to each injection of methamphetamine or saline vehicle. Columns represent means and vertical lines 1 S.E.M. of determinations in 6 mice (Upper panel). *Values significantly different from saline-treated controls (p≦0.05). #Value significantly different from rats receiving methamphetamine per se in a 23° C. environment. Corresponding body temperatures are presented in the lower panel.

FIG. 19 shows the results of an experiment in which treated mice received 4 injections of MDMA (10 mg/kg/injection, s.c., 2-h intervals) and were sacrificed 1 or 24 h later. Control mice received 4 injections of saline vehicle (5 ml/kg/injection, s.c.) and were sacrificed 1 h later (zero-time controls). Filled circles and squares represent mean vesicular dopamine uptake and DHTBZ binding, respectively, and vertical lines 1 S.E.M. of determinations in 6 mice. *Values significantly different from zero-time controls (p≦0.05).

FIG. 20 shows the results of an experiment in which mice received a single injection of methylphenidate (50 mg/kg, s.c.), cocaine (30 mg/kg, i.p.) or saline vehicle (5 ml/kg s.c.) and were sacrifice 1 h later. Columns represent means and vertical lines 1 S.E.M. of determinations in 6 mice. *Values significantly different from saline-treated controls (p≦0.05).

FIG. 21 shows that cocaine alters VMAT-2 immunoreactivity in subcellular fractions. Rats received a single administration of cocaine (30 mg/kg, i.p.) or saline vehicle (1 ml/kg, s.c.). All animals were sacrificed 1 h after the cocaine or saline injection. Columns represent the mean optic density, and error bars represent the S.E.M. of determinations in six treated rats. *Values for cocaine-treated rats that are significantly different from saline-treated controls (p≦0.05).

FIG. 22 shows methamphetamine alters VMAT-2 immunoreactivity in subcellular fractions. Rats received multiple high-dose injections of methamphetamine (4×10 mg/kg per injection, s.c., 2-h intervals), or saline vehicle (1 ml/kg per injection). All animals were sacrificed 1 h after the final methamphetamine or saline injection. Columns represent the mean optic density, and error bars represent the S.E.M. of determinations in six treated rats. *Values for methamphetamine-treated rats that are significantly different from saline-treated controls (p≦0.05).

FIG. 23 shows that a single administration of MPD increases vesicular [³H]DA uptake and [³H]DHTBZ binding. Rats received a single administration of MPD (5-40 mg/kg, s.c.) or saline vehicle (1 ml/kg, s.c.) and were sacrificed 1 h later. Symbols represent the means and vertical lines 1 S.E.M. of determinations in six rats. Data are expressed as a percentage of the mean of control. Mean control values for vesicular [³H]DA uptake and [³H]DHTBZ binding ranged from 81.4 to 167.3 fmol/μg protein and 1.2 to 2.3 fmol/μg protein, respectively. *Values for MPD-treated rats that are significantly different from saline-treated controls (p≦0.05).

FIG. 24 shows that a single administration of MPD rapidly and reversibly increases vesicular [³H]DA uptake and [³H]DHTBZ binding. Rats received a single administration of MPD (5, 10 or 40 mg/kg, s.c.) or saline vehicle (1 ml/kg, s.c.) and were sacrificed 30 min to 12 h later. Symbols represent the means and vertical lines 1 S.E.M. of determinations in six rats. Data are expressed as a percentage of the mean of control. Mean control values for vesicular [³H]DA uptake and [³H]DHTBZ binding ranged from 135.2 to 226.3 fmol/μg protein and 4.6 to 7.1 fmol/μg protein, respectively. *Values for MPD-treated rats that are significantly different from saline-treated controls (p≦0.05).

FIG. 25 shows that a single administration of MPD increases the Vₘₐₓ of vesicular [³H]DA uptake. Rats received a single administration of MPD (40 mg/kg, s.c.) or saline vehicle (1 ml/kg, s.c.) and were sacrificed 1 h later. The Eadie-Hofstee plot depicts data from one of four experiments, with samples in each run in duplicate. The mean Kₘ values were 235±27 and 230±10 nM for saline- and MPD-treated rats, respectively. The mean Vₘₐₓ values for all four experiments combined were 1584±129 and 2350±250 fmol/μg protein/3 min for saline- and MPD-treated rats, respectively; these values differed significantly (p≦0.05).

FIG. 26 shows that a single administration of MPD redistributes VMAT-2 immunoreactivity. Rats received a single administration of MPD (40 mg/kg, s.c.) or saline vehicle (1 ml/kg, s.c.). All animals were sacrificed 1 h after the MPD or saline injection. Columns represent the mean optic density, and error bars represent the S.E.M. of determinations in six treated rats. Molecular mass standards (in kD) are shown to the left of the representative Western blot. *Values for MPD-treated rats that are significantly different from saline-treated controls (p≦0.05).

FIG. 27 shows that a DA D₁ receptor antagonist, SCH23390, attenuates the MPD-induced increases in vesicular [³H]DA uptake, [³H]DHTBZ binding and VMAT-2 immunoreactivity. Rats received a single administration of SCH23390 (SCH; 0.5 mg/kg, i.p.) or saline vehicle (1 ml/kg, i.p.) 15 min prior to a single administration of either MPD (40 mg/kg, s.c.) or saline vehicle (1 m/kg, s.c.). All animals were sacrificed 1 h after the last injection. Columns represent the mean vesicular [³H]DA uptake and [³H]DHTBZ binding, and error bars represent the S.E.M. of determinations in six treated rats. Molecular mass standards (in kD) are shown to the left of the representative Western blot. *Values for MPD-treated rats that are significantly different from saline-treated controls; #values for SCH/MPD-treated animals that are significantly different from MPD-treated animals (p≦0.05).

FIG. 28 shows that a DA D₂ receptor antagonist eticlopride, attenuates the MPD-induced increases in vesicular [³H]DA uptake, [³H]DHTBZ binding and VMAT-2 immunoreactivity. Rats received a single administration of eticlopride (Etic; 0.5 mg/kg, i.p.) or saline vehicle (1 ml/kg, i.p.) 15 min prior to a single administration of either MPD (40 mg/kg, s.c.) or saline vehicle (1 ml/kg, s.c.). All animals were sacrificed 1 h after the last injection. Columns represent the mean vesicular [³H]DA uptake and [³H]DHTBZ binding, and error bars represent the S.E.M. of determinations in six treated rats. Molecular mass standards (in kD) are shown to the left of the representative Western blot. *Values for MPD-treated rats that are significantly different from saline-treated controls; #values for Etic/MPD-treated animals that are significantly different from MPD-treated animals (p≦0.05).

FIG. 29 shows that coadministration of SCH23390 and eticlopride blocks the MPD-induced increases in vesicular [³]DA uptake and [³H]DHTBZ binding. Rats received a single administration of SCH23390 and eticlopride (SCH & Etic; 0.5 mg/kg, i.p.) or saline vehicle (1 ml/kg, i.p.) 15 min prior to a single administration of either MPD (40 mg/kg, s.c.) or saline vehicle (1 ml/kg, s.c.). All animals were sacrificed 1 h after the last injection. Columns represent the mean vesicular [³H]DA uptake and [³H]DHTBZ binding, and error bars represent the S.E.M. of determinations in six treated rats. *Values for MPD-treated rats that are significantly different from saline-treated controls (p≦0.05).

FIG. 30 shows that multiple administrations of METH decrease VMAT-2 immunoreactivity. Rats received METH (4 injections; 7.5 mg/kg; s.c.; 2-h intervals) or saline (1 ml/kg; s.c.). All animals were sacrificed 1 h after the last METH or saline injection. VMAT-2 immunoreactivity was assessed in a whole syanptosomal fraction (P2), a plasmalemmal membrane fraction (P3) and a vesicular subcellular fraction (S3). Columns represent the mean band density, and error bars represent the S.E.M. of determinations in six treated rats. *Values for MPD-treated rats that are significantly different from saline-treated controls (p≦0.05).

FIG. 31 shows that post-treatment with MPD attenuates the METH-induced dopaminergic deficits. FIG. 31A. Rats received METH (4 injections; 7.5 mg/kg; s.c.; 2-h intervals) or saline (1 ml/kg; s.c.). Rats received one injection of MPD (5 mg/kg; s.c.), 2 injections of MPD, 3 injections of MPD or saline (sal; 1 ml/kg; s.c.) after the last METH or saline injection. Rats were sacrificed 1 h after the last METH or saline administration. Columns represent the means and vertical lines 1 S.E.M. of determinations in 8-12 rats. *Values that are significantly different from Sal/Sal-treated group. FIG. 31B. Time-course of core body temperatures. Rectal temperatures were recorded prior to the first MPD, METH or saline injection (t=0 h), and every hour thereafter (t=0-7 h). Gray inverted arrows represent time-points of MPD or saline administrations and black inverted arrows represent time-points of METH or saline administrations. Symbols represent means, and vertical lines 1 S.E.M. of determinations in 8-12 rats. *Values that are different from Sal/Sal-treated group (p≦0.05).

FIG. 32 shows that post-treatment with MPD attenuates the acute METH-induced decrease in striatal vesicular DA uptake and DHTBZ binding. Rats received METH (4 injections; 7.5 mg/kg; s.c.; 2-h intervals) or saline (1 ml/kg; s.c.). Rats received one injection of MPD (5 mg/kg; s.c.), 2 injections of MPD, 3 injections of MPD or saline (sal; 1 ml/kg; s.c.) after the last METH or saline injection. Rats were sacrificed 1 h after the last METH or saline administration. Columns represent the means and vertical lines 1 S.E.M. of determinations in 6 rats. *Values that are significantly different from the Sal/Sal-treated group; #values that are significantly different from the METH/Sal-treated group (p≦0.05).

FIG. 33 shows that multiple administrations of METH decreased vesicular DA uptake and DHTBZ binding. Rats received METH (4 injections; 7.5 mg/kg; s.c.; 2-h intervals) or saline (1 ml/kg; s.c.). Rats were sacrificed 1 h, 2 h, 4 h, or 6 h after the last METH or saline administration. Symbols represent the means and vertical lines 1 S.E.M. of determinations in 6 rats. *Values that are significantly different from the Sal/Sal-treated group.

FIG. 34 shows that post-treatment with MPD does not alter total striatal tissue DA content (FIG. 34 A), but attenuates the METH-induced decrease in vesicular DA content (FIG. 34B). Rats received METH (4 injections of 7.5 mg/kg; s.c.; 2-h intervals) or saline (1 ml/kg; s.c.). Rats received 3 injections of MPD (5 mg/kg; s.c.) or saline (sal; 1 ml/kg; s.c.) after the last METH or saline injection. Rats were sacrificed 1 h after the last METH or saline administration. Columns represent the means and vertical lines 1 S.E.M. of determinations in 6 rats. *Values that are significantly different from Sal/Sal-treated groups (p≦0.05).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

Abbreviations: VMAT-2, vesicular monoamine transporter-2; DA, dopamine; DHTBZ, dihydrotetrabenazine; METH, methamphetamine, DAT, dopamine transporter, D1, dopamine receptor 1, D2, dopamine receptor 2.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Treating” does not mean a comlete cure. It means that the symptoms of the underlying disease are reduced, and/or that the underlying cellular mechanisms causing the symptoms are reduced. It is understood that reduced, as used in this context, means relative to the state of the disease, including the molecular state of the disease, not just the physiological state of the disease.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Variables such as R¹-R¹⁴, A, Q, U, V, X, Y, E, G, J, L, m, n, p, q, s, carbons a and b, and bonds d and e used throughout the application are the same variables as previously defined unless defined to the contrary.

The term “alkyl group” is defined as a branched or unbranched saturated hydrocarbon group of 1 to 24 carbon atoms, such as methyl, ethyl, t-propyl, isopropyl, n-butyl, isobutyl, t-butyl, pentyl, hexyl, heptyl, octyl, decyl, tetradecyl, hexadecyl, eicosyl, tetracosyl and the like.

The term “alkenyl group” is defined as a hydrocarbon group of 2 to 24 carbon atoms and structural formula containing at least one carbon-carbon double bond.

The term “alkynyl group” is defined as a hydrocarbon group of 2 to 24 carbon atoms and a structural formula containing at least one carbon-carbon triple bond.

The term “aryl group” is defined as any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “cycloalkyl group” is defined as a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aralkyl” is defined as an aryl group having an alkyl, alkynyl, or alkenyl group as defined above attached to the aromatic group. An example of an aralkyl group is a benzyl group.

The term “hydroxyalkyl group” is defined as an alkyl, alkenyl, alkynyl, aryl, aralkyl, cycloalkyl, halogenated alkyl, or heterocycloalkyl group described above that has at least one hydrogen atom substituted with a hydroxyl group.

The term “alkoxyalkyl group” is defined as an alkyl, alkenyl, alkynyl, aryl, aralkyl, cycloalkyl, halogenated alkyl, or heterocycloalkyl group described above that has at least one hydrogen atom substituted with an alkoxy group described above.

The term “ester” is represented by the formula —OC(O)R, where R can be an alkyl, alkenyl, alkynyl, aryl, aralkyl, cycloalkyl, halogenated alkyl, or heterocycloalkyl group described above.

The term “carbonate group” is represented by the formula —OC(O)OR, where R can be hydrogen, an alkyl, alkenyl, alkynyl, aryl, aralkyl, cycloalkyl, halogenated alkyl, or heterocycloalkyl group described above.

The term “carboxylic acid” is represented by the formula —C(O)OH.

The term “aldehyde” is represented by the formula —C(O)H.

The term “keto group” is represented by the formula —C(O)R, where R is alkyl, alkenyl, alkynyl, aryl, aralkyl, cycloalkyl, halogenated alkyl, or heterocycloalkyl group described above.

The term “nitro” is represented by the formula —NO₂.

The term “cyano” is represented by the formula —CN.

The term “halogen” is refers to F, Cl, Br or I.

The term “thiol” is represented by the formula —SH. The term “thioalkyl” is represented by the formula —SR, where R is alkyl, alkenyl, alkynyl, aryl, aralkyl, cycloalkyl, halogenated alkyl, or heterocycloalkyl group described above.

The term “amido group” is represented by the formula —C(O)NR₂, where each R is, independently, hydrogen, alkyl, alkenyl, alkynyl, aryl, aralkyl, cycloalkyl, halogenated alkyl, or heterocycloalkyl group described above.

The term “amino group” is represented by the formula —NH₂. The term “alkylamino group” is represented by the formula —NHR or —NR₂, where each R is, independently, alkyl, alkenyl, alkynyl, aryl, aralkyl, cycloalkyl, halogenated alkyl, or heterocycloalkyl group described above.

The term “carbonyl group” is represented by the formula C═O.

The term “ether group” is represented by the formula —R(O)R′, where R and R′ can be, independently, an alkyl, alkenyl, alkynyl, aryl, aralkyl, cycloalkyl, halogenated alkyl, or heterocycloalkyl group described above.

The term “sulfo-oxo group” is represented by the formulas —S(O)₂R, —OS(O)₂R, or, —OS(O)₂OR, where R can be hydrogen, an alkyl, alkenyl, alkynyl, aryl, aralkyl, cycloalkyl, halogenated alkyl, or heterocycloalkyl group described above.

The term “pro-drug” is intended to encompass compounds which, under physiologic conditions, are converted into the therapeutically active agents of the present invention. A common method for making a prodrug is to include selected moieties which are hydrolyzed under physiologic conditions to reveal the desired molecule. In other embodiments, the prodrug is converted by an enzymatic activity of the host animal.

The term “metabolite” refers to active derivatives produced upon introduction of a compound into a biological milieu, such as a patient.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular MPD or MPD analog is disclosed and discussed and a number of modifications that can be made to a number of molecules including the MPD or MPD analog are discussed, specifically contemplated is each and every combination and permutation of MPD or MPD analog and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-B, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### B. COMPOSITIONS AND METHODS

Disclosed are methods and compositions that are related to modulating dopamine content in cells, such as neurons, and modulating the molecules, such as the dopamine receptors D1 and D2, and monoamine transporters, such as DAT and VMAT-2, so that neurons are protected from damage and ultimately death. The disclosed compositions and methods can be used to treat neurodegenerative diseases, such as Parkinson's disease, and they can also be used to delay the onset of neurodegeneration caused by diseases, such as Parkinson's. Neurodegeneration disorders include both idiosyncratic Parkinson's disease and Parkinson's disease caused by known mechanisms; retard neurodegenerative effects of amphetamine-analog administration; retard neurodegenerative effects of pesticide exposure (i.e., botanicals such as rotenone; organochlorines such as kepone; fungisides such as Zineb—which bind VMAT-2 (Vaccari A and Saba P, Eur. J. Pharmacol. 1995; 292:309-314). Neurodegernative disorders can also include hepatochlor. In addition, the disclosed results provide direction as to how and when and what kind of drug to adminster to patients receiving treatment for example, attention deficit disorder. (i.e., this would be important for long-term treatment of attention deficit disorder, as methylphenidate and related molecules would be more desirable than amphetamine analogs).

The compositions and methods can be used to slow the neurodegeneration caused by dopamine accumulation in the cytoplasm, of neurons, and putatitive formation of reactive oxygen species. By reducing the free dopamine concentration in the cytoplasm, for example, dopamine not sequestered by vesicles, the damaging effects of free dopamine can be reduced and in addition, possibly the damage of free dopamine can be reversed.

Also disclosed are systems and methods which can be used to isolate molecules and reagents that are capable of beneficially modulating the free dopamine concentration, by for example, assaying the effect the composition has on the relative position VMAT-2, and the relative activation of dopamine receptors D1 and D2.

The vesicular monoamine transporter-2 (VMAT-2) is the sole transporter responsible for sequestration of intraneuronal monoamines. Amphetamines, presumably including methamphetamine, profoundly affect dopamine storage in synaptic vesicles (Sulzer et al., 1995; Cubells et al., 1994).

DA can cause formation highly reactive neurotoxic reactive species (Graham et al., 1978; Graham, 1978; Maker et al., 1981; Hastings, 1995). The VMAT-2 is a critical regulator of intraneuronal DA content. Neurological damage can be caused by the accumulation of free dopamine in the cytoplasma of cells. This free dopamine can then undergo chemical transformations leading to species, which are highly oxidative and therefore can damage the neuron in which they reside. Numerous investigators have suggested that DA-associated reactive oxygen species formation contribute to the loss of nigrostriatal DA neurons underlying neurodegnerative disorders, such as Parkinson's disease (Cohen, 1990; Fahn and Cohen, 1992; Adams et al., 2001).

Disclosed herein are compositions and methods which are capable of not only reducing the damaging effect of free dopamine. Thus, these compositions and methods can be used to treat neurodegenerative diseases, such as Parkinson's disease.

1. METH Effects on Dopamine Transport in Rats

Vesicular dopamine (DA) uptake can be rapidly altered in synaptic vesicles purified from the striata of stimulant-treated rats. Specifically, a single administration of the plasmalemmal DA transporter inhibitor, cocaine, or the DA D₂ agonist, quinpirole, increases vesicular DA uptake in vesicles purified from the striata of treated rats. These effects of cocaine are prevented by pretreatment with a D₂, but not D₁, DA receptor antagonist. Disclosed are the effects of a mechanistically different psychostimulant, methamphetamine (METH), on vesicular DA uptake. Results demonstrated that a single administration of this DA-releasing agent rapidly and reversibly decreased vesicular DA uptake. The METH-related decrease in vesicular DA uptake was attenuated by pretreatment with the D₂ antagonist, eticlopride, but not the D₁, antagonist, SCH23390. Core body temperature did not contribute to the effects of METH on vesicular DA uptake. Neither quinpirole nor cocaine increased vesicular DA uptake when rats were concurrently treated with METH. These studies provide evidence that psychostimulants rapidly and differentially modify vesicular DA uptake. In addition, these studies demonstrate a complex role for D₂ and DA receptors in altering vesicular DA transport.

Disclosed herein a single METH administration rapidly and reversibly decreased vesicular [³H]DA uptake. The data also suggest a complex role for D₂ receptors regulating VMAT-2 function. These findings provide further evidence that drug treatments that alter DA disposition can rapidly alter vesicular DA uptake, and provide insight into mechanisms underlying the acute physiological regulation of the VMAT-2.

Results presented herein demonstrate a mechanism whereby METH rapidly alters vesicular DA uptake. A single METH injection rapidly (within 1 h) and reversibly (within 24 h) decreased vesicular DA uptake; an effect associated with a decrease Vₘₐₓ and little change in Kₘ for uptake. The METH-induced decrease in uptake was not associated with hyperthermia caused by drug treatment. However, this deficit was mediated, in part, by D₂ receptor activation as evidenced by findings that it was attenuated by pretreatment with the D₂ DA receptor antagonist, eticlopride.

In contrast to the decreases in vesicular DA uptake induced by METH treatment, MPD administration increases vesicular [³H]DA uptake. Like the deficit induced by METH treatment, the MPD-induced increase was prevented by eticlopride pretreatment, suggesting that it, too, is mediated by D₂ receptor activation. Hence, these data imply that depending upon the circumstances, D₂ activation can either increase or decrease vesicular DA uptake.

As noted herein, the effect of quinpirole on vesicular DA uptake did not occur if rats are treated concurrently with METH. Moreover, cocaine did not increase vesicular DA uptake when rats are concurrently treated with METH. Taken together, these data demonstrate that METH mediates its effect on VMAT-2 through mechanisms that were very different from those underlying the effects of cocaine and quinpirole, although each involves D₂ receptor activation. This paradox implies that these drugs either: 1) stimulate different sub-sets of D₂ receptors (i.e., presynaptic or postsynaptic D₂ receptors); and/or 2) activate D₂ receptors in such a manner that the down-stream signaling pathways of these receptors respond differently.

Disclosed herein METH administration rapidly decreases vesicular DA uptake. This rapid and reversible deficit is dependent on D₂ DA receptor activation, and is not associated with METH-induced hyperthermia.

2. MDMA Effects on VMAT-2

Disclosed are the effects of methylenedioxymethamphetamine (MDMA) on the plasmalemmal DA transporter (DAT) and vesicular monoamine transporter-2 (VMAT-2) were assessed. Similar to effects of METH, multiple high-dose MDMA administrations rapidly (within 1 h) decreased plasmalemmal DA uptake, as assessed ex vivo in synaptosomes prepared from treated rats. Unlike effects of multiple METH injections, this deficit was reversed completely 24 h after drug treatment. Also in contrast to effects of multiple METH injections: 1) MDMA caused little or no decrease in binding of the DAT ligand, WIN35428; and 2) neither prevention of hyperthermia nor prior depletion of DA prevented the MDMA-induced reduction in plasmalemmal DA transport. However, a role for phosphorylation is indicated since pretreatment with protein kinase C inhibitors attenuated the deficit caused by MDMA in an in vitro model system. In addition to affecting DAT function, MDMA rapidly decreased vesicular DA transport as assessed in striatal vesicles prepared from treated rats. Unlike effects of multiple METH injections, this decrease partially recovered by 24 h after drug treatment. D2 receptors contributed to this MDMA-induced deficit, whereas hyperthermia did not. Taken together, these results reveal several differences between effects of MDMA and METH on DAT and VMAT-2; differences that may underlie the dissimilar neurotoxic profile of these agents.

Methylenedioxymethamphetamine (DMA; “ecstasy”) has received considerable recent attention due to both its recreational use and neurotoxic potential. Its abuse has increased dramatically over the past several years. For instance, the percentage of 8^{th}-graders reporting having used MDMA in the previous year increased from 1.7% in 1999 to 3.1% in 2000. Among high school seniors, usage increased from 5.6% to 8.2% (Johnston et al., 2000).

Many investigators have shown that high-dose administrations of amphetamine analogs, including MDMA, cause persistent changes in monoaminergic neuronal function, but with varying expressions. For example, multiple injections of methamphetamine (METH) cause dopamine (DA) deficits persisting weeks and months after drug treatment (Koda and Gibb, 1973; Seiden et al., 1976; Hotchkiss et al., 1979; Morgan and Gibb, 1980; Eisch et al., 1992). In contrast, MDMA is far less toxic to DA systems (Johnson et al., 1988; Insel et al., 1989). In addition, we demonstrated recently that multiple high-dose injections of each of these agents also cause a rapid (within 1 h) decrease in plasmalemmal DA transport function (Fleckenstein et al., 1997; Kokoshka et al., 1998; Metzger et al., 1998).

It is well established that high-dose administration of the amphetamine analog, METH, causes persistent DA deficits persisting months and even years after drug treatment in rodents, nonhuman primates, and perhaps humans (Buening and Gibb, 1974; Seiden et al., 1976; Hotchkiss et al., 1979; Morgan and Gibb, 1980; Eish et al., 1992; Wilson et al., 1996; Villemagne, 1998). In contrast, administration of the amphetamine analog, MDMA, is far less toxic to DA systems (Johnson et al., 1988; Insel et al., 1989).

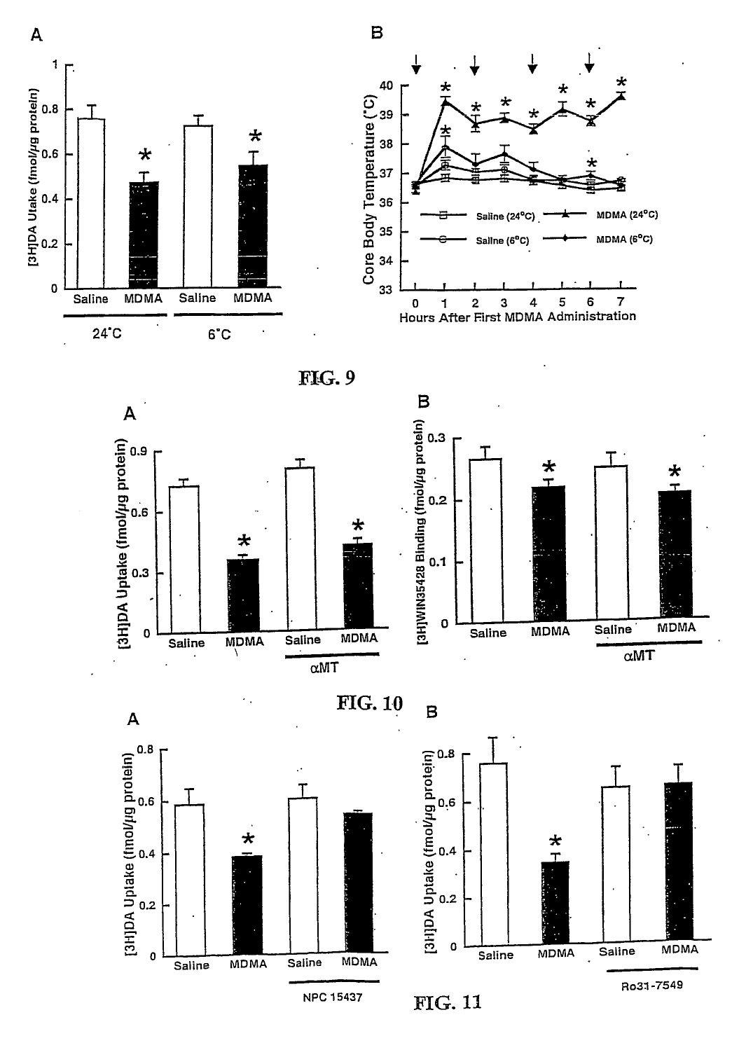

Disclosed herein are differences between the effects of multiple administrations of METH and MDMA (both administered at doses of 10 mg/kg, s.c., 4 injections at 2-h intervals) on plasmalemmal DA uptake. Specifically, the magnitude of the decrease caused by MDMA treatment (35-55%) is less than that observed 1 h after multiple METH administrations (≈70-80%; Kokoshka et al., 1998; Fleckenstein et al., 1999; Metzger et al., 2000). Moreover, the decrease observed 1 h after MDMA treatment recovers completely after 24 h (FIG. 8), whereas the decrease caused by METH only recovers to ≈60% of control values (Kokoshka et al., 1998). In addition, at least one component of the deficit in plasmalemmal DA uptake caused by METH treatment is associated with a decrease in WIN35428 binding (Kokoshka et al., 1998), while multiple MDMA injections had little or no acute effect on the binding of the plasmalemmal DAT ligand. Finally, neither depletion of DA nor prevention of hyperthermia attenuated the acute effects of MDMA on plasmalemmal DA uptake (FIGS. 9 and 10). This is in contrast to METH in that both hyperthermia and DA contribute, in part, to the deficit in plasmalemmal DA uptake caused by multiple administrations of the stimulant (Metzger et al., 2000).

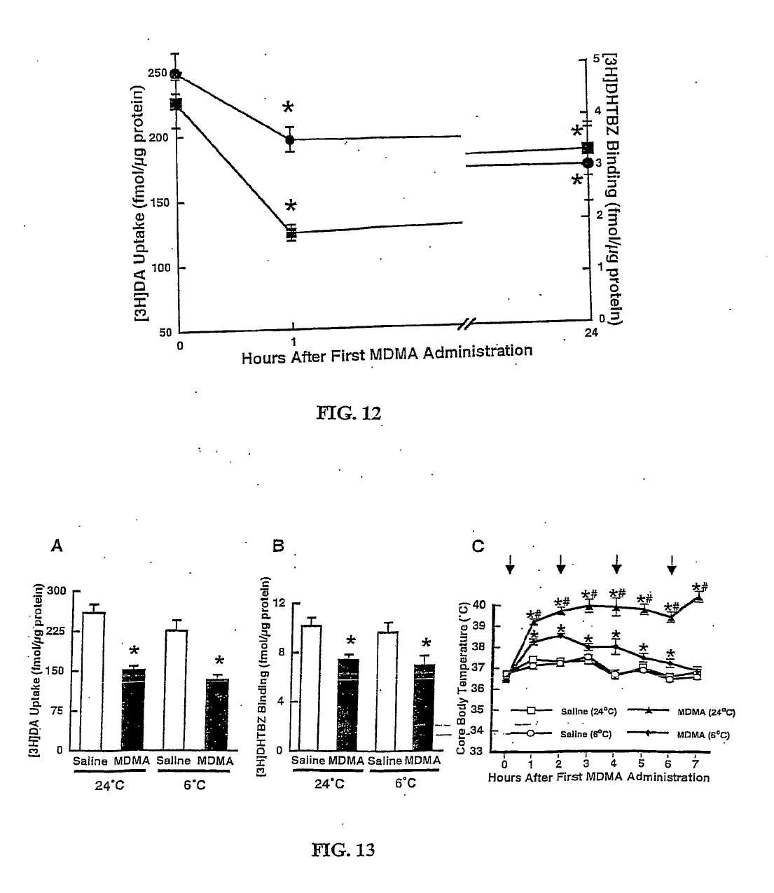

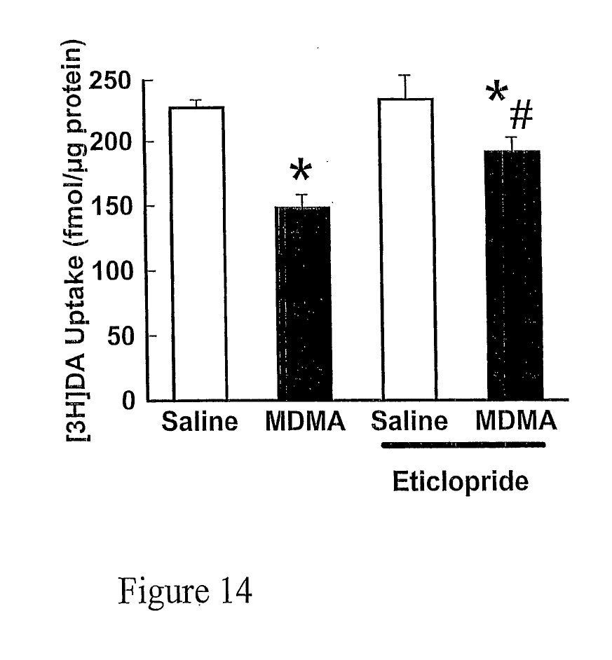

Not only are there significant differences between the effects of METH and MDMA on plasmalemmal DA uptake, but also vesicular DA uptake. For instance, MDMA causes deficits that are lesser in magnitude than those observed after METH treatment (25-30% as shown in FIGS. 12-13 for MDMA vs. ≈65% after METH treatment (Brown et al., 2000)). Moreover, the effect of MDMA was substantially reversed 24 h after treatment, whereas the deficits in vesicular DA uptake caused by multiple injections with METH largely persist 24 h later (Brown et al., 2000). Interestingly, DA contributes to the deficits in vesicular DA uptake caused by multiple MDMA injections (FIG. 14).

Although there were several differences between effects of multiple METH and MDMA administrations, the acute effects of a single METH injection (15 mg/kg) largely resemble the acute effects of multiple MDMA treatments. Specifically, both phenomena: 1) are reversed 24 h after treatment (Fleckenstein et al., 1997; FIGS. 8 and 9) occur independently of DA and of drug-induced hyperthermia (Metzger et al., 2000; FIGS. 9-10). Hence, it might be predicted that like multiple MDMA administrations, a single METH injection would not cause long-term DA deficits. Accordingly, it has been demonstrated that a single 15 mg/kg METH injection does not effect long-term decreases in tyrosine hydoxylase activity; an indicator of the integrity of DA neuronal function (Kogan et al., 1976).

Results presented in FIG. 11 demonstrate that preincubation with NPC15437, as well as another PKC inhibitor (Ro-31-7549), prevents the deficits induced by MDMA application as well. These data indicate that similar mechanisms may underlie the effects of a single METH and multiple MDMA treatments.

Multiple administrations of MDMA and METH differentially alter plasmalemmal and vesicular DA uptake. MDMA and METH differentially alter vesicular DA uptake.

3. METH Effects of Dopamine Transport in Mice

Results reveal that methamphetamine treatment rapidly (within 1 h) decreased mouse vesicular dopamine uptake; a phenomenon associated with a sub cellular redistribution of VMAT-2 immunoreactivity. Both methamphetamine-induced hyperthermia and D₂ dopaminergic receptor activation contributed to the stimulant-induced deficits in vesicular dopamine uptake. Multiple high-dose administrations of methylenedioxymethamphetamine (MDMA) also rapidly decreased vesicular dopamine uptake. In contrast to methamphetamine, this MDMA-induced decrease was reversed 24 h after drug treatment. Noteworthy are findings that in contrast to the releasing agents methamphetamine and MDMA, the dopamine reuptake inhibitors, methylphenidate and cocaine, rapidly (within 1 h) increased vesicular dopamine uptake.

Disclosed herein multiple methamphetamine administrations rapidly similarly decrease dopamine uptake in vesicles purified from mouse striata. As observed in rats, this deficit persists 24 h, and dopamine D₂ receptors contribute to this deficit. The disclosed results also indicate methamphetamine-induced hyperthermia contributed to the rapid decrease in vesicular dopamine uptake caused by multiple administrations of the stimulant to mice.

The findings presented in FIG. 16 indicate that high-dose methamphetamine treatment (4×10 mg/kg, s.c., 2-h intervals) was without effect on total VMAT-2 protein immunoreactivity in whole synaptosomes (i.e., the P2 fraction) prepared from the striata of treated mice. However, when the synaptosomes were lysed and fractionated into non-membrane associated (S3) fractions and membrane-associated (P3), decreases and slight (albeit insignificant) increases, respectively, in VMAT-2 immunoreactivity were observed. Taken together, these data suggest that methamphetamine is effecting a redistribution of VMAT-2 within nerve terminals.

As observed in rats, results presented in FIG. 5 demonstrate that multiple administrations of MDMA (4 injections, 10 mg/kg/injection, s.c.), rapidly decreased mouse vesicular dopamine uptake. Also similar to findings in rats, this decrease was lesser in duration. These findings are of interest in that this same MDMA regimen caused little (i.e., only a 13%) decrease in dopaminergic neuronal function as assessed by measuring tissue dopamine contents 7 d later. In contrast, the multiple high-dose methamphetamine regimen used in the present study causes profound (>50%) dopaminergic damage as assessed days after treatment by measuring dopamine content, dopamine transporter binding and/or tyrosine hydroxylase activity. Taken together, these data indicate that stimulants with acute effects on VMAT-2 that are lesser in magnitude than those of METH are less likely to be neurotoxic.

A single injection of methylphenidate or cocaine increased vesicular dopamine uptake as assessed in vesicles prepared 1 h after treatment.

Disclosed herein the data indicate that drugs that increase vesicular dopamine uptake can be neuroprotective in these model systems. Interestingly, it has been demonstrated that pre- and/or post-treatment with dopamine reuptake inhibitors, including methylphenidate can protect against the long-term dopaminergic deficits caused by methamphetamine treatment (C. J. Schmidt, and J. W. Gibb, Eur. J. Pharmacol. 109 (1985) 73-80).

4Different effects of METH and cocaine on VMAT

High-dose administration of cocaine or methamphetamine to rats acutely (≦24 h) alters vesicular dopamine transport. Disclosed herein there is a differential redistribution of the vesicular monoamine transporter-2 (VMAT-2) within striatal synaptic terminals after drug treatment. In particular, cocaine shifts VMAT-2 protein from a synaptosomal membrane fraction to a vesicle-enriched fraction, as assessed ex vivo in fractions prepared from treated rats. In contrast, methamphetamine treatment redistributes VMAT-2 from a vesicle-enriched fraction to a location that is not retained in a synaptosomal preparation. These data indicate that psychostimulants acutely and differentially affect VMAT-2 subcellular localization.

Cocaine administration causes a redistribution of VMAT-2 protein from the P3 to the S3 fraction. In these experiments, the total amount of VMAT-2 protein in the P3 in untreated animals is ≈70% of that found in the P2 fraction. Hence, the relatively small decrease in P3 VMAT-2 immunoreactivity after cocaine treatment would be expected to result in a large increase in S3 (given that the total amount of protein in the P2 fraction is not altered by cocaine treatment). Previous studies demonstrated that total VMAT-2 levels are not changed by cocaine administration in brain homogenate or slice preparations. The disclosed data are consistent with these previous data since no changes in total synaptosomal VMAT-2 were detected after cocaine treatment. Moreover, the data demonstrate that cocaine can redistribute VMAT-2: a phenomenon that would not be detected when assessing total VMAT-2 protein levels.

In contrast to the effects of cocaine on VMAT-2, results presented in FIG. 22 demonstrate that methamphetamine treatment largely decreased VMAT-2 immunoreactivity in the S3 fraction. This was concurrent with a moderate decrease in P2 VMAT-2 and no change in P3 VMAT-2 levels. This decrease in S3 and P2 VMAT-2 may suggest trafficking from the P2 fraction altogether (i.e. trafficking out of the portion of nerve terminal retained in a synaptosomal preparation) since decreases observed in the P2 and S3 fraction are not likely due to degradation of protein (Hogan et al., 2000; Wilson et al., 1996a). Interestingly, amphetamine increases the phosphorylation of synapsin thereby dissociating vesicles from actin filaments (Iwata et al., 1996, 1997). Competitive inhibition of synapsin (a phenomenon that would presumably mimic synapisn phosphorylation) reduces the number of synaptic vesicles within the nerve terminal (Augustine et al., 1999).

The disclosed results demonstrate that cocaine and methamphetamine differentially affect the subcellular distribution of VMAT-2, and presumably synaptic vesicles. These drugs differentially affect the trafficking of VMAT-2 with respect to the S3 fraction (methamphetamine out of and cocaine into), which suggests that these drugs differentially alter trafficking of vesicles between different cellular locations. The present data demonstrate that VMAT-2 can be differentially redistributed among subcellular fractions.

5. MPD Redistributes VMAT

Methylphenidate (MPD) inhibits dopamine (DA) transporter function. In addition to this effect, disclosed herein MPD increases vesicular [³H]DA uptake and binding of the vesicular monoamine transporter-2 (VMAT-2) ligand, dihydrotetrabenazine (DHTBZ), in a dose- and time-dependent manner in purified striatal vesicles prepared from treated rats. This change did not result from residual MPD introduced by the original in vivo treatment, as application of MPD in vitro (≦1 μM) was without effect, and higher concentrations decreased, vesicular [³H]DA uptake. In addition, MPD treatment increased and decreased VMAT-2 immunoreactivity in striatal vesicle subcellular and plasmalemmal membrane fractions, respectively. The MPD-induced increase in both VMAT-2 immunoreactivity and DHTBZ binding was attenuated by pretreatment in vivo with either the DA D₁ receptor antagonist, SCH23390, or the DA D₂ receptor antagonist, eticlopride. Coadministration of these antagonists in vivo inhibited completely the MPD-induced increase in DHTBZ binding in the purified vesicular preparation. These results indicate a role for DA in the MPD-induced redistribution of VMAT-2.

Methylphenidate (MPD) is one of the most commonly prescribed psychostimulants in the United States. Its primary clinical use is for the treatment of attention deficit hyperactivity disorder (ADHD; Challman and Lipsky, 2000; Zuddas et al., 2000), which is estimated to affect 3-5% of children in the United States (Pincus et al., 1995). There has been an increase in the illicit use of this stimulant presumably due to its pharmacological similarity to other drugs of abuse, such as cocaine. Specifically, MPD inhibits DA transporter function (Ritz et al., 1987; Pan et al., 1994; Izenwasser et al., 1999) and thereby increases extracellular DA levels (Hurd and Ungerstedt, 1989; Butcher et al., 1991).

The vesicular monoamine transporter-2 (VMAT-2) is responsible for the sequestration of cytoplasmic dopamine (Erickson et al., 1992) and is an important regulator of DA neurotransmission. Disclosed herein a single administration of MPD rapidly and reversibly increases vesicular [³H]DA uptake and binding of the VMAT-2 ligand, [³H]dihydrotetrabenazine (DHTBZ) binding. MPD treatment also increases VMAT-2 protein levels in a striatal vesicle subcellular preparation. These MPD-induced increases in vesicular [³H]DA sequestration, [³H]DHTBZ binding and VMAT-2 protein levels are mediated by both DA D₁ and D₂ receptor activation. These phenomena represent a MPD-induced redistribution of vesicles within nerve terminals that is consistent with an alteration intraneuronal DA distribution.

The disclosed data demonstrate that MPD increases vesicular [³H]DA uptake and [³H]DHTBZ binding rapidly and reversibly, as assessed in purified striatal vesicles prepared from treated rats. The MPD-induced effects are attenuated by pretreatment with eticlopride. Pretreatment with SCH23390 attenuated the MPD-induced increases in vesicular [³H]DA uptake and [³H]DHTBZ binding, but it did not prevent the cocaine-induced increases in VMAT-2 activity (Brown et al., 2001). The coadministration of SCH23390 and eticlopride completely inhibited the MPD-induced increases in VMAT-2 fucntion. Hence, unlike the cocaine phenomenon, both DA D₁ and D₂ receptor activation contribute to the increase in vesicular [³H]DA uptake and [³H]DHTBZ binding after MPD treatment.

The data demonstrate that MPD treatment increases and decreases VMAT-2 immunoreactivity in the vesicular subcellular and plasmalemmal membrane fractions, respectively, indicating that MPD redistributes VMAT-2 protein, and synaptic vesicles, between a subcellular pool and the plasma membrane. In accordance with results of the [³H]DHTBZ binding studies, DA D₁ and D₂ receptor activation contribute to the MPD-induced increase in VMAT-2 immunoreactivity in the vesicular subcellular fraction since this increase was prevented by SCH23390 or eticlopride pretreatment (FIGS. 27 and 28).

It has been demonstrated that DA D₂ receptors are negatively coupled to cAMP (Stoof and Kebabian, 1981; Vallar and Meldolesi, 1989), and that a decrease in cAMP leads to a decline in protein kinase A (PKA) activation (Beavo et al., 1974). Synaptic vesicles are tethered to cytoskeleton fibers via synapsin, and synapsin is phosphorylated by protein PKA or calmodulin kinase (Turner et al., 1999). Once synapsin becomes phosphorylated, vesicles traffic from the cytoplasm to the plasma membrane (Turner et al., 1999). Consequently, a DA D₂ receptor-mediated decrease in PKA activation could cause less synapsin to be phosphorylated and thereby increase the amount of synaptic vesicles tethered to cytoskeletal filaments. This increase in tethered vesicles is consistent with the increase in the quantity of purified vesicles disclosed herein.

The data disclosed herein demonstrate that a single administration of MPD rapidly and reversibly increases vesicular [³H]DA uptake and [³H]DHTBZ binding by activating both DA D₁ and D₂ receptors.

6. MPD Reverses METH Neurodegeneration

Disclosed herein MPD post-treatment both prevents the persistent DA deficits and reverses the acute decreases in vesicular DA uptake and VMAT-2 ligand binding caused by METH treatment. In addition, MPD post-treatment reverses the acute decreases in vesicular DA content caused by METH treatment. Taken together, these findings suggest that MPD prevents persistent METH-induced DA deficits by redistributing vesicles and the associated VMAT-2 protein and affecting DA sequestration.

High-dose methamphetamine (METH) administration causes persistent dopamine (DA) deficits in rodents, non-human primates and humans (for review, see Fleckenstein et al., 2000). DA, per se, likely contributes to this damage, as it is attenuated by pretreatment of rats with the DA synthesis inhibitor, α-methyl-p-tyrosine (Gibb and Kogan, 1979; Wagner et al., 1983; Schmidt et al., 1985b). Intraneuronal DA has been suggested to be of particular importance, as METH application causes oxygen radical formation within ventral midbrain culture-containing DA neurons (Cubells et al., 1994).

Intraneuronal DA levels are regulated largely by the vesicular monoamine transporter-2 (VMAT-2), as this carrier transports DA into synaptic vesicles for storage.

As described above, high-dose METH administration causes persistent DA deficits in rodents, non-human primates and humans.

Disclosed herein both pre- and post-treatment with DA reuptake inhibitors attenuate the persistent DA deficits caused by METH treatment. The later finding (i.e., that post-treatment with DA reuptake inhibitors can protect against METH toxicity) is of particular importance, as it indicates the existence of a reversible process occurring in the first few hours after METH treatment that contributes to the long-term DA deficits caused by the stimulant.

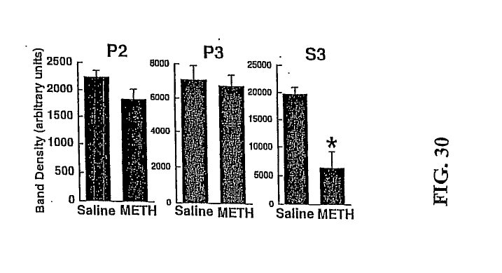

The results presented in FIG. 30 demonstrated that multiple METH injections (4 injections of 7.5 mg/kg/injection, 2-h intervals) rapidly (within 1 h) decrease VMAT-2 protein levels in this preparation. Slight decreases in VMAT-2 immunoreactivity were also observed in the P2 (synaptosomal) fraction from which the vesicles were obtained, with no change in the membrane fraction (P3). A similar phenomenon occurs with higher doses of METH (4 injections of 10 mg/kg/injection, 2-h intervals), except that METH treatment decreased significantly VMAT-2 immunoreactivity in the P2 fraction by 25%.

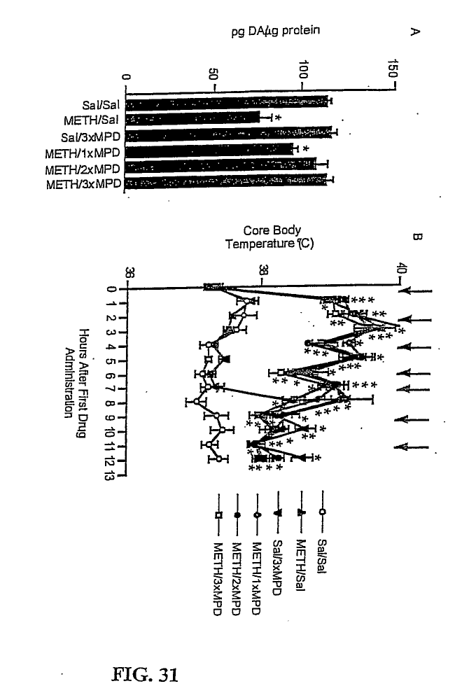

Results presented in FIG. 31 demonstrate that in addition to causing rapid alterations in VMAT-2, METH treatment causes the expected persistent DA deficits. This long-term consequence was inhibited by post-treatment with another DA reuptake inhibitor, MPD. MPD was selected for study as it is an agent with a wide margin of safety that is often used as treatment for attention deficit hyperactivity disorder (for review, see Challman and Lipsky, 2000). Results presented in FIG. 2B demonstrate MPD did not prevent the hyperthermia caused by METH-treatment.

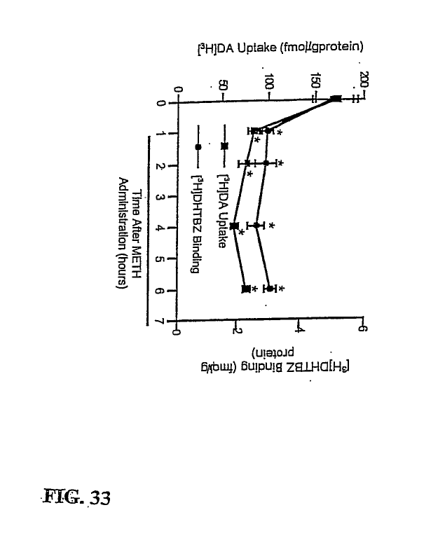

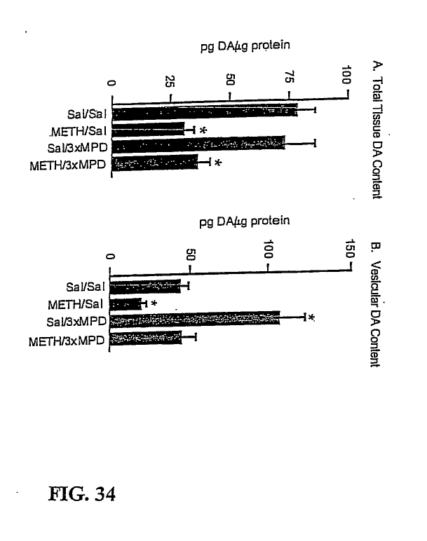

In addition to preventing the persistent DA deficits caused by METH treatment, results presented in FIG. 33 demonstrate that post-treating animals with MPD reversed the acute decreases in vesicular DA uptake and DHTBZ binding that occurs in the first hours after METH treatment. The neuroprotective effect of MPD is consistent with trafficking of VMAT-2 and associated vesicles to a subcellular region left devoid of VMAT-2 activity because of METH treatment. Thus, MPD would increase vesicular DA sequestration in that region and perhaps compensate for any consequent METH-associated accumulation of cytoplasmic DA. This is supported by the finding that MPD increases vesicular DA content as assessed in vesicles prepared from the striata of treated rats without altering total tissue DA concentrations (FIG. 34). This suggests that MPD treatment redistributed DA within the terminals, presumably as a consequence of the redistribution of vesicles. In contrast, METH treatment decreased both tissue and vesicular DA content, likely because of a deficit in vesicular DA sequestration and an inhibition of tyrosine hydroxylase after the multiple METH injection treatment regimen. Importantly, the METH-induced decrease in vesicular DA content was reversed by the same MPD post-treatment regimen that reversed: 1) the acute (1 h) METH-induced decrease in vesicular DA uptake and DHTBZ binding; and 2) the persistent (and likely neurotoxicity-related) DA deficits caused by METH treatment.

7. Dopamine Transport

The DAT is a principal regulator of DA disposition (i.e., of intra- and extra-neuronal DA concentrations), and changes in DA disposition resulting from amphetamine analogs putatively contribute to their ability to cause long-term DA deficits in the striatum. In particular, others (Cubells et al., 1994; Fumagalli et al., 1999; LaVoie et al., 1999) and we (for review, see Fleckenstein et al., 2000) have hypothesized that psychostimulants may redistribute DA from the reducing environment within synaptic vesicles to extravesicular intra-cellular oxidizing environments, thus causing the formation of oxygen radicals and reactive metabolites within DA neurons that trigger selective DA terminal loss. Accordingly, DA-releasing agents that rapidly decrease DAT function (i.e., METH; Fleckenstein et al., 1997; Kokoshka et al., 1998) may interfere with DAT function and attenuate DA effilux, thereby “trapping” DA in intraneuronal spaces where it can damage DA nerve terminals. Hence, an understanding of the effect of psychostimulants on DAT is important.

In addition to the DAT, the VMAT-2 is a significant regulator of intraneuronal DA concentrations. Presumably, a decrease in the function of the VMAT-2 impedes the sequestration of DA into synaptic vesicles, and may, therefore, increase cytoplasmic DA concentrations. Accordingly, a stimulant-induced decrease in vesicular uptake would presumably contribute to effects leading to persistent DA neuronal deficits. Consistent with this hypothesis, increased METH neurotoxicity in heterozygous VMAT-2 knock-out mice has been reported (Fumagalli et al., 1999). Hence, like DAT, an understanding of the effects of stimulants on VMAT-2 is importantCompositions

8. Compositions Reducing Neurodegeneration

Disclosed herein are compositions which are capable of reducing neurodegeneration, as well as aiding in the protection from neurodegeneration. These compositions can therefore be used to treat neurodegenerative diseases, such as Parkinson's disease.

In certain embodiments, the compositions alter the activity of VMAT-2, increasing the VMAT-2 activity. In certain embodiments the compositions alter the distribution of VMAT-2 containing vesicles within a cell, such as a neuron, and by this redistribution are able to reduce neurodegeneration. In certain embodiments the disclosed compositions are compositions that are able to effect the VMAT-2 distribution in conjunction with activation of the dopamine receptors D1 and/or D2. In certain embodiments the compositions can be dopamine transporter (DAT) reuptake inhibitors. It is also understood that in certain embodiments the compositions can be D1 and/or D2 agonists.

Disclosed are compositions, wherein the compositions shift the VMAT-2 protein from a synaptosomal membrane fraction to a vesicle-enriched fraction. This can be assessed ex vivo in fractions prepared from treated rats. Also disclosed are compositions wherein both the DA D₁ and D₂ receptor activation contribute to the increase in vesicular [³H]DA uptake and [³H]DHTBZ, such as MPD.

9. Compositions Related to MPD

Any of the compounds represented by formula I can be used in any of the methods described herein, 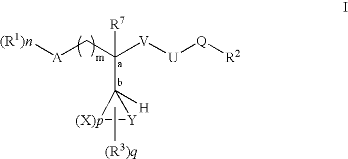
wherein

A represents a cycloalkyl group, heterocycloalkyl group, an aryl group or heteroaryl group;

U is absent or when U is present, U represents —C(═O)—, —C(═S)—, —P(═O)(OR⁵)—, —S(O₂)— or —S(O)—;

V is absent or when V is present, V is NR⁶, O or S;

Q is absent or when Q is present, V is NR⁶, O or S;

Y represents NR⁴, O or S;

X is, independently, C, N, S, Se or O;

R¹ is, independently, hydrogen, aryl, alkyl, alkoxy, hydroxy, hydroxyalkyl, aralkyl, halogen, cyano, aldehyde, ketone, ester, carbonate, amido, amino, alkylamino, nitro, thiol, thioalkyl or a sulfo-oxo group;

R² is hydrogen, aryl, alkyl, aralkyl, alkoxy, hydroxy, hydroxyalkyl, halogen, ester, carbonate, amido, amino, alkylamino, thiol or thioalkyl;

R³ is, independently, hydrogen, aryl, alkyl, aralkyl, alkoxy, hydroxy, hydroxyalkyl, halogen, cyano, aldehyde, ketone, ester, carbonate, amido, amino, alkylamino, nitro or a sulfo-oxo group;

wherein the ring formed by X, Y and carbon b optionally contains a carbon-carbon double or carbon-oxygen double bond;

R⁴ is hydrogen, alkyl, keto, aryl, aralkyl, heteroaryl or heteroaralkyl;

R⁵, R⁶ and R⁷ are, independently, hydrogen, alkyl, alkenyl, aryl, heteroaryl, aralkyl or heteroaralkyl;

m is an integer of from 0 or 1;

n is an integer of from 0 to 7;

p is an integer of from 3 to 6;

q is an integer of from 0 to 16;

the stereochemistry at carbon a and carbon b is R or S;

or a pharmaceutically acceptable salt, pro-drug or metabolite thereof.

Examples of compounds having the formula I are represented by formulae II and III

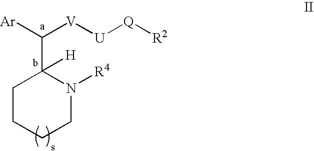

wherein

U is absent or when U is present, U represents —C(═O)—, —C(═S)—, —P(═O)(OR⁵)—,

—S(O₂)— or —S(O)—;

V is absent or when V is present, V is NR⁶, O or S;

Q is absent or when Q is present, V is NR⁶, O or S;

R² is hydrogen, aryl, alkyl, aralkyl, alkoxy, hydroxy, hydroxyalkyl, halogen, ester, carbonate, amido, amino, alkylamino, thiol or thioalkyl;

R⁵ and R⁶ are, independently, hydrogen, alkyl, alkenyl, aryl, heteroaryl, aralkyl or heteroaralkyl;

s is an integer of from 0 to 2;

Ar is a substituted or unsubsituted aryl or heteroaryl group;

the stereochemistry at carbon a and carbon b is R or S;

or a pharmaceutically acceptable salt, pro-drug or metabolite thereof; and

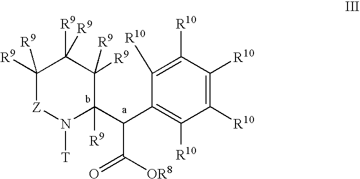

wherein

R⁸ is hydrogen, aryl, alkyl, alkenyl, hydroxyalkyl, aralkyl, aldehyde, ketone, cycloalkyl, heteroaryl or the pharmaceutically acceptable salt thereof;

R⁹ and R¹⁰ are, independently, hydrogen, aryl, alkyl, alkenyl, alkoxy, hydroxy, hydroxyalkyl, aralkyl, halogen, cyano, aldehyde, ketone, ester, carbonate, amido, amino, alkylamino, thiol, thioalkyl, nitro or a sulfo-oxo group; Z is —CH₂— or —C(═O)—; T is hydrogen or —C(═O)—N(R¹¹)₂, wherein R¹¹ is, independently, hydrogen, aryl, alkyl or aralkyl;

and the stereochemistry at carbon a and carbon b is R or S,

or a pharmaceutically acceptable salt, pro-drug or metabolite thereof.

The stereochemistry at carbons a and b of formulae I-III can vary depending upon the methodology used to prepare and isolate the compounds. There are numerous methods for interconverting the diastereomers of the compounds and for resolving the enantiomers of compounds having the formulae I-III. Representative methods have been described in U.S. Pat. No. 2,507,631 to Hartmann; U.S. Pat. No. 2,838,519 to Rometsch; U.S. Pat. No. 2,957,880 to Rometsch; British Patent Nos. 788,226 and 878,167; Soviet Patent No. 466,229 to Yakhontov et al.; International Publication No. WO9735836 of Fox et al.; International Publication No. WO9728124 to Langston et al.; Panizzon, 1944, Helv. Chim. Acta 27: 1748-1756; Naito et al., 1964, Chem. Pharm. Bull. 12: 588-590; Deutsch et al., 1996, J. Med. Chem. 39: 1201-1209; Earle et al., 1969, J. Chem. Soc. (C) 2093-2098); International Publication No. WO0825902 to Faulconbridge et al.; Patrick et al., 1987, J. Pharmacol. Exp. Therapeut. 241: 152-158; International Publication No. WO9727176 of Harris et al.; International Publication No. WO9825902 to Zavareh. The contents of these publications are incorporated herein by reference in their entireties.

In the case of formulae I-III, there are four possible diastereoisomers. For example, the stereochemistry at carbon a can be R and the stereochemistry at carbon b can be substantially R or substantially S. Alternatively, the stereochemistry at carbon a can be S and the stereochemistry at carbon b can be substantially R or substantially S. The term “substantially” refers to an enantiomeric excess (ee) greater than 50% at either carbon a or b. For example, the ee at carbon a or b of formulae I-III can be greater than 50%, 60%, 70%, 80%, 90%, 95%, 99% or 99.5%. It is also possible to have a mixture of two or more diastereoisomers. As an illustration of the four possible diastereoisomers represented by formulae I-III, Scheme 1 provides the structure of each diastereoisomer of methyl phenidate.

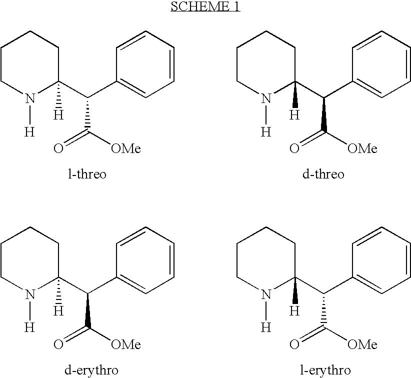

Alternatively, a racemic mixture of a compound having the formulae I-III can be used in the methods described herein.

Certain embodiments of compounds of formulae I-III may contain a basic functional group, such as amino or alkylamino, and thus, can be utilized in a free base form or as pharmaceutically acceptable salt forms derived from pharmaceutically acceptable organic and inorganic acids.

The pharmaceutically acceptable salts of the subject compounds I-III include the conventional nontoxic salts and/or quaternary ammonium salts of the compounds, e.g., from non-toxic organic or inorganic acids. For example, such conventional nontoxic salts include those derived from inorganic acids including, but not limited to, hydrochloride, hydrobromic, sulfuric, sulfamic, phosphoric, nitric, and the like. In another embodiment, the salts of formulae I-III can prepared from organic acids including, but not limited to, acetic, 2-acetoxybenzoic, ascorbic, benzene sulfonic, benzoic, chloroacetic, citric, ethane disulfonic, ethane sulfonic, formic, fumaric, gluconic, glutamic, glycolic, hydroxymaleic, isothionic, lactic, maleic, malic, methanesulfonic, oxalic, palmitic, phenylacetic, propionic, salicyclic, stearic, succinic, sulfanilic, tartaric, toluenesulfonic, and the like.

In another embodiment, when the compound of formulae I-III contains a basic nitrogen-containing group, the basic nitrogen-containing group can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Any of the compounds and methods of treatment disclosed in U.S. Pat. No. 2,507,631 to Hartmann; U.S. Pat. No. 2,838,519 to Rometsch; U.S. Pat. No. 2,957,880 to Rometsch; British Patent Nos. 788,226 and 878,167; Soviet Patent No. 466,229 to Yakhontov et al.; International Publication No. WO9735836 to Fox et al.; International Publication No. WO9728124 to Langston et al.; Panizzon, 1944, Helv. Chim. Acta 27: 1748-1756; Naito et al., 1964, Chem. Pharm. Bull. 12: 588-590; Deutsch et al., 1996, J. Med. Chem. 39: 1201-1209; Earle et al., 1969, J. Chem. Soc. (C) 2093-2098); International Publication No. WO9825902 to Faulconbridge et al.; Patrick et al., 1987, J. Pharmacol. Exp. Therapeut. 241: 152-158; International Publication No. WO9727176 to Harris et al.; International Publication No. WO9825902 to Zavareh; International Publication No. WO0217919 of Wiig et al.; International Publication No. WO9936403 of Winklter et al.; International Publication No. WO0217920 of Wiig et al.; U.S. Pat. No. 5,859,249 to Seido et al.; U.S. Pat. No. 6,008,358 to Nishikawa et al.; U.S. Pat. No. 5,733,756 to Zeitlin et al.; U.S. Pat. No. 6,025,502 to Winklter et al.; U.S. Pat. No. 6,255,325 to Dariani et al., U.S. Patent Application Publication No. US 2003/0073681 A1, U.S. Patent Application Publication No. US 2003/0050309 A1, and U.S. Patent Application Publication No. US 2002/0042357 A1 can be used in the methods described herein. The contents of these publications are incorporated herein by reference in their entireties.

In the case of formula III, in one embodiment, each R⁹ is hydrogen and each R¹⁰ is hydrogen. Alternatively, Z is CH₂ and T is hydrogen in formula III. In another embodiment of formula III, R⁸ is hydrogen or C₁-C₈ alkyl, preferably methyl. In a further embodiment of formula III, the stereochemistry at carbons a and b is R. In another embodiment of formula III, R⁸ is C₁-C₈ alkyl, each R⁹ is hydrogen, each R¹⁰ is hydrogen, Z is CH₂, T is hydrogen, and the stereochemistry at carbons a and b is R.

Examples of compounds useful in the methods described herein include, but are not limited to, phenylpiperidin-2-yl-acetic acid; (4-hydroxy-phenyl)-(piperidin-2-yl)-acetic acid methyl ester; (4-hydroxy-phenyl)-(piperidin-2-yl)-acetic acid; (6-oxo-piperidin-2-yl)-phenyl-acetic acid methyl ester; (6-oxo-piperidin-2-yl)-phenyl-acetic acid, (4-hydroxy-phenyl)-(6-oxo-piperidin-2-yl)-acetic acid methyl ester; 2-[2-(4-hydroxy-phenyl)-2-(6-oxo-piperidin-2-yl)-acetylamino]-ethanesulfonic acid; (5-hydroxy-6-oxo-piperidin-2-yl)phenyl-acetic acid; (1-carboamyl-piperidin-2-yl)-phenyl-acetic acid methyl ester; 1-(carboamoyl-piperidin-2-yl)-phenyl-acetic acid; (5-hydroxy-6-oxo-piperidin-2-yl)phenyl-acetic acid methyl ester; (4-hydroxy-6-oxo-piperidin-2-yl)-phenyl-acetic acid methyl ester; 3,4,5-trihydroxy-6-[2-(methoxycarbonyl-phenyl-methyl)-6-oxo-piperidin-4-yloxy]-tetrahydropyran-2-carboxylic acid; 3,4,5-trihydroxy-6-{4-[methoxycarbonyl(6-oxo-piperidin-2-yl)-methyl]-phenoxy}-tetrahydropyran-2-carboxylic acid, 6-[4-(carboxy-piperidin-2-yl-methyl)-phenoxy]-3,4,5-trihydroxy-tetrahydro-pyran-2carboxylic acid; 3,4,5-trihydroxy-6-[6-(methoxycarbonyl-phenyl-methyl)-2-oxopiperidin-3-yloxy]-tetrahydropyran-2-carboxylic acid; 3,4,5-trihydroxy-6-[2-(6-oxopiperidin-2-yl)-2-phenyl-acetoxy]-tetrahydro-pyran-2-carboxylic acid and phenylpiperidin-2-yl-acetic acid ethyl ester.

Also disclosed in certain embodiments are dopamine reuptake inhibitors. Also included are WIN35428 analogs, GBR12909, nomifensine, mazindol, and analogs.

In one embodiment, the compound is l-threo methylphenidate, d-threo methylphenidate, l-erythro methylphenidate or d-erythro methylphenidate, preferably d-threo methylphenidate, which is also referred to as Ritalin®. In another aspect, a compound having the formula III is not used to treate Parkinson's disease.

Any of the methods disclosed in U.S. Pat. No. 2,507,631 to Hartmann; U.S. Pat. No. 2,838,519 to Rometsch; U.S. Pat. No. 2,957,880 to Rometsch; British Patent Nos. 788,226 and 878,167; Soviet Patent No. 466,229 to Yakhontov et al.; International Publication No. WO9735836 to Fox et al.; International Publication No. WO9728124 to Langston et al.; Panizzon, 1944, Helv. Chim. Acta 27: 1748-1756; Naito et al., 1964, Chem. Pharm. Bull. 12: 588-590; Deutsch et al., 1996, J. Med. Chem. 39: 1201-1209; Earle et al., 1969, J. Chem. Soc. (C) 2093-2098); International Publication No. WO9825902 to Faulconbridge et al.; Patrick et al., 1987, J. Pharmacol. Exp. Therapeut. 241: 152-158; International Publication No. WO9727176 to Harris et al.; International Publication No. WO9825902 to Zavareh; International Publication No. WO0217919 of Wiig et al.; International Publication No. WO9936403 of Winklter et al.; International Publication No. WO0217920 of Wiig et al.; U.S. Pat. No. 5,859,249 to Seido et al.; U.S. Pat. No. 6,008,358 to Nishikawa et al.; U.S. Pat. No. 5,733,756 to Zeitlin et al.; U.S. Pat. No. 6,025,502 to Winklter et al.; and U.S. Pat. No. 6,255,325 to Dariani et al. can be used to produce the compounds having the formulae I-III. The contents of these publications are incorporated herein by reference in their entireties.

Any of the compounds represented by formula IV can be used in any of the methods described herein

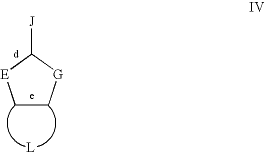

wherein

when d is a single bond, E is S, O, C(R¹¹)₂, or NR¹¹, and when d is double bond, E is CR¹¹ or N;

G is S, O, C(R¹¹)₂, or NR¹¹;

J is hydrogen, C(R¹²)₃, SR¹², OR¹², or N(R¹²)₂;

wherein R¹¹ and R¹² are, independently, hydrogen, aryl, alkyl, aralkyl, alkoxy, hydroxy, hydroxyalkyl, halogen, ester, carbonate, amido, amino, alkylamino, thiol or thioalkyl;

L is a fused substituted or unsubstituted cycloalkyl group, heterocycloalkyl group, aryl group, or heteroaryl group;

d is a single bond or a double bond; and

e is a single bond or a double bond.

In one aspect, the compound has the formula IV, wherein d is a double bond and E is N. In another aspect, G is S. In a further aspect, L in formula IV is a cycloalkyl group such as a cyclobuty group, a cyclopently group, a cyclohexyl group, a cyclohexyl group, or a cyclooctyl group. In another aspect, when L is a cycloalkyl group, the cycloalkyl group can be substituted or unsubstituted. Any of the groups described herein can be attached the cycloalkyl group in this aspect. In one aspect, the cycloalkyl group is substituted with a mono- or disubstituted amino group or unsubstituted amino group, where the amino groups can be substituted with any of the groups defined above. For example, the amino group can be substituted with one or more alkly groups defined herein includeing, but not limited to, methyl, ethyl, propyl, butyl, or pentyl. In one aspect, the amino group is NHPr.

In another aspect, J in formula IV is N(R¹²)₂. In another aspect, each R¹² in formula IV is hydrogen. In a further aspect, d and e in formula IV are double bonds.

In another aspect, compounds having the formula V can be used in any of the methods described herein 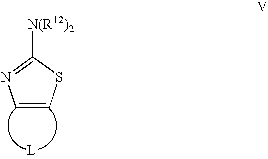
wherein R¹² and L are defined as above. In a further aspect, any of the compounds having the formula VI can be used in any of the methods dexcribed herein 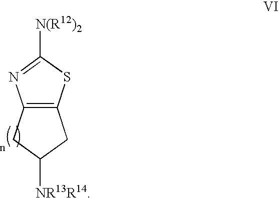
wherein R¹³ and R¹⁴ are hydrogen, aryl, alkyl, aralkyl, hydroxyalkyl, or R¹³ and R¹⁴ form a cycloalkyl group or heterocycloalkyl group, and

n is from 0 to 3.

In one aspect, when the compound is formula VI, n is not 2. In another aspect, the compound Primapraxal (formula VI, each R¹² is hydrogen, R¹³ is hydrogren, R¹⁴ is propyl, and n is 1) is not used in the methods to treat Parkinson's disease.

In another aspect, compounds having the formula VII can be used in any of the methods described herein 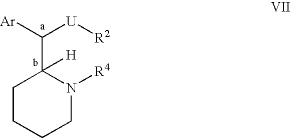
wherein

U is —C(═O)—, —S(O₂)— or —S(O)—;

R² is hydrogen, aryl, aralkyl;

R⁴ is hydrogen, alkyl, keto, aryl, aralkyl, heteroaryl or heteroaralkyl; and the stereochemistry at carbon a and carbon b is R or S.

In another aspect, compounds having the formula VIII can be used in any of the methods described herein 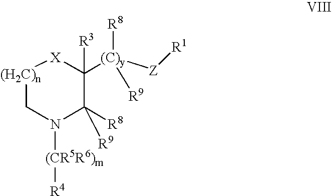
wherein

X represents C(R³)₂, O, S, SO, SO₂, NR², NC(O)R⁷, NC(O)OR², NS(O)₂R⁷, or C═O;

Z represents C(R³)₂, C(O), O, NR, NC(O)OR, S, SO, or SO₂;

m is 1, 2, 3, 4 or 5;

n is 1 or 2;

p is 0, 1, 2, or 3;

y is 0, 1, or 2;

R¹ represents H, alkyl, cycloalkyl, aryl, heteroaryl, aralkyl, or heteroaralkyl;

R¹ represents H, alkyl, aryl, heteroaryl, aralkyl, or heteroaralkyl;

R and R¹ may be connected through a covalent bond;

R² represents independently for each occurrence H, alkyl, fluoroalkyl, aryl, heteroaryl, or cycloalkyl;

R³ represents independently for each occurrence H, alkyl, aryl, OR², OC(O)R², CH₂OR², or CO₂R²; wherein any two instances of R³ may be connected by a covalent tether whose backbone consists of 1, 2, 3, or 4 carbon atoms;

R⁴ represents independently for each occurrence H, alkyl, cycloalkyl, aryl, heteroaryl, alkenyl, or OR;

R⁵ and R⁶ are selected independently for each occurrence from the group consisting of H, alkyl, (CH₂)ₚY, aryl, heteroaryl, F, OR², and OC(O)R²; or an instance of CR⁵R⁶ taken together is C(O);

R⁷ represents alkyl, cycloalkyl, aryl, heteroaryl, aralkyl, or heteroaralkyl;

R⁸ and R⁹ are selected independently for each occurrence from the group consisting of H, alkyl, (CH₂)ₚY, aryl, heteroaryl, F, OR², and OC(O)R²; or an instance of CR⁸R⁹ taken together is C(O);

Y represents independently for each occurrence OR², N(R²)₂, SR₂, S(O)R², S(O)₂R², or P(O)(OR²)₂; any two instances of R² may be connected through a covalent bond;

a covalent bond may connect R⁴ and an instance of R⁵ or R⁶; any two instances of R⁵ and R⁶ may be connected through a covalent bond;

any two geminal or vicinal instances of R⁸ and R⁹ may be connected through a covalent bond;

and the stereochemical configuration at any stereocenter of a compound represented by A is R, S, or a mixture of these configurations.

In another aspect, compounds having the formula IX can be used in any of the methods described herein 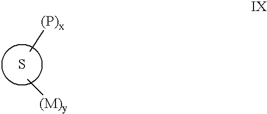
wherein S comprises a scaffold unit; P comprises a pharmacophore unit, wherein x is greater than or equal to two; and M comprises a modifier unit, wherein y is greater than or equal to 0, whereby each one of P and M, for each occurrence, is appended to said scaffold unit, and whereby the polypharmacophore interacts with at least two biological targets. Any of the scaffold units, pharamacore units, and modifier units disclosed in U.S. Patent Application Publication No. 2002/0042357, which is incorporated by reference, can be used in this embodiment.

In one aspect, any of the compounds having the formula I-IX can be used to treat Parkinson's disease.

The compounds having the formulae I-IX are amenable to combinatorial chemistry and other parallel synthesis schemes (see, for example, PCT WO 94/08051, which is incorporated by reference in its entirety). The result is that large libraries of related compounds can be screened rapidly in high throughput assays in order to identify compounds useful in the methods described herein.

A combinatorial library is a mixture of chemically related compounds that can be screened together for a desired property. The preparation of many related compounds in a single reaction greatly reduces and simplifies the number of screening processes that need to be carried out. Screening for the appropriate physical properties can be done by conventional methods. Diversity in the library can be created at a variety of different levels. A variety of techniques are available in the art for generating combinatorial libraries of small organic molecules that fall under formulae I-III, which include Blondelle et al. (1995) Trends Anal. Chem. 14: 83; U.S. Pat. No. 5,359,115; U.S. Pat. No. 5,362,899: U.S. Pat. No. 5,288,514: International Publication No. WO 94/08051; U.S. Pat. No. 5,736,412; U.S. Pat. No. 5,712,171; Chen et al. (1994) JACS 116: 2661; Kerr et al. (1993) JACS 115: 252; and International Publication Nos. WO92/10092, WO93/09668, WO91/07087 and WO93/20242). These publications are incorporated by reference in their entireties. Many variations of the methods disclosed in these publications permit the synthesis of widely diverse libraries having the formulae I-IX. In one embodiment, a library of methylphenidate analogs can be synthesized utilizing a scheme adapted to the techniques described in International Publication No. WO 94/08051, which is incorporated by reference in its entirety.

10. Nucleotides and Related Molecules

There are a variety of molecules disclosed herein that are nucleic acid based, including for example the nucleic acids that encode, for example VMAT-2 and DAT, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, Ni, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

c) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

There are a variety of sequences related to for example, the VMAT-2, DAT, D1 or D2 genes, found in sequence data bases, such as Genbank. These sequences and others are herein incorporated by reference in their entireties as well as for individual subsequences contained therein.

Those of skill in the art understand how to resolve sequence discrepancies and differences and to adjust the compositions and methods relating to a particular sequence to other related sequences (i.e. sequences of VMAT-2, DAT, or is D1 or D2). Primers and/or probes can be designed for any VMAT-2, DAT, or D1 or D2sequence given the information disclosed herein and known in the art. It is understood that homologies and identies can be determined using the sequences disclosed herein.

d) Primers and Probes

Disclosed are compositions including primers and probes, which are capable of interacting with, for example, the VMAT-2, DAT, or D1 or D2 nucleic acids, such as mRNA, as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with, for example, the VMAT-2, DAT, D1 or D2 nucleic acid, such as mRNA, or region of the VMAT-2, DAT, D1 or D2 nucleic acids or they hybridize with the complement of the VMAT-2, DAT, D1 or D2 nucleic acids or complement of a region of the VMAT-2, DAT, D1 or D2 nucleic acids.

e) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(1) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(2) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) orhygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

11. Peptides

a) Protein Variants

As discussed herein there are numerous variants of the VMAT-2, DAT, D1, and D2 proteins that are known and herein contemplated. In addition, to the known functional strain variants there are allelic and functional derivatives of the VMAT-2, DAT, D1, and D2 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative Substitutions,
others are known in the art.

| | |
|---|---|
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:1 sets forth a particular sequence of VMAT-2. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

12. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include reagents, such as cells and a reuptake inhibitor, discussed in certain embodiments of the methods, as well as the buffers and enzymes required to perform assays, such as screeing assays.

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Methods for Isolating Molecules Affecting Free Dopamine Concentration

Disclosed are compositions and methods, which can be used to isolate and identify molecules that are capable of altering the free dopamine concentration in cells, and thus, can be used as reagents for treating Parkinson's disease. It is disclosed herein that a key aspect in reducing the free dopamine concentration so that damaging effects are reduced is the relative positioning and distribution of vesicles capable of sequestering free dopamine. In certain embodiments this positioning is shown herein to be linked to activation of the plasmalemmal dopamine receptors, D1 and D2. Thus, molecules that act as agonists of the D1 and D2 receptors and which alter the positioning of the VMAT-2 containing vesicles are molecules which reduce the damaging effects of free dopamine in a cell. Therefore, disclosed are systems in which VMAT-2 proteins are expressed, along with D1 and D2 dopamine receptors, and which can be then assayed for VMAT-2 positioning and function. It is understood that these methods can be used with a variety of combinatorial chemistry techniques to isolate and identify molecules having the desired function from pools of molecules. The disclosed methods can use the disclosed compositions as controls. For example, the effect MPD has can be used as a standard and molecules being tested or screened can be compared to the MPD effect, either directly or indirectly by referring to the data herein. Cells which express one or more of the compenents can be used as discussed herein to isolate and identify compositions that affect vesicular distribution, VMAT-2 activity, and/or neurodegneration. In addition, the effect of the compositions can be visualized using electron microscopy.

The demonstration that psychostimulants can rapidly and bi-directionally alter VMAT-2 in a mouse as well as a rat provides an additional model for investigating the role of VMAT-2 in effecting stimulant-induced neurotoxicity. Such studies may provide insight not only into the neurotoxicity afforded by methamphetamine, but also deficits resulting from other disorders involving perhaps involving abnormal intraneuronal dopamine distribution such as Parkinson's disease.

It is understood that the disclosed compositions can be made by a variety of methods, and the disclosed herein are compositions produced by those methods having the properties disclosed herein.

2. Compositions Identified by Screening with Disclosed Compositions and Relationships/Combinatorial Chemistry

a) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed herein or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, cells expressing VMAT-2 and D1 and D2 or MPD, are also disclosed. Thus, the products produced using the combinatorial or screening approaches that involve the disclosed compositions, such as, cells expressing VMAT-2 and D1 and D2 or MPD, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerfill molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a portion of D1 or D2 is attached to a DNA binding domain of a transcriptional activation protein, such as Gal 4. By performing the Two-hybrid technique on this type of system, molecules that bind the portion of D1 or D2 can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in interative processes.

b) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, cells expressing VMAT-2 and D1 and D2, or MPD, are also disclosed. Thus, the products produced using the molecular modeling approaches that involve the disclosed compositions, such as, cells expressing VMAT-2 and D1 and D2, or MPD are also considered herein disclosed.

The disclosed compostions and mechanisms can be used in methods of identification of compounds that have the properties of the disclosed compositions. For example, the disclosed compositions and mechanisms and molecular interactions can be used in methods wherein there is a step of incubation with the disclosed compositions and another compound or set of compounds or the compositions can be incubated together. The methods can further comprise a step of assaying for one or more of the activities or characteristics disclosed herein. The methods can also comprise a step of comparison between controls, such as the compositions disclosed herein, a step of identification, a step of synthesis, a step of mansufacture of the compounds, or a addtional steps related to the assays disclsoed herein, for example.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### D. METHODS OF USING THE COMPOSITIONS

1. Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer 58:700-703, (1988); Senter, et al., Bioconjugate Chem. 4:3-9, (1993); Battelli, et al., Cancer Immunol. Immunother. 35:421-425, (1992); Pietersz and McKenzie, Immunolog. Reviews, 129:57-80, (1992); and Roffler, et al., Biochem. Pharmacol 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as MPD, for treating, inhibiting, or preventing Parkinson's for example, the efficacy of the therapeutic MPD can be assessed in various ways well known to the skilled practitioner. For instance, one of ordinary skill in the art will understand that a composition, such as MPD, disclosed herein is efficacious in treating or inhibiting Parkinson's, for example, in a subject by observing that the composition reduces the symptoms of Parkinson's disease.

The compositions that cause a redistribution of VMAT-2 containing vesicles, or inhibit neurodegeneration, disclosed herein may be administered prophylactically to patients or subjects who are at risk for neurodegenerative disorders, such as Parkinson's or drug induced degeneration.

Other molecules that cause a redistribution of VMAT-2 containing vesicles, for example, which do not have a specific pharmacuetical function, but may be used for tracking changes within neurons and may be used as tools to study the function of nerve cells.

The disclosed compositions and methods can also be used for example as tools to isolate and test new drug candidates for a variety of neurodegenerative disorders.

2. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hermatol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

3. Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed molecules, such as MPD and analogs or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Feigner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother. 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

4. Treatment of Other Diseases

Any of the compounds described herein can be used to treat or prevent numerous diseases other than Parkinson's. In one aspect, the compounds can be used to treat diseases of the central nervous system. Examples of diseases that can be treated with the compounds described herein include, but are not limited to, anxiety, autism, depression, sexual dysfunction, hypertension, migraine, Alzheimer's disease, Huntington's disease, obesity, emesis, psychosis, analgesia, schizophrenia, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, and Tourette's syndrome.

5. Use of Radiolabeled Compounds

It will be appreciated that any of the compounds described herein can also be used as imaging agents or diagnostic agents when labeled with a radionuclide, or fluorescent label. For example, a modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Example A Single Methamphetamine Administration Rapidly Decreases Vesicular Dopamine Uptake*

a) Materials and Methods

(1) Animals

Male Sprague Dawley rats (280-330 g; Simonsen Laboratories, Gilroy, Calif., USA) were maintained under controlled lighting and temperature conditions, with food and water provided ad libitum. Rats were sacrificed by decapitation. All rats were housed in groups of 8 in plastic cages the day prior to the experiment. Mean rectal temperature was determined before drug administration by use of a digital thermometer (Physiotemp Instruments Inc., Clifton, N.J.) where indicated. To restore hyperthermia in treated groups, cages were placed on a heating pad (environmental temperature 28.5° C.). Blockade of hyperthermia was accomplished by placing cages on ice (environmental temperature ˜6° C.). Where indicated, mean rectal temperatures for all treated rats were assessed 10 min prior to drug treatment and again prior to decapitation. All experiments were conducted in accordance with National Institutes of Health guidelines for the care and use of laboratory animals.

(2) Drugs and Radioligands

(+/−) METH hydrochloride was supplied by the National Institute on Drug Abuse (Bethesda, Md., USA). SCH23390 and eticlopride were purchased from Sigma Chemicals (St. Louis Mo., USA). 7,8-[³H]DA (47 Ci/mmol) was purchased from Amersham Life Sciences (Arlington Heights, Ill., USA).

(3) Preparation of Rat Striatal Synaptic Vesicles

Synaptic vesicles were obtained from synaptosomes prepared from rat striatum as described previously (Fleckenstein et al., 1997). Synaptosomes were resuspended and homogenized in cold distilled deionized water. Osmolarity was restored by addition of N-(2-hydroxyethyl)-piperazine-N′-2-ethanesulfonic acid (HEPES) and potassium tartrate 25 mM and 100 mM (final concentrations; pH 7.5 at 4° C.), respectively. Samples were centrifuged for 20 min at 20,000×g (4° C.) to remove lysed synaptosomal membranes. MgSO₄ (1 mM, final concentration) was added to the supernatant, which was then centrifuged for 45 min at 100,000×g (4° C.). The resulting vesicular pellet was resuspended in ice-cold wash buffer (see below) at a concentration of 50 mg/ml (original tissue wet weight) for striatal tissue.

(4) Vesicular [³H]DA Uptake

Vesicular [³H]DA uptake was performed by incubating 100 μL of the resuspended vesicular pellet at 30° C. for 3 min in assay buffer (final concentration in mM: 25 HEPES, 100 potassium tartrate, 1.7 ascorbic acid, 0.05 EGTA, 0.1 EDTA, 2 ATP-Mg²⁺, pH 7.5 at 28.5° C.) in the presence of [³H]DA (30 nM final concentration except in linetic analyses wherein 0.8-10 μM [³H]DA was used). The reaction was terminated by addition of 1 ml cold wash buffer (assay buffer containing 2 mM MgSO₄ substituted for the ATP-Mg²⁺, pH 7.5 at 4° C.) and rapid filtration through Whatman GF/F filters soaked previously in 0.5% polyethylenimine. Filters were washed three times with ice-cold wash buffer using a Brandel filtering manifold (Brandel, Gaithersburg, Md.). Radioactivity trapped in filters was counted using a liquid scintillation counter. Nonspecific values were determined by measuring vesicular [³H]DA uptake at 4° C. in wash buffer (i.e. no ATP present).

(5) Statistical Analysis

Statistical analyses among 3 or more groups were performed using an ANOVA followed by a Fisher PLSD post-hoc comparison. Analysis among two groups was conducted using a paired Student's T-test. Differences were considered significant if probability of error was less than 5%.

b) Results

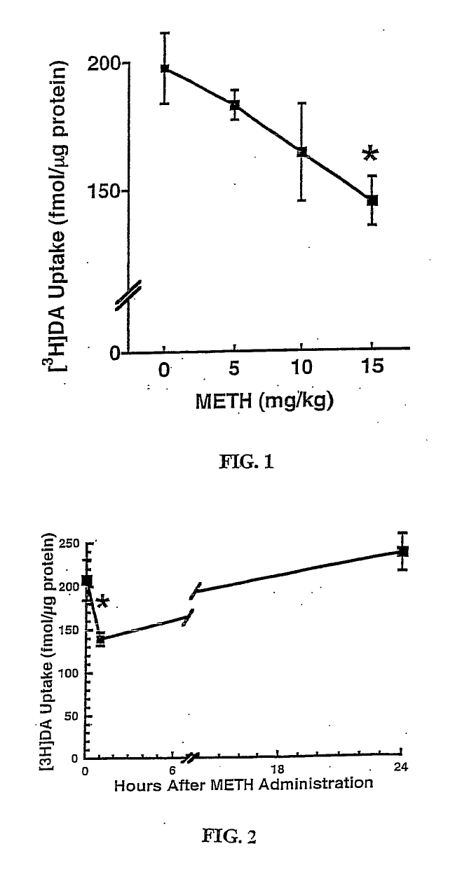

In accordance with a previous report (Brown et al., 2001a), a single METH administration (15 mg/kg, s.c.) rapidly (within 1 h) decreased vesicular [³H]DA uptake as assessed in vesicles purified from the striata obtained from treated rats (FIG. 1). This decrease was attributable to a decrease in the Vₘₐₓ, with little change in the Kₘ of vesicular [³H]DA uptake (in fmol/μg protein/min and nM: 797 and 161 after saline-treatment vs. 607 and 187 after a single 15 mg/kg METH injection, respectively) as assessed as described in Materials and Methods. This effect was dose-related with a dose of 15 mg/kg affecting a 25% decrease in vesicular [³H]DA uptake (FIG. 1). Higher doses were not administered due to increased mortality. The deficit in vesicular [³H]DA uptake recovered by 24 h after treatment (FIG. 2).

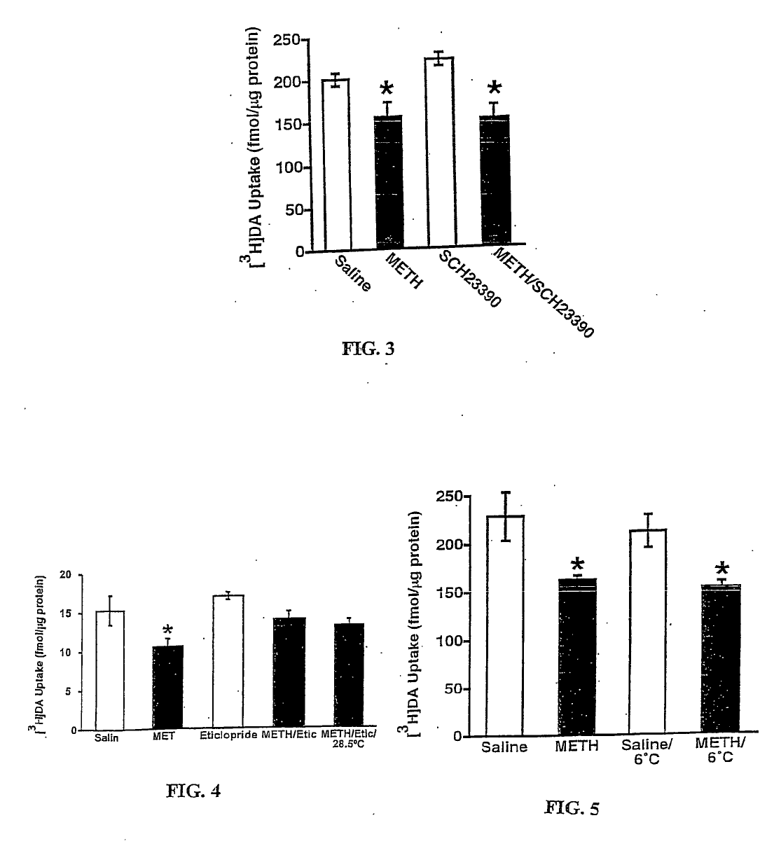

To examine the role of DA D₁ receptors in the METH-induced decrease in vesicular [³H]DA uptake, the D₁ antagonist, SCH23390 (0.5 mg/kg, i.p) was administered 15 min prior to METH treatment. This dose was selected based on previous studies demonstrating that it prevented cocaine-induced increases in D₁-associated parameters such as locomotor activity (Baker et al., 1998) and neuropeptide immunoreactivity (Alburges and Hanson, 1999; Alburges et al., 2000). Data presented in FIG. 3 demonstrate that a single METH injection decreased vesicular [³]DA uptake by 23%; an effect that was not prevented by SCH23390 pretreatment.

The role of DA D₂ receptors in the METH-induced decrease in vesicular [³H]DA uptake was investigated by administering the D₂ antagonist, eticlopride (0.5 mg/kg, i.p.;), 15 min prior to METH treatment. This dose and time point was selected based on previous studies demonstrating its effectiveness at preventing cocaine-induced increases in vesicular DA uptake (Brown et al., 2001b). Data presented in FIG. 4 demonstrate that this pretreatment attenuated the METH-induced deficit. In addition to attenuating the MTH-induced decrease in vesicular uptake, eticlopride pretreatment attenuated the hyperthermia caused by METH (i.e., core body temperatures increased from 36.9±0.1° C. to 40.1±0.1° C. in METH-treated rats vs. 36.8±0.1° C. to 38.4±0.1° C. in METH-treated rats pretreated with eticlopride). Neither saline nor eticlopride pretreatment per se altered rectal temperatures (data not shown). Because METH-induced increases in core body temperature have been implicated in the dopaminergic deficits induced by high-dose METH treatment (Bowyer et al., 1992; Bowyer et al., 1994; Albers and Sonsalla, 1995), the role of this attenuation was investigated. Specifically, some of the eticlopride-pretreated rats were exposed to a warmer ambient temperature (28.5° C.) upon METH treatment in order to maintain hyperthermia. This manipulation resulted in body temperatures of 39.8±0.1 DC; a value similar to that observed after METH treatment in animals exposed to the 24° C. ambient environment. Results presented in FIG. 4 demonstrate that the ability of eticlopride to attenuate the decrease in vesicular [³H]DA uptake induced by METH was not reversed by restoring hyperthermia in the eticlopride/METH-treated rats (40.1±0.1° C.).

To investigate further the role of hyperthermia in mediating the METH-induced decrease in vesicular [³H]DA uptake, rats were treated with either saline or METH and hyperthermia was attenuated by placing these animals in a cool environment (6° C.). Data presented in FIG. 5 demonstrate that attenuation of hyperthermiaper se (36.7±0.1° C. to 40.5±0.1° C. in METH-treated rats vs. 36.8±0.1° C. vs. 38.1±0.1° C. in METH-treated rats exposed to the cool environment) did not prevent the METH-induced decrease in vesicular [³H]DA uptake.

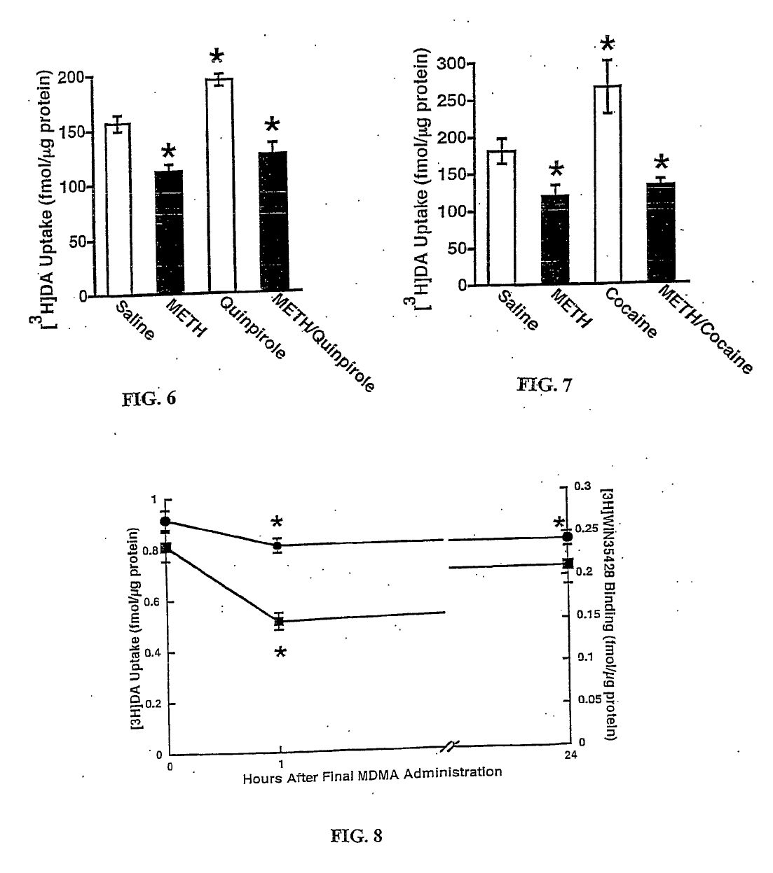

It has been demonstrated previously that administration of either the D₂ DA receptor agonist, quinpirole, or the plasmalemmal DA reuptake inhibitor, cocaine, increases vesicular [³H]DA uptake (Brown et al., 2001a,b). Results presented in FIGS. 6 and 7 demonstrate that neither treatment increased [³H]DA uptake when rats were concurrently treated with METH.

2. Example Methylenedioxymethamphetamine Decreases Plasmalemmal and Vesicular Dopamine Transport: Mechanisms and Implications for Neurotoxicity*

a) Experimental Procedures

(1) Materials

3,4-MDMA hydrochloride and (−)cocaine hydrochloride were generously supplied by the National Institute on Drug Abuse (Bethesda, Md.). (−)-Eticlopride hydrochloride, pargyline hydrochloride, α-methyl-p-tyrosine hydrochloride (αMT), and NPC15347 (S-2,6-Diamino-N-[[1-oxotridecyl)-2-piperidinyl]methyl]-hexanamide dihydrochloride) were purchased from Sigma (St. Louis, Mo.). Ro31-7549 (2-[1-3(Aminopropyl)indol-3-yl]-3(1-methylindol-3-yl)maleimide, acetate) was purchased from Calbiochem (San Diego, Calif.). [7,8-3H]DA (49 Ci/mmol) was purchased from Amersham Pharmacia Biotech (Arlington Heights, Ill.). [N-methyl-³H]WIN35428 (84.5 Ci/mmol) was purchased from New England Nuclear (Boston, Mass.). α-[2-³H]dihydrotetrabenazine ([³H]DHTBZ; 20 Ci/mmol) was purchased from American Radiolabeled Chemicals (St. Louis, Mo.). Tetrabenazine was kindly donated by Drs. Jeffrey Erickson, Helene Varoqui (Louisiana State University Health Sciences Center, New Orleans, La.), and Erik Floor (University of Kansas, Lawrence, Kans.).

(2) Animals

Male Sprague-Dawley rats (270-350 g; Simonsen Laboratories, Gilroy, Calif.) were maintained under controlled light and temperature conditions, with food and water provided ad libitum. On the day of the experiment, rats were housed in groups (8 rats/group) in plastic cages and were maintained in an ambient temperature of 24° C. Where indicated in figure legends, some cages were placed in a cool environment (ambient temperature 6° C.) upon treatment with MDMA or saline to manipulate body temperature (i.e., to prevent the hyperthermia caused by MDMA treatment). Core (rectal) body temperatures were recorded using a digital thermometer (Physiotemp Instruments, Clifton, N.J.) in all experiments in which ambient temperature was manipulated. For experiments in which rats received multiple administrations of MDMA, rectal temperatures were recorded immediately before the first MDMA or saline administration (t=0 h) and every h thereafter (t=0-7 h). Drugs were administered as indicated in the legends of the appropriate figures, and doses were calculated as the respective free bases. All procedures were conducted in accordance with National Institutes of Health Guidelines for the Care and Use of Laboratory Animals.

(3) [³H]DA Uptake via Plasmalemmal Transporters and [3H]WIN35428 Binding

Uptake of [³H]DA was determined in striatal synaptosomes prepared according to the method described by Kokoshka et al. (1998). Briefly, fresh striatal tissue was homogenized in cold 0.32 M sucrose and centrifuged (800×g for 12 min; 4° C.). The supernatant (S1) was then centrifuged (22,000×g for 15 min; 4° C.), and the resulting pellet (P2) was resuspended in ice-cold modified Kreb's buffer (in mM: 126 NaCl, 4.8 KCl, 1.3 CaCl₂, 16 sodium phosphate, 1.4 MgSO₄, 11 dextrose, 1-ascorbic acid, pH 7.4). Assays were conducted in Kreb's buffer. Each assay tube contained synaptosomal tissue (i.e., resuspended P2 obtained from 1.5 mg of original wet weight striatal tissue) and 1 μM pargyline. Nonspecific values were determined in the presence of 100 μM cocaine. After preincubation of assay tubes for 10 min at 37° C., assays were initiated by the addition of [³H]DA (0.5 nM final concentration). Samples were incubated at 37° C. for 3 min. Samples were then filtered through Whatman GF/B filters (Brandel, Gaithersburg, Md.) soaked previously in 0.05% polyethylenimine. Filters were washed rapidly 3 times with 3 ml of ice-cold 0.32 M sucrose using a Brandel filtering manifold. Radioactivity trapped in filters was counted using a liquid scintillation counter. Remaining resuspended P2 samples were assayed for protein concentrations according to the method of Lowry et al. (1951). In MDMA preincubation experiments, samples were preincubated with 10 μM MDMA for 30 min at 37° C. After 30 min, resuspended P2 fractions were “washed” by centrifugation (22,000×g for 15 min; 4° C.). The resulting pellet (P3) was then resuspeded in ice-cold Kreb's buffer, and once again centrifuged (22,000×g for 15 min; 4° C.) to obtain a P4 pellet that was subsequently resuspended and assayed. [³H]WIN35428 binding (0.5 nM final concentration) was conducted in phosphate-buffered 0.32 M sucrose, pH 7.4, with synaptosomes obtained from 2 mg (original wet weight) of striatal tissue per reaction tube, and samples were incubated on ice for 2 hr. Samples were then filtered through Whatman GF/B filters (Brandel, Gaithersburg, Md.) soaked previously in 0.05% polyethylenimine. Filters were washed rapidly 3 times with 3 ml of ice-cold 0.32 M sucrose using a Brandel filtering manifold. Radioactivity trapped in filters was counted using a liquid scintillation counter. Remaining resuspended P2 samples were assayed for protein concentrations according to the method of Lowry et al. (1951).

(4) [³H]DA Uptake via Vesicular Monoamine Transporters and [³H]DHTBZ Binding

Synaptic vesicles were obtained from synaptosomes prepared from rat striatum as described above. Synaptosomes were resuspended and homogenized in cold distilled deionized water. Osmolarity was restored by addition of HEPES and potassium tartrate 245 and 100 mM (final concentrations; pH 7.5), respectively. Samples were centrifuged for 20 min at 20,000×g (4° C.) to remove lysed synaptosomal membranes. MgSO₄ (1 mM, final concentration) was added to the supernatant, which was then centrifuged for 45 min at 100,000×g (4° C.). The resulting vesicular pellet was resuspended in wash buffer at a concentration of 50 mg/ml (original tissue weight). Based on published reports using similar protocols for vesicle preparation (Kadota and Kadota, 1973; Teng et al., 1997), we believe vesicles isolated in these studies to be of the small synaptic vesicle size (˜50 nM), the predominant type found in dopaminergic terminals in the striatum (Nirenberg et al., 1997). Vesicular [³H]DA uptake was performed by incubating 100 μl of synaptic vesicle samples (˜2.5 μg of protein) at 30° C. for 3 min in assay buffer (final concentration in mM: 25 HEPES, 100 potassium tartrate, 1.7 ascorbic acid, 0.05 EGTA, 0.1 EDTA, 2 ATP-Mg²⁺, pH 7.5) in the presence of [³H]DA (30 nM final concentration). The reaction was terminated by addition of 1 ml of cold wash buffer (assay buffer containing 2 mM MgSO₄ substituted for the ATP-Mg²⁺, pH 7.5) and rapid filtration through Whatman GF/F filters soaked previously in 0.5% polyethylenimine. Filters were washed three times with cold wash buffer using a Brandel filtering manifold. Radioactivity trapped in filters was counted using a liquid scintillation counter. Nonspecific values were determined by measuring vesicular [³H]DA uptake at 4° C. in wash buffer. Binding of [³H]DHTBZ was performed as described by Teng et al. (1998). Briefly, 200 μl of the synaptic vesicle preparation (˜6 μg of protein) was incubated in wash buffer in the presence of [³H]DHTBZ (2 nM final concentration) for 10 min at 25° C. The reaction was terminated by addition of 1 ml of cold wash buffer and rapid filtration through Whatman GF/F filters soaked in 0.5% polyethylenimine. Filters were washed three times with ice-cold wash buffer. Nonspecific binding was determined by coincubation with 20 μM tetrabenazine. All protein concentrations were determined by a BioRad protein assay (Bio-Rad, Richmond, Calif.).

(5) Dopamine Content

On the day of the assay, frozen tissue samples were thawed, sonicated for 3-5 s in tissue buffer (0.05 M sodium phosphate/0.03 M citric acid buffer with 15% methanol (v/v); pH 2.78), and centrifuged for 15 min at 22,000×g. Tissue pellets were retained and protein determined according to the method of Lowry et al. (1951). The supernatant was centrifuged a second time for 15 min at 22,000×g. 20 μl of supernatant were injected onto a high performance liquid chromatograph system coupled to an electrochemical detector (+0.73 V) for separation and quantitation of dopamine levels using the method of Chapin et al. (1986).

(6) Statistics

Statistical analyses were performed using an ANOVA followed by a Fisher's protected least-significant difference post hoc comparison or Student's t test as indicated. Differences were considered significant if probability of error was p≦0.05.

b) Results

Results presented in FIG. 8 confirm previous reports that multiple high-dose administrations of MDMA rapidly (within 1 h) decrease plasmalemmal DA uptake function, as assessed in synaptosomes prepared from treated rats. This deficit represents a decrease in Vₘₐₓ (2388 and 1410 fmol/μg/5 min for saline- and MDMA-treated rats, respectively), while transporter Kₘ was virtually unaffected (99.6 vs. 98.9 nM for saline- and MDMA-treated rats, respectively; Metzger et al., 1998). This deficit was reversed 24 h after drug treatment. In contrast, binding of the DAT ligand, WIN35428, was only slightly reduced (i.e., by 10%) 1 h after treatment: this deficit persisted 24 h after drug treatment.

Multiple administrations of MDMA (4×10 mg/kg; 2-h intervals; s.c.) to rats typically increases core body temperature by approximately 2-4° C. Previous studies have demonstrated that such hyperthermia contributes to the deficit in plasmalemmal DA uptake caused by multiple administrations of METH (Metzger et al., 2000). Hence, the role of body temperature in the reduction in plasmalemmal DA uptake induced by multiple administrations of MDMA was assessed by preventing the MDMA-induced increase in body temperature. Upon administration of MDMA, some rats were exposed to an ambient temperature of 6° C. for the duration of the experiment (in order to maintain normothermic body temperature), while other MDMA-treated rats remained exposed to room temperature (24° C.) to allow hyperthermia to occur. As shown in FIG. 9A, attenuation of MDMA-induced hyperthermia did not prevent the rapid decrease in [³H]DA uptake induced by multiple administrations of MDMA. In this experiment, WIN35428 binding was not affected by either MDMA administration or by manipulating body temperatures (data not shown). Corresponding rat core body temperatures are shown in FIG. 9B.

In addition to demonstrating a role for hyperthermia, previous studies have shown that DA contributes to the deficit in DAT function caused by multiple administrations of METH (Metzger et al., 2000). Hence, the role of DA in the reduction of plasmalemmal DA uptake induced by multiple administrations of MDMA was assessed by depleting striatal DA levels by administering the tyrosine hydroxylase inhibitor, αMT, prior to MDMA treatment. αMT (150 mg/kg; i.p.) was injected 5 and 1 h prior to, and 3 h after, the first injection of MDMA. Striatal DA levels were greatly reduced by αMT pretreatment (55.0±5.0 vs. 10.1±2.0 pg/1 μg protein for saline- vs. αMT-treated rats, respectively; p≦0.05). As demonstrated in FIG. 10A, pretreatment with αMT did not affect the MDMA-induced decrease in DAT activity. In this experiment, WIN35428 binding was decreased by 18% after MDMA treatment: αMT pretreatment did not prevent this deficit (FIG. 10B).

In order to elucidate the mechanism(s) whereby MDMA decreases DAT function in vitro, striatal synaptosomes were incubated with MDMA (10 μM) for 30 min at 37° C. A similar incubation paradigm demonstrated that this in vitro model appears to model some effects of METH treatment on DAT in vivo (Sandoval et al., 2001). Results presented in FIG. 11 demonstrate that MDMA treatment also decreases DA uptake in vitro with a magnitude similar to that observed after multiple in vivo administrations of MDMA (i.e., 35-55%; compare with FIGS. 8-10). Pretreatment with the protein kinase C (PKC) inhibitor, NPC15437, attenuated the MDMA-induced deficit caused by in vitro incubation with MDMA (FIG. 11A). Moreover, pretreatment with another selective PKC inhibitor, Ro31-7549, attenuated the MDMA-induced deficit in vitro as well (FIG. 11B). Incubation of synaptosomes with MDMA had no effect on WIN35428 binding.

Results presented in FIG. 12 demonstrate that not only does MDMA treatment rapidly diminish plasmalemmal DA uptake, but striatal vesicular DA uptake as well. Specifically, multiple MDMA administrations rapidly decreased vesicular uptake, as assessed in vesicles purified from striata of treated animals. This deficit partially recovered 24 h after drug treatment. In addition, MDMA treatment reduced binding of the VMAT-2 ligand, [³H]DHTBZ, both 1 and 24 h after treatment.

Results presented in FIGS. 13A and 13B show that similar to the MDMA-induced effects on plasmalemmal DA transport, hyperthermia did not contribute to the drug-induced decrease in vesicular DA uptake or [³H]DHTBZ binding since its prevention did not attenuate these deficits. Corresponding rat core body temperatures are shown in FIG. 13 C.

In the next experiment, the role of DA in the MDMA-induced decrease in vesicular DA uptake was assessed. Because depletion of DA resulting from αMT treatment increases vesicular DA uptake per se (Brown et al., 2001), the tyrosine hydroxylase inhibitor was not employed in this experiment. Instead the role of D2 receptors was determined using the D2 antagonist, eticlopride. Data presented in FIG. 14 demonstrate that administration of eticlopride (0.5 mg/kg, i.p.) 15 min before each MDMA injection attenuated the MDMA-induced decrease in vesicular DA uptake.

3. Example Methamphetamine Rapidly Decreases Mouse Vesicular Dopamine Uptake: Role of Dopamine Receptors and Hyperthermia

a) Materials and Methods

(1) Experimental Animals

Male CF-1 mice (25-36 g; Charles River; Portage, Mich.) were housed in groups of 4 in plastic cages, maintained under conditions of controlled temperature of 24° C. on a 14/10 hr light/dark cycle, unless otherwise specified in figure legends. Food and water were provided ad libitum. On the day of the experiment, mice were housed in-groups of eight in plastic cages. Core (rectal) body temperatures were determined using a digital rectal thermometer (Physiotemp Instruments, Clifton, N.J.). Mice were sacrificed by decapitation. All procedures were conducted in accordance with approved National institutes of Health guidelines.

(2) Drugs and Chemicals

Methamphetamine hydrochloride and methylenedioxymethamphetamine hydrochloride (MDMA; “ecstasy”) were supplied generously by the National Institute on Drug and Abuse. Methylphenidate hydrochloride was obtained from Ciba Geigy (Summit, N.J.). [7,8-³H]Dopamine (47-50 Ci/mmol) was purchased from Amersham Life Sciences (Arlington Heights, Ill.) and [2-³H]DHTBZ (20 Ci/mmol) was purchased from American Radiolabeled Chemicals Inc. (St. Louis, Mo., USA). Tetrabenazine (TBZ) was kindly donated by Drs. Jeffrey Erickson and Helene Varoqui, (Louisiana State University Health Sciences, New Orleans, La., USA). SCH23390 and eticlopride were purchased from Sigma (St. Louis, Mo.). Drugs were administrated as indicated in the legends of appropriate figures, and doses were calculated as the respective free bases. Drugs were dissolved in 0.9% saline.

(3) Preparation of Mouse Striatal Synaptic Vesicles

Synaptic vesicles were obtained from synaptosomes prepared from mouse striatum as described previously. (A. E. Fleckenstein, et al., J. Pharmacol Exp Ther. (1997) 282:834-838). Briefly, fresh tissue was homogenized in ice-cold 0.32 M sucrose. The homogenate was centrifuged (800×g for 12 min; 4° C.), and the supernatant (S1) was carefully removed and centrifuged (22,000×g for 15 min; 4° C.) to obtain the synaptosomal-containing pellet (P2). The resulting P2 were resuspended and homogenized in ice-cold distilled deionized water. Osmolarity was restore by addition 25 mM HEPES and 100 mM potassium tartrate (final concentration; pH 7.5 at 4° C. respectively). Samples were centrifuged for 20 min at 20,000×g:4° C. The resultant S3 removed and MgSO₄ added (final concentration of [1 mM] pH 7.5 at 4° C.) and centrifuged at 100,000×g for 45 min. The final P4 were resuspended at 50 mg/ml (original tissue wet weight).

(4) Vesicular [³H]dopamine Uptake and [³H]DHTBZ Binding

Vesicular [³H]dopamine uptake was performed by incubating 100 μl of synaptic vesicle samples (˜2.5 μg protein) at 30° C. for 3 min in assay buffer (final concentration) in mM: 25 Hepes, 100 potassium tartrate, 1.7 ascorbic acid, 0.05 EGTA, 0.1 EDTA, 2 ATP-Mg²⁺, pH 7.5], 30° C. in the presence of [³H]dopamine (30 nM final concentration). The reaction was terminated by addition of 1 ml cold wash buffer (assay buffer containing 2 mM MgSO₄ substituted for the ATP-Mg²⁺, pH 7.5 at 4° C.) and rapid filtration though Whatman GF/F filters soaked previously in 0.5% polyethylenimine. Filters were washed three times with ice-cold wash buffer using a Brandel filtering manifold. Radioactivity trapped in filters was counted using a liquid scintillation counter. Nonspecific values were determined by measuring vesicular [³H]dopamine uptake in wash buffer (i.e., no ATP present) at 4° C.

Binding of [³H]DHTBZ was performed essentially as described by Brown et al. (2001a and 2001b) Briefly, 200 μl of the synaptic vesicles preparation (˜6 μg of protein) was incubated in wash buffer in the presence of [³H]DHTBZ (2 nM final concentration) for 10 min at 25° C. The reaction was terminated by addition of 1 ml cold wash buffer and rapid filtration through Whatman GF/P filters soaked in 0.5% polyethylenimine. Filters were washed three times with ice-cold wash buffer. Nonspecific binding was determined by co incubation with 20 μM TBZ. All protein concentrations were determined by Bio-Rad protein assay (Bio-Rad Inc.)

(5) Preparation of Striatal Subcellular Fractions

Fresh striatal tissue was homogenized in ice-cold 0.32 M sucrose and centrifuged (800×g for 12 min: 4° C.). The resulting supernatant (S1) was then centrifuged (22,000×g for 10 min: 4° C.), and the pallets (P2; whole synaptosomal fraction (plasmalemmal membrane plus vesicular subcellular fraction) were resuspended in cold distilled deionized water at a concentration of 50 mg/ml (original wet weight of tissue). Resuspended tissue was aliquoted into two test tubes. One aliquot was centrifuged (22,000×g for 10 min; 4° C.) to separate plasmalemmal membranes from the synaptic vesicle-enriched fraction. The resulting supernatant (S3) contained the vesicular subcellular fraction of interest, and the pellets (P3; plasmalemmal membrane fraction) were resuspended in cold distilled deionized water.

(6) Western Blots Analysis

VMAT-2 antibody was kindly donated by Dr. John Haycock at the Louisiana State University, New Orleans, La. (Antibody can be purchased from Chemicon, Temecula, Calif. and the reagent # is AB1767). Binding of VMAT-2 antibody was performed using 60 μl of whole synaptosomal, plasmalemmal membrane or vesicle subcellular fractions. Samples were added to 20 μl of loading buffer (final concentration: 2.25% SDS, 18% glycerol, 180 mM Tris Base (pH 6.8), 10% β-mercaptoethanol and bromophenol blue). Approximately 60 μg P2, 40 μg protein P3, and 20 μg protein S3 were loaded per well in a 10% SDS-polyacrylamide gel. Following electrophoresis, samples were transferred to polyvinylidene difluoride hybridization transfer membrane (New England Nuclear (NEN), Boston, Mass.). All subsequent incubation steps were performed at room temperature while shaking. Each membrane was first blocked for 2 h in 100 ml of Tris buffer saline with tween (TBST; 250 mM NaCl, 50 mM tris pH 7.4 and 0.05% tween 20) containing 5% nonfat dry milk. Each membrane was then incubated with anti-VMAT-2 antibody (1:4000 dilution) in 13 ml of TBST with 5% milk for 1 h and then washed 5 times (2×1 min wash: 3×5 min wash) in 70 ml TBST with 5% milk. The membranes then were incubated for 1 h with the goat F (ab′)₂ anti-rabbit immunoglobulin antibody (Biosource International, Camarillo, Calif.) at a 1:2000 dilution in TBST with 5% milk. This secondary antibody had been affinity-isolated, preabsorbed with human immunoglobulin, and conjugated with horseradish peroxidase. The membranes were then washed 5 times (2×1 min wash: 3×5 min) with 70 ml TBST, and then developed with Renaissance Western Blot Chemiluminescence's Reagent Pus (NEN, Boston, Mass.), according to manufacturer specification. Multiple exposures of blots were obtained to e4 nsure development within the linear range of the film (Kodak Biomax MR). Bands on blots were quantified by densitometry measuring net intensity (the sum of the background-subtracted pixel values in the band area) using Kodak 1D image-analysis software.

(7) Striatal Dopamine Content

On the day of the assay, frozen striatal samples were sonicated 3-5 s in cold tissue buffer (0.05 M sodium phosphate/0.03 M citric acid buffer with 15% methanol (v/v); pH 2.5), and centrifuged for 15 min at 22,000×g. Resulting tissue pellets were retained and protein was determined according to the method of Lowry et al. (O. H. Lowry, et al., J. Biol. Chem. 193 (1951) 265-275). The resulting supernatant was centrifuged a second time for 10 min at 20,000×g. Twenty μl of supernatant were injected onto a high performance liquid chromatograph system coupled to an electrochemical detector (+0.73 V) for separation and quantification of dopamine levels using the method of Chapin et al. (D. S. Chapin, et al., Curr. Separations 7 (1986) 68-70).

(8) Data Analysis

Statistical analyses were performed using either a Student's T-test or analysis of variance followed by Fisher-protected least significant difference multiple comparisons test. Differences among groups were considered significant if the probability of error was less than 5%.

b) Results

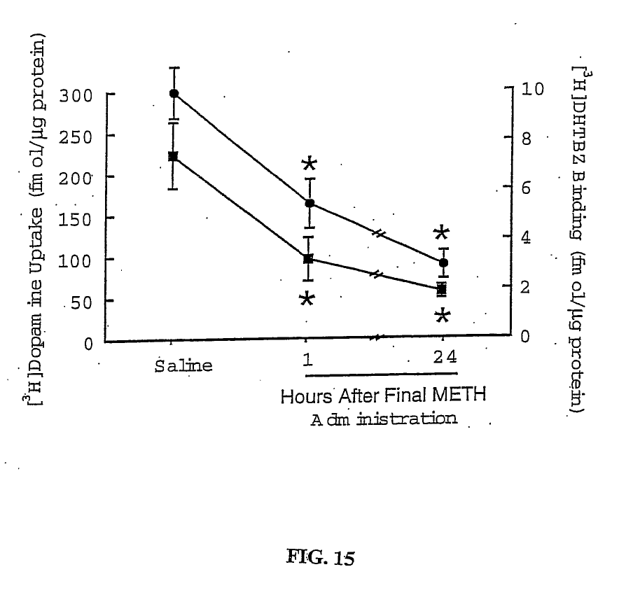

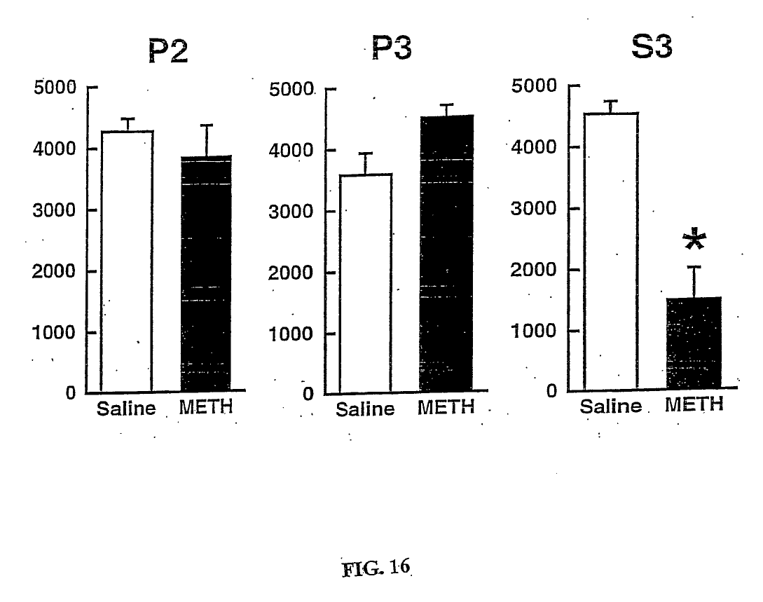

Results presented in FIG. 15 demonstrate that multiple high-dose injections of methamphetamine (4 injections, 10 mg/kg/injection, s.c., 2-h intervals) rapidly decreased both vesicular [³H]dopamine uptake and [³H] DHTBZ binding, as assessed 1 and 24 h after treatment. The deficit observed 1 h after treatment reflected a redistribution of VMAT-2 immunoreactivity among subcellular fractions (FIG. 16). Specifically, methamphetamine had little effect on total VMAT-2 immunoreactivity in synaptosomes prepared from treated mice. However, upon osmotic lyses and subsequent fractionating of synaptosomes into the purified vesicular fraction and a remaining membrane-associated fraction, a redistribution was observed such that methamphetamine treatment decreased VMAT-2 immunoreactivity in the vesicle preparation, while increasing it in a corresponding membrane-associated fraction.

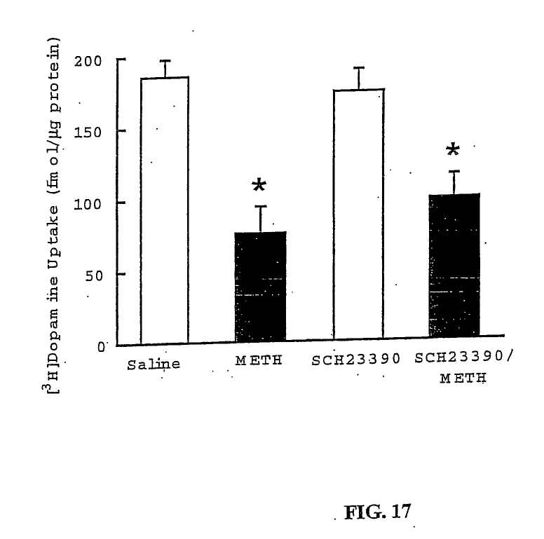

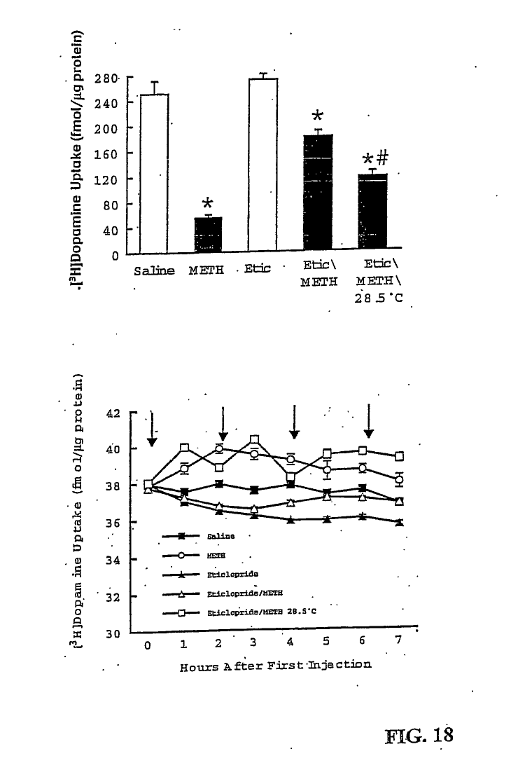

Results presented in FIG. 17 demonstrate that pretreatment with the D₁ receptor antagonist, SCH23390 (2 mg/kg, i.p.), did not prevent the decrease in vesicular dopamine uptake caused by methamphetamine treatment. In contrast, pretreatment with the D₂ receptor antagonist, eticlopride (2 mg/kg, i.p.) attenuated this decrease (FIG. 18a). In addition, eticlopride pretreatment attenuated the increase in core body temperature caused by methamphetamine treatment (FIG. 18b). Maintenance of hyperthermia in the mice treated with eticlopride and methamphetamine (i.e., by placing mice in a 28° C. environment instead of the ambient environment of 23° C.) attenuated the ability of eticlopride to prevent the methamphetamine-induced decrease in vesicular dopamine uptake (FIGS. 18a and 18b).

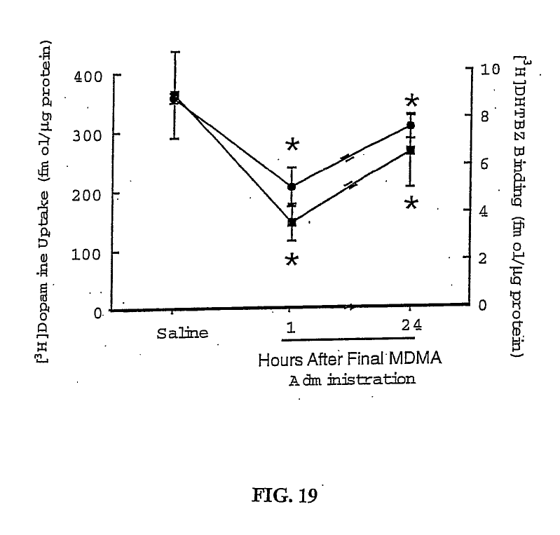

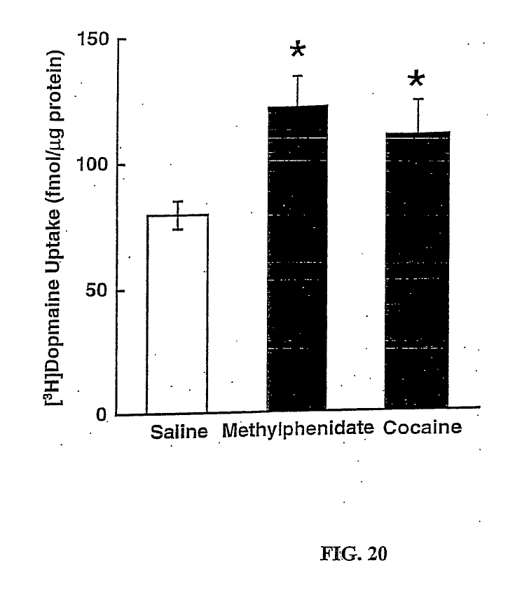

To investigate whether the METH-induced decrease was unique to this amphetamine analog, effects of MDMA were investigated. Results presented in FIG. 19 demonstrate that multiple administrations of MDMA (4 injections, 10 mg/kg/injection, s.c.) rapidly decreased vesicular dopamine uptake. In contrast to effects of methamphetamine, this decrease was largely reversed 24 h later (compare FIGS. 15 and 19). Interestingly, this MDMA regimen caused minimal (13%) persistent dopaminergic deficits as assessed by measuring dopamine tissue content 7 days after drug treatment (113±3 and 99±3 pg/μg protein, n=9 per group).

Noteworthy is the finding that not all psychostimulants decrease vesicular dopamine uptake in the purified vesicular fractions under study. Specifically, a single injection of methylphenidate (10 mg/kg, s.c.) or cocaine (30 mg/kg, i.p.) increased vesicular dopamine uptake as assessed in vesicles prepared 1 h after treatment (FIG. 20).

4. Example Differential Trafficking of the Vesicular Monoamine Transporter-2 by Methamphetamine and Cocaine

a) Materials and Methods

All experiments were conducted in accordance with the NIH Guidelines for the Care and Use of Laboratory Animals. Where indicated, male Sprague-Dawley rats (weighing 280-330 g) received a single injection of cocaine (30 mg/kg i.p.), multiple high-dose injections of methamphetamine (4×10 mg/kg per injection, s.c., 2-h intervals), or saline vehicle (1 ml/kg per injection).

Striatal synaptosomes were prepared from rats decapitated 1 h after treatment as previously described (Fleckenstein et al., 1997). Briefly, striatal tissue was homogenized in cold 0.32 M sucrose and centrifuged (800×g for 12 min; 4° C.). The supernatant (S1) was then centrifuged (22,000×g for 15 min; 4° C.) and the resulting pellet (P2, synaptosomal fraction) was resuspended at 50 mg original wet weight/ml in cold water and a portion saved for western blot analysis. The remainder of the synaptosomal sample was centrifuged for 20 min at 22,000×g (4° C.) to pellet lysed synaptosomal membranes (P3, synaptosomal membrane fraction), which were then resuspended at 50 mg original wet weight/ml and saved for western blot analysis. Prior to resuspension of the plasmalemmal membrane fraction the supernatant (S3, vesicle-enriched fraction) was removed and saved for western blot analysis.

Binding of VMAT-2 antibody was performed using 60 μl aliquots of synaptosomal (P2), synaptosomal membrane (P3), or vesicle-enriched (S3) preparations. Each aliquot was added to 20 μl of loading buffer (final concentration: 2.25% sodium dodecyl sulfate, 18% glycerol, 180 mM Tris base (pH 6.8), 10% β-mercapto-ethanol and bromophenol blue), boiled for 10 min, and loaded on a 10% sodium dodecyl sulfate-polyacrylamide gel. Following electrophoresis, samples were transferred to polyvinlylidene difluoride membrane, blocked with 5% nonfat dry milk in Tris-buffered saline with tween (250 mM NaCl, 50 mM Tris pH 7.4 and 0.05% Tween 20), and probed with the VMAT-2 antibody (provided by J.W.H.). Bound antibody was visualized with HRP-conjugated goat anti-rabbit antibody, and antigen-antibody complexes were visualized by chemiluminescence. Multiple exposures of blots were obtained to ensure development within the linear range of the film. Bands on blots were quantified by densitometry using Kodak 1D image-analysis software.

b) Results

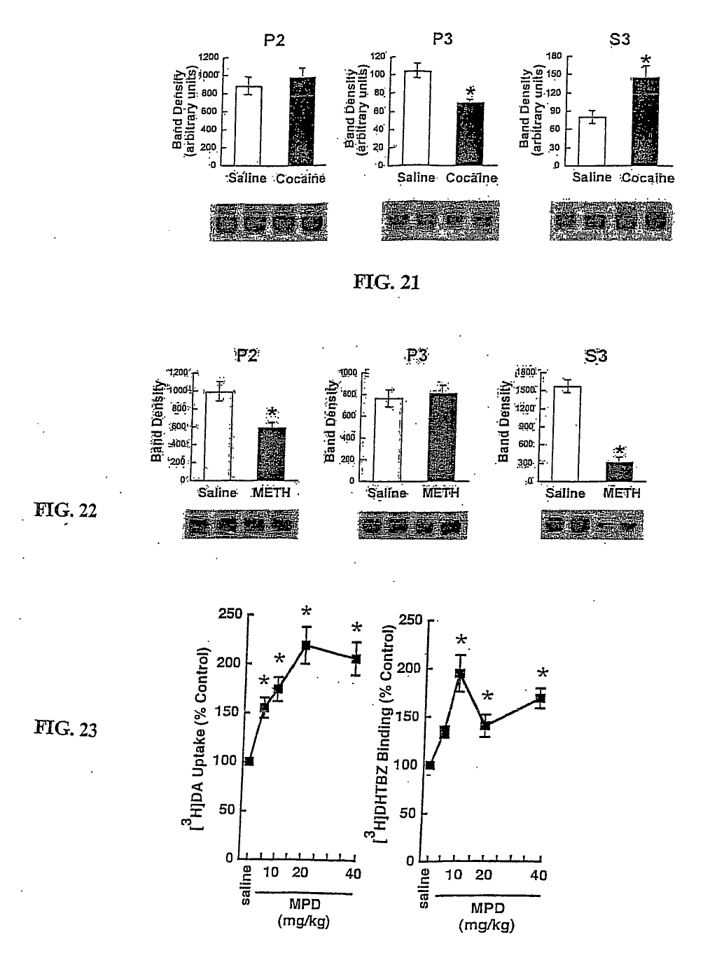

Results presented in FIG. 21 demonstrate that a single injection of cocaine (30 mg/kg; i.p.) increases VMAT-2 immunoreactivity by 80% in the S3 (vesicle-enriched) fraction prepared from the striata of rats sacrificed 1 h after treatment. This increase was concurrent with a 33% decrease in the associated P3 (synaptosomal membrane) fraction, with no difference between P2 (synaptosomal) fractions. Data presented in FIG. 22 demonstrate that 1 h after multiple high-dose administration of methamphetamine (4×10 mg/kg; s.c.), VMAT-2 immunoreactivity in the S3 fraction was decreased by 80% compared to saline-treated controls. This decrease was concurrent with a 40% decrease in the P2 fraction and no difference in the P3 fractions.

5. Example Methylphenidate Redistributes Vesicular Monoamine Transporter-2: Role of Dopamine Receptors

a) Materials and Methods

(1) Animals

Male Sprague-Dawley rats (280-340 g; Simonsen Laboratories, Gilroy, Calif.) were maintained under controlled lighting and temperature conditions, with food and water provided ad libitum. Rats were sacrificed by decapitation using a guillotine. Striata (40-50 mg in weight per rat) were dissected and quickly placed in cold 0.32 M sucrose until tissue was processed (see below for details). All procedures were conducted in accordance with National Institutes of Health Guidelines for the Care and Use of Laboratory Animals and approved by the University of Utah Institutional Animal Care and Use Committee.

(2) Drugs and Chemicals

(±)MDP hydrochloride was supplied by the National Institute on Drug Abuse (Bethesda, Md.). 7,8-[³H]DA (48 Ci/mmol) was purchased from Amersham Life Sciences (Arlington Heights, Ill.) and α-[2-³H]DHTBZ (20 Ci/mmol) was purchased from American Radiolabeled Chemicals Inc. (St. Louis, Mo.). Tetrabenazine (TBZ) was kindly donated by Drs. Jeffrey Erickson, Helene Varoqui (Louisiana State University Health Sciences Center, New Orleans, La.) and Erick Floor (University of Kansas, Kans.). All drugs were administered at 1 ml/kg, as indicated in figure legends. Doses were calculated as the respective free base and drugs were dissolved in 0.9% saline.

(3) Preparation of Striatal Synaptic Vesicles

Synaptosomes prepared from rat striatum as described previously (Fleckenstein et al., 1997). Synaptosomes were then resuspended and homogenized in cold distilled deionized water. Osmolarity was restored by addition of N-(2-hydroxyethyl)-piperazine-N′-2-ethanesulfonic acid (HEPES) and potassium tartrate (final concentration in mM: 25 and 100, respectively; pH 7.5). Samples were centrifuged for 20 min at 20,000×g (4° C.) to remove lysed synaptosomal membranes. MgSO₄ (1 mM, final concentration) was added to the supernatant, which was then centrifuged for 45 min at 100,000×g (4° C.). The resulting vesicular pellet was resuspended in wash buffer (see below) at a concentration of 50 mg/ml (original wet weight of tissue).

(4) Vesicular [³H]DA Uptake and [³H]DHTBZ Binding

Vesicular [³H]DA uptake was performed by incubating 100 μl (˜2.5 μg protein) of synaptic vesicle samples at 30° C. for 3 min in assay buffer (final concentration in mM: 25 HEPES, 100 potassium tartrate, 1.7 ascorbic acid, 0.05 EGTA, 0.1 EDTA, 2 ATP-Mg²⁺, pH 7.5) in the presence of [³H]DA (30 nM final concentration except in kinetic analyses wherein 0.8-10 μM [³H]DA was employed). The reaction was terminated by addition of 1 ml cold wash buffer (assay buffer containing 2 mM MgSO₄ substituted for the ATP-Mg²⁺, pH 7.5) and rapid filtration through Whatman GF/F filters soaked previously in 0.5% polyethylenimine. Filters were washed three times with cold wash buffer using a Brandel filtering manifold. Radioactivity trapped in filters was counted using a liquid scintillation counter. Nonspecific values were determined by measuring vesicular [³H]DA uptake at 4° C. in wash buffer.

Binding of [³H]DHTBZ was performed as described by Teng et al. (1998). Briefly, 200 μl (˜6 μg protein) of the synaptic vesicle preparation was incubated in wash buffer in the presence of [³H]DHTBZ (2 nM final concentration except in kinetic analyses wherein 0.25-500 nM DHTBZ was employed) for 10 m in at 25° C. The reaction was terminated by addition of 1 ml cold wash buffer and rapid filtration through Whatman GF/F filters soaked in 0.5% polyethylenimine. Filters were washed three times with ice-cold wash buffer. Radioactivity trapped in filters was counted using a liquid scintillation counter. Nonspecific binding was determined by coincubation with 20 μM TBZ. All protein concentrations were determined by a Bio-Rad protein assay (Bio-Rad Inc.).

(5) Preparation of Striatal Subcellular Fractions

Fresh striatal tissue was homogenized in ice-cold 0.32 M sucrose and centrifuged (800×g for 12 min; 4° C.). The resulting supernatant (S1) was then centrifuged (22,000×g for 10 min; 4° C.), and the pellets (P2; whole synaptosomal fraction (plasmalemmal membrane plus vesicular subcellular fractions)) were resuspended in cold distilled deionized water at a concentration of 50 mg/ml (original wet weight of tissue). Resuspended tissue was aliquoted into two test tubes. One aliquot was centrifuged (22,000×g for 10 min; 4° C.) to separate plasmalemmal membranes from the synaptic vesicle-enriched fraction. The resulting supernatant (S3) contained the vesicular subcellular fraction of interest, and the pellets (P3; plasmalemmal membrane fraction) were resuspended in cold distilled deionized water.

(6) Western Blot Analysis

VMAT-2 antibody was purchased from Chemicon (Temecula, Calif.; AB1767). Binding of VMAT-2 antibody was performed using 60 μl of whole synaptosomal, plasmalemmal membrane or vesicle subcellular fractions. Samples were added to 20 μl of loading buffer (final concentration: 2.25% SDS, 18% glycerol, 180 mM Tris Base (pH 6.8), 10% α-mercaptoethanol and bromophenol blue). Approximately 60 μg protein of the whole synaptosomal fraction, 40 μg protein of the plasmalemmal membrane fraction or 20 μg protein of the vesicle subcellular fraction was loaded per well in a 10% SDS-polyacrylamide gel. Following electrophoresis, samples were transferred to polyvinylidene difluoride hybridization transfer membrane (New England Nuclear (NEN), Boston, Mass.). All subsequent incubation steps were performed at room temperature while shaking. Each membrane was first blocked for 2 h in 100 ml of tris buffer saline with tween (TBST; 250 mM NaCl, 50 mM tris pH 7.4 and 0.05% tween 20) containing 5% nonfat dry milk. Each membrane was then incubated with anti-VMAT-2 antibody (1:4000 dilution) in 13 ml of TBST with 5% milk for 1 h and then washed 5 times (2×1 min wash; 3×5 min wash) in 70 ml TBST with 5% milk. The membranes then were incubated for 1 h with the goat F(ab′)₂ anti-rabbit immunoglobulin antibody (Biosource International, Camarillo, Calif.) at a 1:2000 dilution in TBST with 5% milk. This secondary antibody had been affinity-isolated, preabsorbed with human immunoglobulin, and conjugated with horseradish peroxidase. The membranes were then washed 5 times (2×1 min wash; 3×5 min wash) with 70 ml TBST, and then developed with the Renaissance Western Blot Chemiluminescence Reagent Plus (NEN, Boston, Mass.), according to manufacturer specification. Multiple exposures of blots were obtained to ensure development within the linear range of the film (Kodak Biomax MR). Bands on blots were quantified by densitometry measuring net intensity (the sum of the background-subtracted pixel values in the band area) using Kodak 1D image-analysis software.

(7) Data Analysis

Statistical analyses among 3 or more groups were performed using an analysis of variance followed by a Fisher PLSD post-hoc comparison. Analyses between 2 groups were conducted using a Student's t-test. Differences were considered significant if probability of error was less than 5%.

b) Results

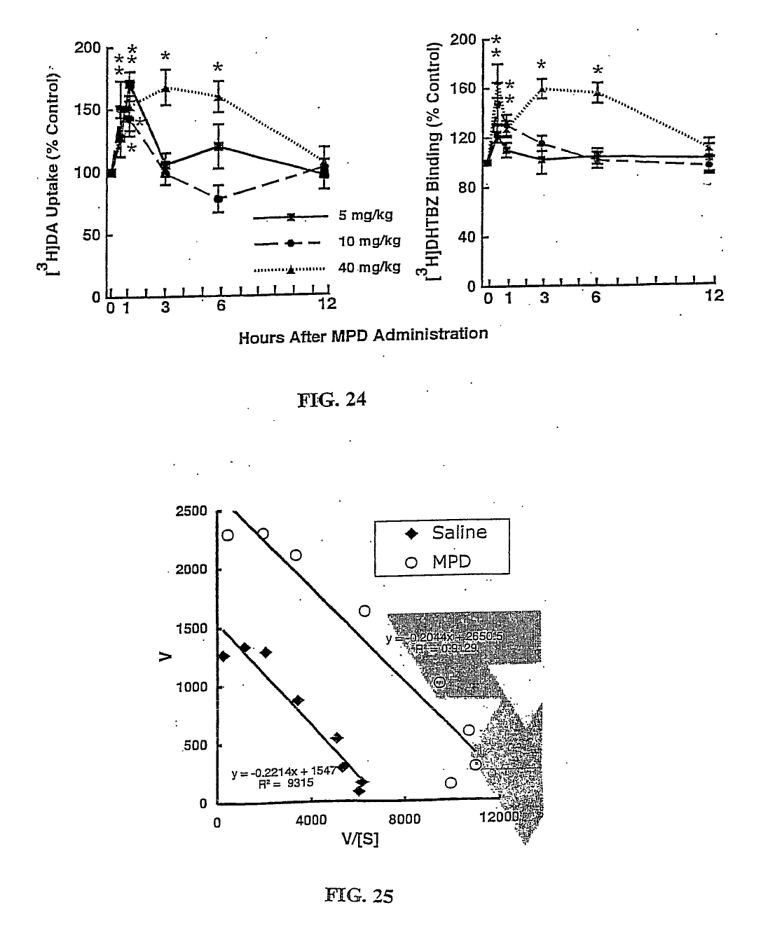

Results presented in FIG. 23A demonstrate that MPD increases vesicular [³H]DA uptake after a single administration of 5, 10, 20, or 40 mg/kg MPD (s.c.), as assessed by measuring [³H]DA uptake into purified striatal vesicles prepared from saline- or MPD-treated rats. This increase in vesicular [³H]DA uptake was associated with an increase in binding of the VMAT-2 ligand, [³H]DHTBZ (FIG. 23B). The increases in both vesicular [³H]DA uptake and [³H]DHTBZ binding occur rapidly (i.e., within 30 min) and reversibly (i.e., within 12 h after a 40 mg/kg MPD administration; FIG. 24). At these doses, MPD administration increased locomotor activity and rearing in the treated animals as compared with controls.

The MPD-induced increase in vesicular [³H]DA uptake was associated with an increase in transporter Vₘₐₓ (in fmol/μg protein/3 min: 1584±129 and 2350±250 for saline- and MPD-treated rats, respectively; p≦0.05) with little change in Kₘ (in nM: 235±27 and 230±10 for saline- and MPD-treated rats, respectively; FIG. 25). MPD treatment also increased transporter Bₘₐₓ for the VMAT-2 ligand, [³H]DHTBZ, (in fmol/μg protein: 18.16 and 28.87 for saline- and MPD-treated rats, respectively) with little change in K_{D} (in nM: 3.02 and 3.25 for saline- and MPD-treated rats, respectively). This increase in vesicular [³H]DA uptake did not result from residual MPD introduced by the original in vivo treatment, as direct application of MPD at concentrations of 1 nM to 1 μM was without effect, and higher concentrations of MPD decreased vesicular [³H]DA uptake (i.e., the IC₅₀ for MPD was 19.8±4.0 μM; n=3).

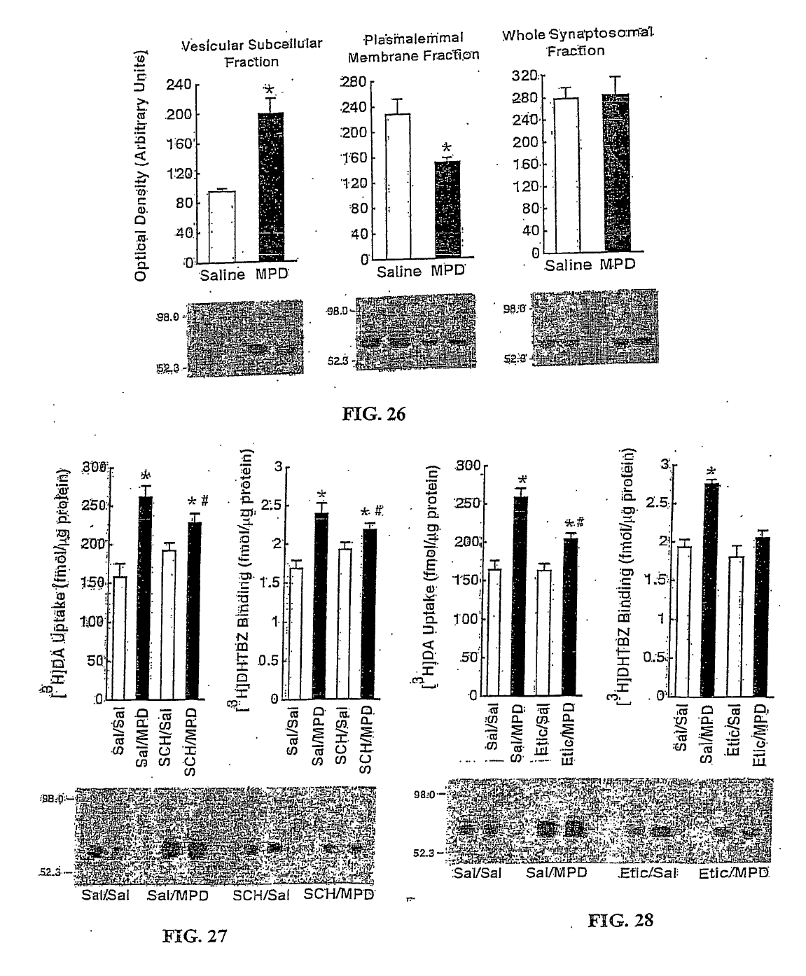

To determine if the MPD-induced increases in vesicular [³H]DA uptake and [³H]DHTBZ binding were associated with an increase in VMAT-2 protein levels, Western blot studies were conducted in three tissue fractions: vesicular subcellular fraction (i.e., synaptic vesicle-enriched), plasmalemmal membrane fraction (i.e., membrane-bound vesicles) and whole synaptosomal fraction (i.e., vesicular subcellular plus plasmalemmal membrane fractions; see Methods for detailed description of fractionation). In accordance with data presented in FIGS. 23 and 24, findings presented in FIG. 26A demonstrate that a single administration of MPD increases VMAT-2 immunoreactivity in the vesicular subcellular fraction. In addition, treatment with MPD decreased VMAT-2 immunoreactivity in the plasmalemmal membrane fraction (FIG. 26B), with no change in the whole synaptosomal fraction (FIG. 26C).

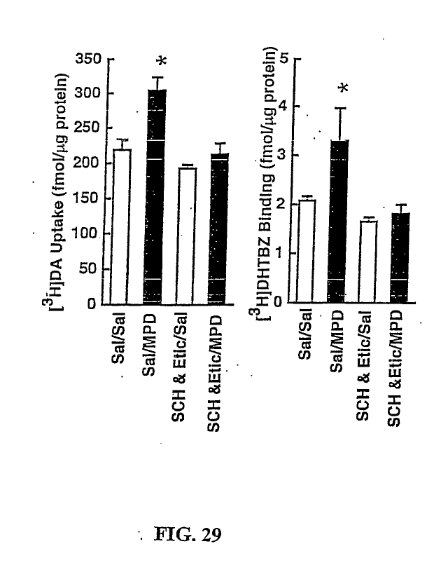

To determine if DA receptor-activation contributed to the MPD-induced increases in vesicular transport, [³H]DHTBZ binding and VMAT-2 protein levels, the DA D₁ receptor antagonist, SCH23390, or the DA D₂ receptor antagonist, eticlopride, was administered prior to MPD treatment. Administration of SCH23390 attenuated the MPD-induced increases in vesicular [³H]DA uptake, [³H]DHTBZ binding and VMAT-2 immunoreactivity in the vesicular subcellular fraction (FIG. 27). Moreover, eticlopride pretreatment attenuated the increase in vesicular [³H]DA uptake and completely prevented the MPD-induced increases in [³H]DHTBZ binding and VMAT-2 immunoreactivity in the vesicular subcellular fraction (FIG. 27). Administration of either SCH23390 or eticloprideper se did not affect vesicular [³H]DA uptake or [³H]DHTBZ binding (FIGS. 27 and 28). Coadministration of these antagonists completely inhibited the increase in vesicular DA sequestration and [³H]DHTBZ binding (FIG. 29).

6. Example Methylphenidate Alters Vesicular Monoamine Transport and Prevents Methamphetamine-Induced Dopaminergic Deficits

a) Materials and Methods

(1) Animals

Male Sprague-Dawley rats (280-340 g; Simonsen Laboratories, Gilroy, Calif.) were maintained under controlled lighting and temperature conditions, with food and water provided ad libitum. All procedures were conducted in accordance with National Institutes of Health Guidelines for the Care and Use of Laboratory Animals and approved by the University of Utah Institutional Animal Care and Use Committee.

(2) Drugs and Chemicals

(±)-MDP hydrochloride and (±)-METH hydrochloride were supplied by the National Institute on Drug Abuse (Bethesda, Md.). 7,8-[³H]DA (42 Ci/mmol) was purchased from Amersham Life Sciences (Arlington Heights, Ill.) and α-[2-³H]DHTBZ (20 Ci/mmol) was purchased from American Radiolabeled Chemicals Inc. (St. Louis, Mo.). Tetrabenazine was kindly donated by Drs. Jeffrey Erickson and Helene Varoqui (Louisiana State University Health Sciences Center, New Orleans, La.). VMAT-2 antibody was purchased from Chemicon International, Inc. (Temecula, Calif.). Doses were calculated as the respective free base and drugs were dissolved in 0.9% saline.

(3) Preparation of Striatal Synaptic Vesicles

Synaptosomes were prepared from rat striatum as described previously (Fleckenstein et al., 1997). Synaptosomes were then resuspended and homogenized in cold distilled deionized water. Osmolarity was restored by addition of N-(2-hydroxyethyl)-piperazine-N′-2-ethanesulfonic acid (HEPES) and potassium tartrate (final concentration in mM: 25 and 100, respectively; pH 7.5). Samples were centrifuged for 20 min at 20,000×g (4° C.) to remove lysed synaptosomal membranes. MgSO₄ (1 mM, final concentration) was added to the supernatant, which was then centrifuged for 45 min at 100,000×g (4° C.). The resulting vesicular pellet was resuspended in wash buffer (see below) at a concentration of 50 mg/ml (original tissue wet weight).

(4) Vesicular [³H]DA Uptake and [³H]DHTBZ Binding

Vesicular [³H]DA uptake was performed by incubating 100 μl (˜2.5 μg protein) of synaptic vesicle samples at 30° C. for 3 min in assay buffer (final concentration in mM: 25 HEPES, 100 potassium tartrate, 1.7 ascorbic acid, 0.05 EGTA, 0.1 EDTA, 2 ATP-Mg²⁺, pH 7.5) in the presence of [³H]DA (30 nM final concentration). The reaction was terminated by addition of 1 ml cold wash buffer (assay buffer containing 2 mM MgSO₄ substituted for the ATP-Mg²⁺, pH 7.5) and rapid filtration through Whatman GF/F filters soaked previously in 0.5% polyethylenimine. Filters were washed three times with cold wash buffer using a Brandel filtering manifold. Radioactivity trapped in filters was counted using a liquid scintillation counter. Nonspecific values were determined by measuring vesicular [³H]DA uptake at 4° C. in wash buffer.

Binding of[³H]DHTBZ was performed as described by Teng et al. (1998). Briefly, 200 μl (˜5 μg protein) of the synaptic vesicle preparation was incubated in wash buffer in the presence of [³H]DHTBZ (2 nM final concentration except in kinetic analyses wherein 0.25-500 nM DHTBZ was employed) for 10 m in at 25° C. The reaction was terminated by addition of 1 ml cold wash buffer and rapid filtration through Whatman GF/F filters soaked in 0.5% polyethylenimine. Filters were washed three times with ice-cold wash buffer. Radioactivity trapped in filters was counted using a liquid scintillation counter. Nonspecific binding was determined by coincubation with 20 μM TBZ. All protein concentrations were determined by a Bio-Rad protein assay (Bio-Rad Inc.).

(5) Preparation of Striatal Subcellular Fractions

Fresh striatal tissue was homogenized in ice-cold 0.32 M sucrose and centrifuged (800×g for 12 min; 4° C.). The resulting supernatant (S1) was then centrifuged (22,000×g for 15 min; 4° C.), and the pellets (P2; whole synaptosomal fraction (plasmalemmal membrane plus vesicular subcellular fractions)) were resuspended in cold distilled deionized water at a concentration of 50 mg/ml (original wet weight of tissue). Resuspended tissue was aliquoted into two test tubes. One aliquot was centrifuged (22,000×g for 20 min; 4° C.) to separate plasmalemmal membranes from the synaptic vesicle-enriched fraction. The resulting supernatant (S3) contained the vesicular subcellular fraction of interest, and the pellets (P3; plasmalemmal membrane fraction) were resuspended in cold distilled deionized water.

(6) Western Blot Analysis

Binding of VMAT-2 antibody was performed using 60 μl of whole synaptosomal, plasmalemmal membrane or vesicle subcellular fractions. Samples were added to 20 μl of loading buffer (final concentration: 2.25% SDS, 18% glycerol, 180 mM Tris Base (pH 6.8), 10% β-mercaptoethanol and bromophenol blue). Approximately 60 μg protein of the whole synaptosomal fraction, 40 μg protein of the plasmalemmal membrane fraction or 20 μg protein of the vesicle subcellular fraction was loaded per well in a 10% SDS-polyacrylamide gel. Following electrophoresis, samples were transferred to polyvinylidene difluoride hybridization transfer membrane (New England Nuclear (NEN), Boston, Mass.). All subsequent incubation steps were performed at room temperature while shaking. Each membrane was first blocked for 2 h in 100 ml of tris buffer saline with tween (TBST; 250 mM NaCl, 50 mM tris pH 7.4 and 0.05% tween 20) containing 5% nonfat dry milk. Each membrane was then incubated with anti-VMAT-2 antibody (1:1000 dilution) in 13 ml of TBST with 5% milk for 1 h and then washed 5 times (2×1 min wash; 3×5 min wash) in 70 ml TBST with 5% milk. The membranes then were incubated for 1 h with the goat F(ab′)₂ anti-rabbit immunoglobulin antibody (Biosource International, Camarillo, Calif.) at a 1:2000 dilution in TBST with 5% milk. This secondary antibody had been affinity-isolated, preabsorbed with human immunoglobulin, and conjugated with horseradish peroxidase. The membranes were then washed 5 times (2×1 min wash; 3×5 min wash) with 70 ml TBST, and then developed with the Renaissance Western Blot Chemiluminescence Reagent Plus (NEN, Boston, Mass.), according to manufacturer specification. Multiple exposures of blots were obtained to ensure development within the linear range of the film (Kodak Biomax MR). Bands on blots were quantified by densitometry measuring net intensity (the sum of the background-subtracted pixel values in the band area) using Kodak 1D image-analysis software.

(7) Vesicular DA Content

Purified striatal vesicles were prepared as described above. The resulting vesicular pellet was sonicated for approximately 5 sec in cold tissue buffer (0.05 M sodium phosphate/0.03 M citric acid buffer with 15% methanol (v/v); pH 2.5) at a concentration of 100 mg/ml (original wet weight of tissue), and centrifuged for 15 min at 22,000×g. Tissue pellets were retained and protein was determined according to the method of Lowry et al. (1951). 40 μl of supernatant was injected onto a high performance liquid chromatograph system coupled to an electrochemical detector (+0.73 V) for separation and quantitation of DA levels using the method of Chapin et al. (1986).

(8) Data Analysis

Statistical analyses among 3 or more groups were performed using an analysis of variance followed by a Fisher PLSD post-hoc comparison. Differences were considered significant if probability of error was less than 5%.

b) Results

Results presented in FIG. 30 demonstrate that multiple administrations of METH (4×7.5 mg/kg; s.c.; 2-h intervals) rapidly decreased VMAT-2 immunoreactivity in a vesicular subcellular fraction (S3), with no change in the whole synaptosomal fraction (P2) or in the plasmalemmal membrane fraction (P3) as assessed in sample prepared 1 h after the final METH injection. In addition, administration of this same METH regimen decreased striatal DA levels with respect to the saline/saline treated group 7 d after treatment (FIG. 31A). Post-treatment with a single MPD injection 1 h after the last METH administration partially reversed the 7-d striatal DA depletions caused by the METH treatment. Two or three injections of MPD (1 and 3 h, or 1, 3 and 5 h, respectively) after the last METH administration completely prevented the persistent METH-induced striatal DA depletions (FIG. 31A). MPD pretreatment per se did not alter total DA levels 7 d after treatment, nor did it prevent the hyperthermia induced acutely by METH administration (FIG. 31B).

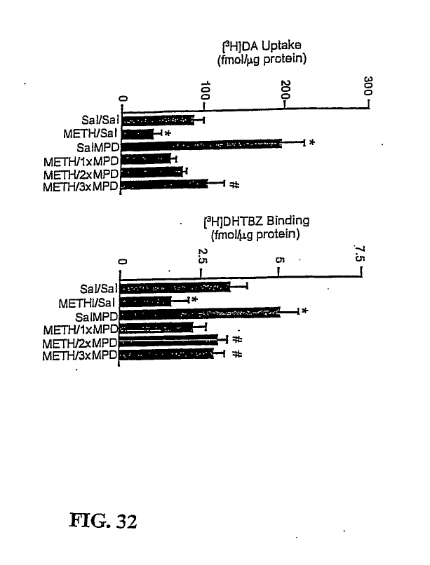

Results presented in FIG. 32 demonstrate that as has been reported previously (Brown et al., 2001), multiple administrations of METH rapidly decreased vesicular DA uptake and DHTBZ binding, as assessed in purified striatal vesicles prepared 1 h after the last METH injection. These effects persisted at least 6 h after treatment (FIG. 32). Post-treating animals with 1, 2 or 3 injections of MPD administered as described for FIG. 31 during the initial 6-h period after METH treatment reversed these rapid METH-induced decreases in vesicular DA uptake and DHTBZ binding (FIG. 33). MPD treatment per se increased vesicular DA uptake and DHTBZ binding (FIG. 33).

Vesicular DA content is a functional consequence of vesicular DA uptake. Accordingly, we investigated the impact of stimulant treatment on vesicular DA content. As a preliminary experiment to validate our assay, rats were treated with reserpine (10 mg/kg, i.p.) 6 and 1 h before decapitation. Predictably, reserpine caused >98% depletion in total striatal tissue DA levels, and striatal vesicular DA levels were below the detection limit of our assay. In another experiment, multiple METH administrations (4×7.5 mg/kg, s.c., 2-h intervals) decreased vesicular DA levels by 49% (i.e., 43.2±6.0 and 2.30±2.5 pg/μg protein for saline and METH-treated rats, respectively; n=6, p≦0.05) as assessed 1 h after treatment. Further results reveal that multiple METH administrations decreased both vesicular and whole tissue DA content by 60%, as assessed 6 h after drug treatment (FIGS. 34A and 34B). In contrast, administration of 3 injections of MPD (administered over a 5 h period as described for FIG. 31) increased vesicular DA levels by ˜140%, without altering total tissue DA concentrations (FIGS. 34A and 34B), as assessed 1 h after the last MPD injection. Finally, post-treatment with 3 injections of MPD immediately after the multiple METH regimen (i.e., both agents administered as described for FIG. 31) reversed (or perhaps compensated for) the METH-induced decrease in vesicular DA content observed 6 h after METH treatment.

### F. REFERENCES

Adams J D Jr, Chang M L, Klaidman L (2001) Parkinson's disease—redox mechanisms. Curr. Med. Chem. 8: 809-814.
Albers D S and Sonsalla P K (1995) Methamphetamine-induced hyperthermia and dopaminergic neurotoxicity in mice: pharmacological profile of protective and nonprotective agents. J Pharmacol Exp Ther 275, 1104-1114.
Alburges M E and Hanson G R (1999) Differential response by neurotensin systems in extrapyramidal and limbic structures to ibogaine and cocaine. Brain Res 181, 96-104.
Alburges M E, Ramos B P, Bush L and Hanson G R (2000) Response of the extrapyramidal and limbic substance P systems to ibogaine and cocaine treatments. Eur J Pharmacol 390, 119-126.
Augustine, G. J., Burns, M. E., DeBello, W. M., Hilfiker, S., Morgan, J. R., Schweizer, F. E., Tokumaru, H., Umayahara, K., 1999. Proteins involved in synaptic vesicle trafficking. J. Physiol. 520, 33-41.
Baker D A, Fuchs R A, Specio S E, Khroyan T V and Neisewander J L (1998) Effects of intraaccumbens of SCH-23390 on cocaine-induced locomotion and conditioned place preference. Synapse 30, 181-193.
Battaglia G, Norman A B, Creese I (1988) Age-related differential recovery rates of rat striatal D-1 dopamine receptors following irreversible inactivation. Eur. J. Pharmacol. 145: 281-290.
Beavo J A, Bechtel P J, Krebs E G (1974) Activation of protein kinase by physiological concentrations of cyclic AMP. Proc. Natl. Acad. Sci. U.S.A. 71: 3580-3583.
Bowyer J, Tank A, Newport G, Slikker W, Ali S, Holson R (1992) The influence of environmental temperature on the transient effects of methamphetamine on dopamine levels and dopamine release in rat striatum. J. Pharmacol. Exp. Ther. 260: 817-824.
Bowyer J F, Davies D L, Schmued L, Broening H W, Newport G D, Slikker W, Holson R R, (1994) Further studies of the role of hyperthermia in methamphetamine neurotoxicity. J Pharmacol Exp Ther 268, 1571-1580.
Brown J M, Hanson G R and Fleckenstein A E (2000) Methamphetamine rapidly decreases vesicular dopamine uptake. J Neurochem 74:2221-2223.

Brown J M, Hanson G R and Fleckenstein A E (2001) Regulation of the vesicular monoamine transporter-2: A Novel mechanism for cocaine and other psychostimulants. J Pharmacol Exp Ther 296:762-767.
Brown J M, Hanson G R, Fleckenstein A E (2001) Cocaine-induced incrases in vesicular dopamine uptake: role of dopamine receptors. J. Pharmacol. Exp. Ther. 298: 1150-1153.
Brown J M, Riddle E L, Sandoval V, Weston R K, Hanson J E, Crosby M J, Ugarte Y V, Gibb J W, Hanson G R, Fleckenstein A E (2002) A single methamphetamine administration rapidly decreases vesicular dopamine uptake. J Pharmacol Exp Ther 302: 497-501.
Buening M K and Gibb J W (1974) Influence of methamphetamine and neuroleptic drugs on tyrosine hydroxylase activity. Eur J Pharmacol 26: 30-34.
Butcher S P, Liptrot J, Aburthnott G W (1991) Characterisation of methylphenidate and nomifensine induced dopamine release in rat striatum using in vivo brain microdialysis. Neurosci. Lett. 122: 245-248.
Challman T D, Lipsky J J (2000) Methylphenidate: its pharmacology and uses. Mayo Clin. Proc. 75: 711-721.
Chapin, D. S., Lookingland, K. J., Moore, K. E., 1986. Effects of LC mobile phase composition on retention times for biogenic amines, and their precursors and metabolites. Curr. Separations 7:68-70
Cohen G (1990) Monoamine oxidase and oxidative stress at dopaminergic synapses. J. Neural. Transm. Suppl. 32: 229-238.
Cubells, J. F., Rayport, S., Rajendran, G., Sulzer, D., 1994. Methamphetamine neurotoxicity involves vacuolation of endocytic organelles and dopamine-dependent intracellular oxidative stress. J. Neuorsci. 14, 2260-2271.
Eisch A J, Gaffney M, Weibmuller F B, O'Dell S J and Marshall J F (1992) Striatal subregions are differentially vulnerable to the neurotoxic effects of methamphetamine. Brain Res 598:321-326.
Erickson J D, Eiden L E, Hoffman B J (1992) Expression cloning of a reserpine-sensitive vesicular monoamine transporter. Proc. Natl. Acad. Sci. U.S.A. 89:10993-10997.
Fahn S, Cohen G (1992) The oxidant stress hypothesis in Parkinson's disease: evidence supporting it. Ann. Neurol. 32: 804-812.
Falkenburger B H, Barstow K L, Mintz I M (2001) Dendrodendritic inhibition through reversal of dopamine transport. Science 293: 2465.
Fleckenstein A E, Gibb J W and Hanson G R (2000) Differential effects of stimulants on monoaminergic transporters: pharmacological consequences and implications for neurotoxicity. Eur J Pharmacol. 406:1-13.
Fleckenstein A E, Haughey H M, Metzger R R, Kokoshka J M, Riddle E L, Hanson J E and Hanson G R (1999) Differential effects of psychostimulants and related agents on dopaminergic and serotonergic transporter function, Eur J Pharmacol 382:45-49.

Fleckenstein A B, Metzger R R, Wilkins D G, Gibb J W and Hanson G R (1997) Rapid and reversible effects of methamphetamine on dopamine transporters. J Pharmacol Exp Ther 282:834-838
Fleckenstein A E, Pogun S, Carroll F I, Kuhar M J (1996) Recovery of dopamine transporter binding and function after intrastriatal administration of the irreversible inhibitor RTI-76 [3-beta-(3p-chlorophenyl)tropan-2 beta-carboxylic acid p isothiocyanatophenylethyl ester hydrochloride]. J. Pharmacol. Exp. Ther. 279: 200-206.
Fleckenstein A E, Wilkins D G, Gibb J W, Hanson G R (1997) Interaction between hyperthermia in the 5-hydroxytryptaminergic response to a single methamphetamine administration. J Pharmacol Exp Ther 283: 281-285.
Frey, K., Kilbourn, M., Robinson, T., 1997. Reduced striatal vesicular monoamine transporters after neurotoxic but not after behabiorally-sensitizing doses of methamphetamine. Eur. J. Pharmacol. 334, 273-279.
Fuller R W, Hemrick-Luecke S (1980) Long-lasting depletion of striatal dopamine by a single injection of amphetamine in iprindole-treated rats. Science 209: 305-307.
Fuller R W, Hemrick-Luecke S K (1982) Further studies on the long-term depletion of striatal dopamine in iprindole-treated rats by amphetamine. Neuropharmacology. 21:433-438.
Fumagalli F, Gainetdinov R R, Wang Y M, Valenzano K J, Miller G W, Caron M G (1999) Increased methamphetamine neurotoxicity in heterozygous vesicular monoamine transporter 2 knock-out mice. J Neurosci. 19: 2424-2431.
Gainetdinov R R, Fumagalli F, Wang Y M, Jones S R, Levey A I, Miller G W, Caron M G (1998) Increased MPTP neurotoxicity in vesicular monoamine transporter 2 heterozygote knockout mice. J. Neurochem. 70: 1973-1978.
German D C, Liang C L, Manaye K F, Lane K, Sonsalla P K (2000) Pharmacological inactivation of the vesicular monoamine transporter can enhance 1-methyl-4-phenyl-1,2,3,6,tetrahydropyridine-induced neurodegeneration of midbrain dopaminergic neurons, but not locus coeruleus noradrenergic neurons. Neuroscience 101: 1063-1069.
Graham D G, Tiffany S M, Bell W R Jr, Gutknecht W F (1978) Autoxidation versus covalent binding of quinones as the mechanism of toxicity of dopamine, 6-hydroxydopamine, and related compounds toward C1300 neuroblastoma cells in vitro. Mol Pharmacol 14: 644-653.
Hansen J P, Riddle E L, Sandoval V, Brown J M, Gibb J W, Hanson G R, Fleckenstein A E. (2002) Methylenedioxymethamphetamine decreases plasmalemmal and vesicular dopamine transport: mechanisms and implications for neurotoxicity. J. Pharmacol. Exp. Ther. 300:1093-1100.
Hastings T G (1995) Enzymatic oxidation of dopamine: the role of prostaglandin H synthase. J Neurochem 64: 919-924.
Haughey H M, Fleckenstein A E and Hanson G R (1999) Differential regional effects of methamphetamine on the activities of tryptophan and tyrosine hydroxylase. J Neurochem 72:661-668.
Hogan K A, Staal R G, Sonsalla P K (2000) Analysis of VMAT2 binding after methamphetamine or MPTP treatment: disparity between homogenates and vesicle preparations. J. Neurochem. 74: 2217-2220.
Hotchkiss A J, Morgan M E and Gibb J W (1979) The long-term effects of multiple doses of methamphetamine on neostriatal tryptophan hydroxylase, tryosine hydroxylase, choline acetyltransferase and glutamate decarboxylase activities. Life Sci 25:1373-1378.
Hurd Y L, Ungerstedt U (1989) In vivo neurochemical profile of dopamine uptake inhibitors and releasers in rat caudate-putamen. Eur. J. Pharmacol. 166: 251-260.
Insel T R, Battaglia G, Johannessen J N, Marra S and De Souza E B (1989) 3,4-Methylenedioxymethamphetamine (“ecstasy”) selectively destroys brain serotonin terminals in rhesus monkeys. J Pharmacol Exp Ther 249:713-720.
Iwata, S., Hewlett, G. H., Ferrell, S. T., Czemik, A. J., Meiri, K. F., Gnegy, M. E., 1996. Increased in vivo phosphorylation state of neuromodulin and synapsin I in striatum from rats treated with repeated amphetamine. J. Pharmacol. Exp. Ther. 278, 1428-1434.
Iwata, S., Hewlett, G. H., Gnegy, M. E., 1997. Amphetamine increases the phosphorylation of neuromodulin and synapsin I in rat striatal synaptosomes. Synapse. 26, 281-291.
Izenwasser S, Coy A E, Ladenheim B, Loeloff R J, Cadet J L, French D (1999) Chronic methylphenidate alters locomotor activity and dopamine transporters differently from cocaine. Eur. J. Pharmacol. 373: 187-193.
Jenner P (1998) Oxidative mechanisms in nigral cell death in Parkinson's disease. Mov. Disord. 13(Suppl. 1): 24-34.
Johnson M, Letter A A, Merchant K, Hanson G R and Gibb J W (1988) Effects of 3,4-methylenedioxyamphetamine and 3,4-methylenedioxymethamphetamine isomers on central serotonergic, dopaminergic and nigral neurotensin systems of the rat. J Pharmacol Exp Ther 244:977-982.
Johnston L D, O'Malley P M and Bachman J G (2000) “Ecstasy” use rises sharply among teens in 2000; use of many other drugs steady, but significant declines are reported for some. University of Michigan News and Information Services: Ann Arbor, Mich. [On-line]. Available: www.monitoringthefuture.org; accessed Jun. 8, 2001.
Kadota K and Kadota T (1973) Isolation of coated vesicles, plain synaptic vesicles, and flocculent material from a crude synaptosome fraction of guinea pig whole brain. J Cell Biol 58:135-151.
Koda L Y and Gibb J W (1973) Adrenal and striatal tyrosine hydroxylase activity after methamphetamine. J Pharmacol Exp Ther 185: 42-48.
Kokoshka J M, Vaughan R A, Hanson G R and Fleckenstein A E (1998) Nature of methamphetamine-induced rapid and reversible changes in dopamine transporters. Eur J Pharmacol 361:269-275.
LaVoie M J and Hastings T G (1999) Dopamine quinone formation and protein modification associated with the striatal neurotoxicity of methamphetamine: evidence against a role for extracellular dopamine. J. Neurosci 19(4):1484-91
LaVoie, M. J., Hastings, T. G., 1999. Peroxynitrite- and nitrite-induced oxidation of dopamine: implications for nitric oxide in dopaminergic cell loss. J. Neurochem. 73, 2546-2554.
Liang N Y, Rutledge C. O. (1982) Comparison of the release of [³H]dopamine from isolated corpus striatum by amphetamine, fenfluramine and unlabelled dopamine. Biochem. Pharmacol. 31: 983-992.
Liu Y, Edwards R H (1997) The role of vesicular transport proteins in synaptic transmission and neural degeneration. Annu. Rev. Neurosci. 20: 125-156.
Liu Y, Peter D, Roghani A, Schuldiner S, Prive G G, Eisenberg D, Brecha N, Edwards R H (1992) A cDNA that suppresses MPP+ toxicity encodes a vesicular amine transporter. Cell 70(4): 539-551.
Liu Y, Roghani A, Edwards R H (1992) Gene transfer of a reserpine-sensitive mechanism of resistance to N-methyl-4-phenylpyridinium. Proc Natl Acad Sci USA. 89: 9074-9078.
Lowry O H, Rosebrough N J, Farr A L and Randall R J (1951) Protein measurement with the folin phenol reagent. J Biol Chem 193:265-275.
Mack F, Bonisch H (1979) Dissociation constants and lipophilicity of catecholamines and related compounds. Naunyn Schmiedebergs Arch. Pharmacol. 310:1-9.
Maker H S, Weiss C, Silides D J, Cohen G (1981) Coupling of dopamine oxidation (monoamine oxidase activity) to glutathione oxidation via the generation of hydrogen peroxide in rat brain homogenates. J Neurochem 36:589-593.
Marek G J, Vosmer G, Seiden L S (1990) Dopamine uptake inhibitors block long-term neurotoxic effects of mehtamphetamine upon dopaminergic neurons. Brain Res. 513: 274-279.
Metzger R R, Hanson G R, Gibb J W and Fleckenstein A E (1998) 3-4-Methylenedioxymethamphetamine-induced acute changes in dopamine transporter function. Eur J Pharmacol 349:205-210.
Metzger R R, Haughey H M, Wilkins D G, Gibb J W, Hanson G R and Fleckenstein A E (2000) Methamphetamine-induced rapid decrease in dopamine transporter function: role of dopamine and hyperthermia. J Pharmacol Exp Ther 295:1077-1085.
Miller G W, Gainetdinov R R, Levey A I, Caron M G (1999) Dopamine transporters and neuronal injury. Trends Pharmacol Sci 20: 424-429.
Morgan M E and Gibb J W (1980) Short-term and long-term effects of methamphetamine on biogenic amine metabolism in extra-striatal dopaminergic nuclei. Neuropharmacology 19:989-995
Nirenberg M J, Chan J. Liu Y, Edwards R G and Pickel V M (1997) Vesicular Monoamine Transporter-2: immunogold localization in striatal axons and terminals. Synapse 26:194-196.
Norman A B, Battaglia G, Creese I (1987) Differential recovery rates of rat D₂ dopamine receptors as a function of aging and chronic reserpine treatment following irreversible modification: a key to receptor regulatory mechanisms. J. Neurosci. 7: 1484-1491.
Pan D, Gatley S J, Dewey S L, Chen R, Alexoff D A, Ding Y, Fowler J S (1994) Binding of bromine-substituted analogs of methylphenidate to monoamine transporters. Eur. J. Pharmacol. 264: 177-182.
Pincus H A, Wise T, First M B (1995) Diagnostic and Statistical Manual of Mental Disorders, Primary Care Version, Fourth edition, pp. 182-184. Washington, D.C.: American Psychiatric Association.
Reinhard J F, Jr, Deliberto E J, Humberto Viveros O, Daniels A J (1987) Subcellular compartmentilization of 1-methyl-4-phenylpyridinium with catecholamines in adrenal chromaffinn vesicles may explain the lack of toxicity to adrenal chromaffin cells. Proc. Natl. Acad. Sci. U.S.A. 84: 8160-8164.
retention times for biogenic amines, and their precursors and metabolites. Curr. Sep 7: 68-70.
Riddle E L, Tophan M K, Haycock J W, Hanson G R, Fleckenstein A E (2002) Differential trafficking of the vesicular monoamine transporter-2 by methamphetamine and cocaine. Eur J Pharmacol 449:71-74.
Ritz M C, Lamb R J, Goldberg S R, Kuhar M J (1987) Cocaine receptors on dopamine transporters are related to self-administration of cocaine. Science 237: 1219-1223.
Sandoval V, Riddle E L, Ugarte Y V, Hanson G R and Fleckenstein A E (2001) Methamphetamine-induced rapid and reversible changes in dopamine transporter function: an in vitro model. J Neurosci 21:1413-1419.
Saunders C, Ferrer J V, Shi L, Chen J, Merrill G, Lamb M E, Leeb-Lundberg L M F, Carvelli L, Javitch J A and Galli A (2000) Amphetamine-induced loss of human dopamine transporter activity: an internalization-dependent and cocaine-sensitive mechanism. Proc Natl Acad Sci USA 97:6850-6855.
Schmidt C J, Gibb J W (1985a) Role of the dopamine uptake carrier in the neurochemical response to methamphetamine: effects of amfonelic acid. Eur. J. Pharmacol. 109: 73-80.
Schmidt C J, Ritter J K, Sonsalla P K, Hanson G R, Gibb J W (1985b) Role of dopamine in the neurotoxic effects of methamphetamine. J. Pharmacol. Exp. Ther. 233: 539-544.
Schmitz Y, Lee C J, Schmauss C, Gonon F, Sulzer D (2001) Amphetamine distorts stimulation-dependent dopamine overflow: effects on D2 autoreceptors, transporters, and synapticvesicle stores. J Neurosci 15, 5916-5924
Seiden L S, Fischman M W and Schuster C R (1976) Long-term methamphetamine induced changes in brain catecholamines in tolerant rhesus monkeys. Drug Alcohol Depend 1:215-219.
Speciale S G, Liang C L, Sonsalla P K, Edwards R H, German D. C. (1998) The neurotoxin 1-methyl-4-phenylpyridinium is sequestered within neurons that contain the vesicular monoamine transporter. Neuroscience 84: 1177-1185.
Staal R G, Sonsalla P K (2000) Inhibition of brain vesicular monoamine transporter (VMAT-2) enhances 1-methyl-4-phenylpyridinium neurotoxicity in vivo in rat striata. J. Pharmacol. Exp. Ther. 293: 336-342.
Stoof J C, Kebabian J W (1981) Opposing roles for D-1 and D-2 dopamine receptors in efflux of cyclic AMP from rat neostriatum. Nature 294: 366-368.
Sulzer D, Chen T K, Lau Y Y, Kristensen H, Rayport S, Ewing A (1995) Amphetamine redistributes dopamine from synaptic vesicles to the cytosol and promotes reverse transport. J Neurosci 15, 4102-4108.
Sulzer D, Rayport S (1990) Amphetamine and other psychostimulants reduce pH gradients in midbrain dopaminergic neurons and chromaffin granules: a mechanism of action. Neuron 5(6): 797-808.
Sulzer, D., Chen, T. K., Lau, Y. Y., Kristensen, H., Rayport, S., Ewing A., 1995. Amphetamine redistributes dopamine from synaptic vesicles to the cytosol and promotes reverse transport. J. Neurosci. 5, 4102-4108.
Takahashi N, Mine L L, Sora I, Ujike H, Revay R S, Kostic V, Jackson-Lewis V, Predborski S, Uhl G R (1997) VMAT2 knockout mice: heterozygotes display reduced amphetamine-conditioned reward, enhanced amphetamine locomotion, and enhanced MPTP toxicity. Proc Natl Acad Sci USA. 94: 9938-9943.
Teng L, Crooks P A, Dwoskin L P (1998) Lobeline displaces [³H]dihydrotetrabenazine binding and releases [³H]dopamine from rat striatal synaptic vesicles: comparison with d-amphetamine. J. Neurochem. 71: 258-265.
Teng L, Crooks P A, Sonsalla P K and Dwoskin L P (1997) Lobeline and nicotine evoke [³H]dopamine overflow from rat striatal slices preloaded with dopamine: differential inhibition of synaptosomal and vesicular [³H]dopamine uptake. J Pharmacol Exp Ther 280:1432-1444.
U.S. Department of Justice, Drug Enforcement Administration, Diversion Enforcement Administration: Stimulant abuse by school age children: a guide for school officials (2001) htpp://www.deadiversion.usdoj.gov:80/pubs/brochures/stimulant/stimulant_abuse.htm #f.
Vallar L, Meldolesi J (1989) Mechanisms of signal transduction at the dopamine D₂ receptor. Trends Pharmacol. Sci. 10: 74-77.
Villemagne V, Yuan J, Wong D F, Dannals R F, Hatzidimitriou G, Mathews W B, Ravert H T, Musachio J, McCann U D, Ricaurte G A (1998) Brain dopamine neurotoxicity in baboons treated with doses of methamphetamine comparable to those recreationally abused by humans: evidence from [11C]WIN-35,428 positron emission tomography studies and direct in vitro determinations. J Neurosci 18:419-27
Wagner G C, Lucot J B, Schuster C R, Seiden L S (1983) Alpha-methyltyrosine attenuates and reserpine increases methamphetamine-induced neuronal changes. Brain Res 270: 285-288.
Wang G J, Volkow N D, Fowler J S, Fischman M, Foltin R, Abumrad N N, Logan J, Pappas N R (1997) Cocaine abusers do not show loss of dopamine transporters with age. Life Sci 61:1059-1065.
Wieczorek W J, Kruk Z L (1994) Differential action of (+)-amphetamine on electrically evoked dopamine overflow in rat brain slices containing corpus striatum and nucleus accumbens. Br J Pharmacol 111, 829-836
Wilson, J. M., Kalasinsky, K. S., Levey, A. I., Bergeron, C., Reiber, G., Anthony, R. M., Schmunk, G. A., Shannak, K., Haycock, J. W., Kish, S. J., 1996a. Striatal dopamine nerve terminal markers in human, chronic methamphetamine users. Nat. Med. 2, 699-703.
Wilson, J. M., Kish, S. J., 1996. The vesicular monoamine transporter, in contrast to the dopamine transporter, is not altered by chronic cocaine self-administration in the rat. J. Neurosci. 16, 3507-3510.
Wilson, J. M., Levey, A. I., Bergeron, C., Kalasinsky, K., Ang, L., Peretti, F., Adams, V. I., Smialek, J., Anderson, W. R., Shannak, K., Deck, J., Niznik, H. B., Kish, S. J., 1996b. Striatal dopamine, dopamine transporter, and vesicular monoamine transporter in chronic cocaine users. Ann Neurol. 40, 428-439
Zaczek R, Culp S, De Souza E. B (1991a) Interactions of [³H]amphetamine with rat brain synaptosomes. II. Active transport. J. Pharmacol. Exp. Ther. 257: 830-835.
Zaczek R, Culp S, Goldberg H, McCann D J, De Souza E B (1991b) Interactions of [³H]amphetamine with rat brain synaptosomes. I. Saturable sequestration. J. Pharmacol. Exp. Ther. 257: 820-829.
Zuddas A, Ancilletta B, Muglia P, Cianchetti C (2000) Attention-deficit/hyperactivity disorder: a neuropsychiatric disorder with childhood onset. Eur. J. Paediatr. Neurol. 4: 53-62.

### G. SEQUENCES

1. VMAT Sequences Incorporated by Reference
a) NP_{—}037163 Links solute carrier family 18 A2 (vesicular monoamine transporter 2) [Rattus norvegicus] gi|6981546|re|NP_{—}037163.1|[6981546]
b) 2: NP_{—}006623 Links solute carrier family 17 (sodium phosphate), member 3 [Homo sapiens] gi|5730047|ref|NP_{—}006623.1|[5730047]
c) 3: NP_{—}003046 Links solute carrier family 18 (vesicular acetylcholine), member 3 [Homo sapiens] gi|4506991|ref|NP_{—}003046.1|[4506991]
d) 4: NP_{—}003045 Links solute carrier family 18 (vesicular monoamine), member 2 [Homo sapiens] gi|4506989|ref|NP_{—}003045.1|[4506989]
e) 5: NP_{—}003044 Links solute carrier family 18 (vesicular monoamine), member 1 [Homo sapiens] gi|4506987|ref|NP_{—}003044.1|[4506987]

2. VMAT Sequences

a) NP_{—}037163. LOCUS Slc18a2 515 aa linear ROD 1 Nov. 2000 DEFINITION solute carrier family 18 A2 (vesicular monoamine transporter 2) [Rattus norvegicus].

| | |
|---|---|
| 1 | malsdlvllr wlrdsrhsrk lilfivflal lldnmlltvv vpiipsylys ikheknstei |
| 61 | qttrpelvvs tsesifsyyn nstvlitgna tgtlpggqsh katstqhtva nttvpsdcps |
| 121 | edrdllnenv qvgllfaska tvqlltnpfi glltnrigyp ipmfagfcim fistvmfafs |
| 181 | ssyaflliar slqgigsscs svagmgmlas vytddeergn amgialggla mgvlvgppfg |
| 241 | svlyefvgkt apflvlaalv lldgaiqlfv lqpsrvqpes qkgtplttll kdpyiliaag |
| 301 | sicfanmgia mlepalpiwm metmcsrkwq lgvaflpasi syligtnifg ilabkmgrwl |
| 361 | callgmvivg isilcipfak niygliapnf gvgfaigmvd ssmmpimgyl vdlrhvsvyg |
| 421 | svyaiadvaf cmgyaigpsa ggaiakaigf pwlmtiigii diafapicff lrsppakeek |
| 481 | mailmdhncp iktkmytqnn vqsypigdde esesd |

b) cDNA for NP_{—}037163.

| | |
|---|---|
| 1 | atggccctga gcgatctggt gctgctgcga tggctgcggg acagccgcca ctcgcgcaaa |
| 61 | ctgatcctgt tcatcgtgtt ccttgcgctg ctgctggaca acatgctgct caccgtcgtg |
| 121 | gttcccatca tccccagcta tctgtacagc attaagcatg agaaaaactc tacggaaatc |
| 181 | cagaccacca gaccagagct cgtggtctcc acctccgaaa gcatcttctc ttactataac |
| 241 | aactctactg tgttgateac cgggaatgcc actgggactc ttccaggagg gcagtcacac |
| 301 | aaggctacca gcacacagca cactgtggct aacaccactg tcccttcgga ctgtcccagt |
| 361 | gaagacagag accttctgaa tgagaatgtg caagttgggc tgctgtttgc ctccaaagcc |
| 421 | actgtccagc tcctcactaa cccattcata ggacttctga ccaacagaat tggctatcca |
| 481 | attcccatgt ttgccggctt ctgcatcatg tttatctcaa cagttatgtt tgccttctcc |
| 541 | agcagctatg ccttcctgct gatcgccagg tcccttcagg gaattggctc ctcctgctca |
| 601 | tccgtggctg ggatgggtat gctggccagc gtgtacacag atgatgagga gagggggaac |
| 661 | gccatgggca ttgctttggg tggcctggcc atgggagtct tagtgggacc ccccttcggg |
| 721 | agtgtgctct atgagtttgt ggggaagaca gctcccttcc tggtgctagc tgccttggtg |
| 781 | ctcttggatg gggctattca gctctttgtg ctccagccgt cccgagtaca gccagagagt |
| 841 | cagaagggga cacctctaac gaccttgctg aaggatccat acatcctcat cgctgcaggc |
| 901 | tccatctgct ttgcaaacat ggggatagcc atgctggagc ccgccctgcc catctggatg |
| 961 | atggagacca tgtgttcccg aaagtggcag ctgggcgttg ctttcctccc ggcgagcatc |
| 1021 | tcttatctca ttggaaccaa tatttttggg atacttgcac acaaaatggg aaggtggcta |
| 1081 | tgtgctcttc tgggaatggt aattgttgga atcagcattt tatgcatccc ctttgcaaaa |
| 1141 | aatatctatg gaetcatcgc tcccaacttt ggagttggtt ttgcaattgg gatggtggac |
| 1201 | tcctctatga tgcctatcat gggctacctg gttgacctgc ggcatgtgtc tgtctatggg |
| 1261 | agtgtttatg ccattgcaga cgtggccttt tgtatgggct atgctatcgg tccctctgct |
| 1321 | ggtggtgcca tcgcaaaggc aattggcttt ccttggctta tgacaattat tgggataatt |
| 1381 | gatatcgctt ttgctccact ctgctttttc cttcgaagtc cacctgctaa ggaggaaaaa |
| 1441 | atggctatcc tcatggacca caactgtccc attaaaacaa agatgtacac tcagaataat |
| 1501 | gtccagtcat atcccatcgg tgatgatgaa gaatctgaaa gtgactga |

c) NP_{—}003045. solute carrier fa . . . [gi:4506989] Links LOCUS SLC18A2 514 aa linear PRI 31 Oct. 2000 DEFINITION solute carrier family 18 (vesicular monoamine), member 2 [Homo sapiens].

| | |
|---|---|
| 1 | malselalvr wlqesrhsrk lilfivflal lldnmlltvv vpiipsylys ikheknatei |
| 61 | qtarpvhtas isdsfqsifs yydnstmvtg natrdltlhq tatqhmvtna savpsdcpse |
| 121 | dkdllnenvq vgllfaskat vqlitnpfig lltnrigypi pifagfcimf vstmifafss |
| 181 | syaflliars lqgigsscss vagmgmlasv ytddeergnv mgialgglam gvlvgppfgs |
| 241 | vlyefvgkta pflvlaalvl ldgaiqlfvl qpsrvqpesq kgtplttllk dpyiliaags |
| 301 | icfanmgiam lepalpiwmm etmcsrkwql gvaflpasis yligtnifgi lahkmgrwlc |
| 361 | allgmiivgv silcipfakn iygliapnfg vgfaigmvds smmpimgylv dlrhvsvygs |
| 421 | vyaiadvafc mgyaigpsag gaiakaigfi wlmtiigiid ilfaplcffi rsppakeekm |
| 481 | ailmdhncpi ktkmytqnni qsypigedee sesd |

d) cDNA for NP_{—}003045

| | |
|---|---|
| 1 | atggccctga gcgagctggc gctggtccgc tggctgcagg agagecgcca ctcgcggaag |
| 61 | ctcatcctgt tcatcgtgtt cctggcgctg ctgctggaca acatgctgct cactgtcgtg |
| 121 | gtccccatca tcccaagtta tctgtacagc attaagcatg agaagaatgc tacagaaatc |
| 181 | cagacggcca ggccagtgca cactgcctcc atctcagaca gcttccagag catcttctcc |
| 241 | tattatgata actcgactat ggtcaccggg aatgctacca gagacctgac acttcatcag |
| 301 | accgccacac agcacatggt gaccaacgcg tccgctgttc cttccgactg tcccagtgaa |
| 361 | gacaaagacc tcctgaatga aaacgtgcaa gttggtctgt tgtttgcctc gaaagccacc |
| 421 | gtccagctca tcaccaaccc tttcatagga ctactgacca acagaattgg ctatccaatt |
| 481 | cccatatttg cgggattctg catcatgttt gtctcaacaa ttatgtttgc cttctccagc |
| 541 | agctatgcct tcctgctgat tgccaggtcg ctgcagggca tcggctcgtc ctgctcctct |
| 601 | gtggctggga tgggcatgct tgccagtgtc tacacagatg atgaagagag aggcaacgtc |
| 661 | atgggaatcg ccttgggagg cctggccatg ggggtcttag tgggcccccc cttcgggagt |
| 721 | gtgctctatg agtttgtggg gaagacggct ccgttcctgg tgctggccgc cctggtactc |
| 781 | ttggatggag ctattcagct ctttgtgctc cagccgtccc gggtgcagcc agagagtcag |
| 841 | aaggggacac ccctaaccac gctgctgaag gacccgtaca tcctcattgc tgcaggctcc |
| 901 | atctgctttg caaacatggg catcgccatg ctggagccag ccctgcccat ctggatgatg |
| 961 | gagaccatgt gttcccgaaa gtggcagctg ggcgttgcct tcttgccagc tagtatctct |
| 1021 | tatctcattg gaaceaatat ttttgggata cttgcacaca aaatggggag gtggctttgt |
| 1081 | gctcttctgg gaatgataat tgttggagtc agcattttat gtattccatt tgcaaaaaac |
| 1141 | atttatggac tcatagctcc gaactttgga gttggttttg caattggaat ggtggattcg |
| 1201 | tcaatgatgc ctatcatggg ctacctcgta gacctgcggc acgtgtccgt ctatgggagt |
| 1261 | gtgtacgcca ttgcggatgt ggcattttgt atggggtatg ctataggtcc ttctgctggt |
| 1321 | ggtgctattg caaaggcaat tggatttcca tggctcatga caattattgg gataattgat |
| 1381 | attctttttg cccctctctg cttttttctt cgaagtccac ctgccaaaga agaaaaaatg |
| 1441 | gctattctca tggatcacaa ctgccctatt aaaacaaaaa tgtacactca gaataatatc |
| 1501 | cagtcatatc cgataggtga agatgaagaa tctgaaagtg actga |

e) 3: NP_{—}003044. solute carrier fa . . . [gi:4506987] Links LOCUS SLC18A1 525 aa linear PRI 31 Oct. 2000 DEFINITION solute carrier family 18 (vesicular monoamine), member 1 [Homo sapiens].

| | |
|---|---|
| 1 | mlrtildapq rllkegrasr qlvlvvvfva llldnmlftv vvpivptfly dmefkevnss |
| 61 | lhlghagssp halaspafst ifsffnnntv aveesvpsgi awmndtasti pppateaisa |
| 121 | hknnclqgtg fleeeitrvg vlfaskavmq llvnpfvgpl tnrigybipm fagfvimfls |
| 181 | tvmfafsgty tllfvartlq gigssfssva glgmlasvyt ddhergramg taigglalgi |
| 241 | lvgapfgsvm yefvgksapf lilaflalld galqlcilqp skvspesakg tplfmhlkdp |
| 301 | yilvaagsic fanmgvaile ptlpiwmmqt mcspkwqlgl aflpasvsyl igtnlfgvla |
| 361 | nkmgrwlcsl igmlvvstsl lcvplahnif gligpnaglg laigmvdssm mpimghlvdl |
| 421 | rhtsvygsvy aiadvafcmg faigpstgga ivkaigfpwl mvitgviniv yaplcyylrs |
| 481 | ppakeeklai lsqdcpmetr myatqkptke fplgedsdee pdhee |

f) cDNA for NP_{—}003044

| | |
|---|---|
| 1 | atgctccgga ccattctgga tgctccccag cggttgctga aggaggggag agcgtcccgg |
| 61 | cagctggtgc tggtggtggt attcgtcgct ttgctcctgg acaacatgct gtttactgtg |
| 121 | gtggtgccaa ttgtgcccac cttcctatat gacatggagt tcaaagaagt caactcttct |
| 181 | ctgcacctcg gccatgccgg aagttcccca catgccctcg cctctcctgc cttttccacc |
| 241 | atcttctcct tcttcaacaa caacaccgtg gctgttgaag aaagcgtacc tagtggaata |
| 301 | gcatggatga atgacactgc cagcaccatc ccacctccag ccactgaagc catctcagct |
| 361 | cataaaaaca actgcttgca aggcacaggt ttcttggagg aagagattac ccgggtcggg |
| 421 | gttctgtttg cttcaaaggc tgtgatgcaa cttctggtca acccattcgt gggecctctc |
| 481 | accaacagga ttggatatca tatccccatg tttgctggct ttgttatcat gtttctctcc |
| 541 | acagttatgt ttgctttttc tgggacctat actctactct ttgtggcccg aacccttcaa |
| 601 | ggcattggat cttcattttc atctgttgca ggtcttggac tgctggccag tgtctacact |
| 661 | gatgaccatg agagaggacg agccatggga actgctctgg ggggcctggc cttggggttg |
| 721 | ctggtgggag ctccctttgg aagtgtaatg tacgagtttg ttgggaagtc tgcacccttc |
| 781 | ctcatcctgg ccttcctggc actactggat ggagcactcc agctttgcat cctacagcct |
| 841 | tccaaagtct ctcctgagag tgccaagggg actcccctct ttatgcttct caaagaccct |
| 901 | tacatcctgg tggctgcagg gtccatctgc tttgccaaca tgggggtggc catcctggag |
| 961 | cccacactgc ccatctggat gatgcagacc atgtgctccc ccaagtggca gctgggtcta |
| 1021 | gctttcttgc ctgccagtgt gtcctacctc attggcacca acctctttgg tgtgttggcc |
| 1081 | aacaagatgg gtcggtggct gtgttcccta atcgggatgc tggtagtagg taccagettg |
| 1141 | ctctgtgttc ctctggctca caatattttt ggtctcattg gccccaatgc agggcttggc |
| 1201 | cttgccatag gcatggtgga ttcttctatg atgcccatca tggggcacct ggtggatcta |
| 1261 | cgccacacct cggtgtatgg gagtgtctac gccatcgctg atgtggcttt ttgcatgggc |
| 1321 | tttgctatag gtccatccac cggtggtgcc attgtaaagg ccatcggttt tccctggctc |
| 1381 | atggtcatca ctggggtcat caacatcgtc tatgctccac tctgctacta cctgcggagc |
| 1441 | cccccggcaa aggaagagaa gcttgctatt ctgagtcagg actgccccat ggagacccgg |
| 1501 | atgtatgcaa cccagaagcc cacgaaggaa tttcctctgg gggaggacag tgatgaggag |
| 1561 | cctgaccatg aggatag |

3. Dopamine D1 Receptor

a) NP_{—}000785. dopamine receptor . . . [gi:4503383] Links LOCUS DRD1 446 aa linear PRI 27 Aug. 2002 DEFINITION dopamine receptor D1 [Homo sapiens].

| | |
|---|---|
| 1 | mrtlntsamd gtglvverdf svriltacfl sllilstllg ntlvcaavir frhlrskvtn |
| 61 | ffvislavsd llvavlvmpw kavaeiagfw pfgsfcniwv afdimcstas ilnlcvisvd |
| 121 | rywaisspfr yerkmtpkaa filisvawtl svlisfipvq lswhkakpts psdgnatsla |
| 181 | etidncdssl srtyaisssv isfyipvaim ivtytriyri aqkqirriaa leraavhakn |
| 241 | cqtttgngkp vecsqpessf kmsfkretkv lktlsvimgv fvccwlpffi lncilpfcgs |
| 301 | getqpfcids ntfdvfvwfg wansslnpii yafnadfrka fstllgcyrl cpatnnaiet |
| 361 | vsinnngaam fsshheprgs iskecnlvyl iphavgssed lkkeeaagia rpleklspal |
| 421 | svildydtdv slekiqpitq ngqhpt |

b) cDNA for NP_{—}000785.

| | |
|---|---|
| 1 | atgaggactc tgaacacctc tgccatggac gggactgggc tggtggtgga gagggacttc |
| 61 | tctgttcgta tcctcactgc ctgtttccta tcgctgctca tcctgtccac gctcctgggg |
| 121 | aacacgctgg tctgtgctgc cgttatcagg ttccgacacc tgcggtccaa ggtgaccaac |
| 181 | ttctttgtca tctccttggc tgtgtcagat ctcttggtgg cagtcctggt catgccctgg |
| 241 | aaggcagtgg ctgagattgc tggcttctgg ccctttgggt ccttctgtaa catctgggtg |
| 301 | gcctttgaca tcatgtgctc cactgcatcc atcctcaacc tctgtgtgat cagcgtggac |
| 361 | aggtattggg ctatctccag ccctttccgg tatgagagaa agatgacccc caaggcagcc |
| 421 | ttcatcctga tcagtgtggc atggaccttg tctgtactca tctccttcat cccagtgcag |
| 481 | ctcagctggc acaaggcaaa acccacaagc ccctctgatg gaaatgccac ttccctggct |
| 541 | gagaccatag acaactgtga ctccagcctc agcaggacat atgccatctc atcctctgta |
| 601 | ataagctttt acatccctgt ggccatcatg attgtcacct acaccaggat ctacaggatt |
| 661 | gctcagaaac aaatacggcg cattgcggcc ttggagaggg cagcagtcca cgccaagaat |
| 721 | tgccagacca ccacaggtaa tggaaagcct gtcgaatgtt ctcaaccgga aagttctttt |
| 781 | aagatgtcct tcaaaagaga aactaaagtc ctgaagactc tgtcggtgat catgggtgtg |
| 841 | tttgtgtgct gttggctacc tttcttcatc ttgaactgca ttttgccctt ctgtgggtct |
| 901 | ggggagacgc agcccttctg cattgattcc aacacctttg acgtgtttgt gtggtttggg |
| 961 | tgggctaatt catccttgaa ccccatcatt tatgccttta atgctgattt tcggaaggca |
| 1021 | ttttcaaccc tcttaggatg ctacagactt tgccctgcga cgaataatgc catagagacg |
| 1081 | gtgagtatca ataacaatgg ggccgcgatg ttttccagcc atcatgagcc acgaggctcc |
| 1141 | atctccaagg agtgcaatct ggtttacctg atcccacatg ctgtgggctc ctctgaggac |
| 1201 | ctgaaaaagg aggaggcagc tggcatcgcc agacccttgg agaagctgtc cccagcccta |
| 1261 | tcggtcatat tggactatga cactgacgtc tetctggaga agatccaacc catcacacaa |
| 1321 | aacggtcagc acccaacctg a |

4. Dopamine D2 Receptor

a) BAC10668. dopamine receptor . . . [gi:22830566] Links LOCUS BAC10668 443 aa linear PRI 28 Aug. 2002 DEFINITION dopamine receptor D2 [Pan troglodytes] Chimpanzee source.

| | |
|---|---|
| 1 | mdplnlswyd ddlerqnwsr pfngsdgkad rphynyyatl ltlliavivf gnvlvcmavs |
| 61 | rekalqtttn ylivslavad llvatlvmpw vvylevvgew kfsrihcdif vfldvmmcta |
| 121 | silnicaisi drytavampm lyntrysskr rvtvmisivw vlsftiscpl lfglnnadqn |
| 181 | eciianpafv vyssivsfyv pfivtllvyi kiyivlrrrr krvntkrssr afrahirapl |
| 241 | kgncthpedm klctvimksn gsfpvnrrrv eaarraqele memlsstspp ertryspipp |
| 301 | sbhqltlpdp shhglhstpd spakpekngh akdhpkiaki feiqtmpngk trtslktmsr |
| 361 | rklsqqkekk atqmlaivlg vfiicwlpff ithilnihcd cnippvlysa ftwlgyvnsa |
| 421 | vnpiiyttfn lefrkaflki lhc |

b) cDNA of Bac10668

| | |
|---|---|
| 1 | atggatccac tgaatctgtc ctggtatgat gatgatctgg agaggcagaa ctggagccgg |
| 61 | cccttcaacg ggtcagacgg gaaggcggac agaccccact acaactacta tgccacactg |
| 121 | ctcaccctgc tcatcgctgt cattgtcttc ggcaacgtgc tggtgtgcat ggctgtgtcc |
| 181 | cgcgagaagg cgctgcagac caccaccaac tacctgatcg tcagcctcgc agtggccgac |
| 241 | ctcctcgtcg ccacactggt catgccctgg gttgtctacc tggaggtggt aggtgagtgg |
| 301 | aaattcagca ggattcactg tgacatcttc gtcactctgg acgtcatgat gtgcacggcg |
| 361 | agcatcctga acttgtgtgc catcagcatc gacaggtaca cagctgtggc catgcccatg |
| 421 | ctgtacaata cgcgctacag ctccaagcgc cgggtcaccg tcatgatctc catcgtctgg |
| 481 | gtcctgtcct tcaccatctc ctgcccactc ctcttcggac tcaataacgc agaccagaac |
| 541 | gagtgcatca ttgccaaccc ggccttcgtg gtctactcct ccatcgtctc cttctacgtg |
| 601 | cccttcattg tcaccctgct ggtctacatc aagatctaca ttgtcctccg cagacgccgc |
| 661 | aagcgagtca acaccaaacg cagcagccga gctttcaggg cccacctgag ggctccacta |
| 721 | aagggcaact gtactcaccc cgaggacatg aaactctgca ccgttatcat gaagtctaat |
| 781 | gggagtttcc cagtgaacag gcggagagtg gaggctgccc ggcgagccca ggagctggag |
| 841 | atggagatgc tctccagcac cagcccaccc gagaggaccc ggtacagccc catcccaccc |
| 901 | agccaccacc agctgactct ccccgaccca tcccaccacg gtctccacag cactcccgac |
| 961 | agccccgcca aaccagagaa gaatgggcat gccaaagacc accccaagat tgccaagatc |
| 1021 | tttgagatcc agaccatgcc caatggcaaa acccggacct ccctcaagac catgagccgt |
| 1081 | aggaagctct cccagcagaa ggagaagaaa gccactcaga tgctcgccat tgttctcggc |
| 1141 | gtgttcatca tctgctggct gcccttcttc atcacacaca tcctgaacat acactgtgac |
| 1201 | tgcaacatcc cgcctgtcct gtacagcgcc ttcacgtggc tgggctatgt caacagcgcc |
| 1261 | gtgaacccca tcatctacac caccttcaac attgagttcc gcaaggcctt cctgaagatc |
| 1321 | ctccactgct ga |

c) 2. NP_{—}036679. dopamine receptor . . . [gi:6978777] Links LOCUS Drd2 444 aa linear ROD 27 Aug. 2002 DEFINITION dopamine receptor D2 [Rattus norvegicus].

| | |
|---|---|
| 1 | mdplnlswyd ddlerqnwsr pfngsegkad rphynyyaml ltllifiivf gnvlvcmavs |
| 61 | rekalqfttn ylivslavad llvatlvmpw vvylevvgew kfsrihcdif vtldvmmcta |
| 121 | silnlcaisi drytavampm lyntrysskr rvtvmiaivw vlsftiscpl lfglnntdqn |
| 181 | eciianpafv vyssivsfyv pfivtllvyi kiyivlrkrr krvntkrssr afranlktpl |
| 241 | kgncthpedm klctvimksn gsfpvnrrrm daarraqele memlsstspp ertryspipp |
| 301 | shhqltlpdp shhglhsnpd spakpekngh akivnpriak ffeiqtmpng ktrtslktms |
| 361 | rrklsqqkek katqmlaivl gvfiicwlpf fithilnihc dcnippvlys aftwlgyvns |
| 421 | avnpiiyttf niefrkafmk ilhc |

d) cDNA for NP_{—}036679

| | |
|---|---|
| 1 | atggatccac tgaacctgtc ctggtacgat gacgatctgg agaggcagaa ctggagccgg |
| 61 | cccttcaatg ggtcagaagg gaaggcagac aggccccact acaactacta tgccatgctg |
| 121 | ctcaccctcc tcatctttat catcgtcttt ggcaatgtgc tggtgtgcat ggctgtatcc |
| 181 | cgagagaagg ctttgcagac caccaccaac tacttgatag tcagccttgc tgtggctgat |
| 241 | cttctggtgg ccacactggt aatgccgtgg gttgtctacc tggaggtggt gggtgagtgg |
| 301 | aaattcagca ggattcactg tgacatcttt gtcactctgg atgtcatgat gtgcacagca |
| 361 | agcatcctga acctgtgtgc catcagcatt gacaggtaca cagctgtggc aatgcccatg |
| 421 | ctgtataaca cacgctacag ctccaagcgc cgagttactg tcatgattgc cattgtctgg |
| 481 | gtcctgtcct tcaccatctc ctgcccactg ctcttcggac tcaacaatac agaccagaat |
| 541 | gagtgtatca ttgccaaccc tgcctttgtg gtctactcct ccattgtctc attctacgtg |
| 601 | cccttcatcg tcactctgct ggtctatatc aaaatctaca tcgtcctccg gaagcgccgg |
| 661 | aagcgggtca acaccaagcg cagcagtcga gctttcagag ccaacctgaa gacaccactc |
| 721 | aagggcaact gtacccaccc tgaggacatg aaactctgca ccgttatcat gaagtctaat |
| 781 | gggagtttcc cagtgaacag gcggagaatg gatgctgccc gccgagctca ggagctggaa |
| 841 | atggagatgc tgtcaagcac cagtccccca gagaggaccc ggtatagccc catccctccc |
| 901 | agtcaccacc agctcactct ccctgatcca tcccaccacg gcctacatag caaccctgac |
| 961 | agtcctgcca aaccagagaa gaatgggcac gccaagattg tcaatcccag gattgccaag |
| 1021 | ttctttgaga tccagaccat gcccaatggc aaaacccgga cctcccttaa gacgatgagc |
| 1081 | cgcagaaagc tctcccagca gaaggagaag aaagccactc agatgcttgc cattgttctc |
| 1141 | ggtgtgttca tcatctgctg gctgcccttc ttcatcacgc acatcctgaa tatacactgt |
| 1201 | gattgcaaca tcccaccagt cctctacagc gccttcacat ggctgggcta tgtcaacagt |
| 1261 | gccgtcaacc ccatcatcta caccaccttc aacatcgagt tccgcaaggc cttcatgaag |
| 1321 | atcttgcact gctga |

e) 3. P13953. D(2) dopamine rec . . . [gi:118207] Links LOCUS D2DR_MOUSE 444 aa linear ROD 16 Oct. 2001 DEFINITION D(2) dopamine receptor

| | |
|---|---|
| 1 | mdplnlswyd ddlerqnwsr pfngsegkad rphynyyaml ltllifiivf gnvlvcmavs |
| 61 | rekalqtttn ylivslavad llvatlvmpw vvylevvgew kfsrihcdif vtldvmmcta |
| 121 | silnlcaisi drytavampm lyntrysskr rvtvmiaivw vlsftiscpl lfglnntdqn |
| 181 | eciianpafv vyssivsfyv pfivtllvyi kiyivlrkrr krvntkrssr afranlktpl |
| 241 | kgncthpedm klctvimksn gsfpvnrrrm daarraqele memlsstspp ertryspipp |
| 301 | shhqltlpdp shhglhsnpd spakpekngh akivnpriak ffeiqtmpng ktrtslktms |
| 361 | rrklsqqkek katqmlaivl gvfiicwlpf fithilnihc dcnippvlys aftwlgyvns |
| 421 | avnpiiyttf niefrkafmk ilhc |

5. DAT Dopamine Transporter Sequences

a) Q61327. Sodium-dependent . . . [gi:21264519] Links LOCUS S6A3_MOUSE 619 aa linear ROD 15 Jun. 2002 DEFINITION Sodium-dependent dopamine transporter (DA transporter) (DAT).

| | |
|---|---|
| 1 | mskskcsvgp mssvvapake pnavgpreve lilvkeqngv qltnstlinp pqtpvevqer |
| 61 | etwskkidfl lsvigfavdl anvwrfpylc ykngggaflv pyllfmviag mplfymelal |
| 121 | gqfnregaag vwkicpvlkg vgftvilisf yvgffyvnii awalhyffss flmdlpwihc |
| 181 | nntwnspncs dahssnssdg lglndtfgtt paaeyfcrgv lhlhqsrgid dlgpprwqlt |
| 241 | aclvlvivll yfslwkgvkt sgkvvwitat mpyvvltall lrgvtlpgam dgiraylsvd |
| 301 | fyrlceasvw idaatqvcfs lgvgfgvlia fssynkftnn cyrdaiitts insltsfssg |
| 361 | fvvfsflgym aqkhnvpird vatdgpglif iiypeaiatl plssawaavf flmlltlgid |
| 421 | samggmesvi tglvdefqll hrhrelftlg ivlatfllsl fcvtnggiyv ftlldhfaag |
| 481 | tsilfgvlie aigvawfygv qqfsddikqm tgqrpnlywr lcwklvspcf llyvvvvsiv |
| 541 | tfrpphygay ifpdwanalg wiiatssmam vpiyatykfc slpgsfrekl ayaitpekdr |
| 601 | qlvdrgevrq ftlrhwllv |

b) P23977. Sodium-dependent . . . [gi:128613] Links LOCUS S6A3_RAT 619 aa linear ROD 16 Oct. 2001 DEFINITION Sodium-dependent dopamine transporter (DA transporter) (DAT)

| | |
|---|---|
| 1 | mskskcsvgp mssvvapake snavgpreve lilvkeqngv qltnstlinp pqtpveaqer |
| 61 | etwskkidfl lsvigfavdl anvwrfpylc ykngggaflv pyllfmviag mplfymelal |
| 121 | gqfnregaag vwkicpvlkg vgftvilisf yvgffynvii awalhyffss ftmdlpwihc |
| 181 | nntwnspncs dahasnssdg lglndtfgtt paaeyfergv lhlhqsrgid dlgpprwqlt |
| 241 | aclvlvivll yfslwkgvkt sgkvvwitat mpyvvltall lrgvtlpgam dgiraylsvd |
| 301 | fyrlceasvw idaatqvcfs lgvgfgvlia fssynkftnn cyrdaiitts insltsfssg |
| 361 | fvvfsflgym aqkhnvpird vatdgpglif iiypeaiatl plssawaavf flmlltlgid |
| 421 | samggmesvi tglvdefqll hrhrelftlg ivlatfllsi fcvtnggiyv ftlldhfaag |
| 481 | tsilfgvlie aigvawfygv qqfsddikqm tgqrpnlywr lcwklvspcf llyvvvvsiv |
| 541 | tfrpphygay ifpdwanalg wiiatssmam vpiyatykfc slpgsfrckl ayaitpekdh |
| 601 | qlvdrgevrq ftlrhwlll |

c) Q01959. Sodium-dependent . . . [gi:266667] Links LOCUS S6A3_HUMAN 620 aa linear PRI 16 Oct. 2001 DEFINITION Sodium-dependent dopamine transporter (DA transporter) (DAT).

| | |
|---|---|
| 1 | mskskcsvgl mssvvapake pnavgpkeve lilvkeqngv qltsstltnp rqspveaqdr |
| 61 | etwgkkidfl lsvigfavdl anvwrfpylc ykngggaflv pyllfmviag mplfymelal |
| 121 | gqfnregaag vwkicpilkg vgftvilisl yvgffynvii awalhylfss fttelpwihc |
| 181 | nnswnspncs dahpgdssgd ssglndtfgt tpaaeyferg vlhlhqshgi ddlgpprwql |
| 241 | taclvlvivl lyfslwkgvk tsgkvvwita tmpyvvltal llrgvtlpga idgiraylsv |
| 301 | dfyrlceasv widaatqvcf slgygfgvli afssynkftn ncyrdaivtt sinsltsfss |
| 361 | gfvvfsflgy maqkhsvpig dvakdgpgli fijypeajat lplssawavv ffimlltlgi |
| 421 | dsamggmesv itglidefql lhrhrelftl fivlatfils lfcvtnggiy vfthldhfaa |
| 481 | gtsilfgvli eaigvawfyg vgqfsddiqq mtgqrpslyw rlcwklvspc fllfvvvvsi |
| 541 | vtfrpphyga yifpdwanal gwviatssma mvpiyaaykf cslpgsfrek layaiapekd |
| 601 | relvdrgevr qftlrhwlkv |

d) AF109391. Mus musculus dopa . . . [gi:9230265] Links LOCUS AF109391 1873 bp mRNA linear ROD 16 Jul. 2000 DEFINITION Mus musculus dopamine transporter (Dat) mRNA, complete cds.

| | |
|---|---|
| 1 | tacccatgag taaaagcaaa tgctccgtgg gaccaatgtc ttctgtggtg gccccggcta |
| 61 | aagagcccaa tgctgtgggc cccagagagg tggagctcat cttggtcaag gagcagaatg |
| 121 | gagtgcagct gaccaattcc accctcatca acccaccgca gacaccagtg gaggttcaag |
| 181 | agcgggagac ctggagcaag aaaatcgatt tcctgctctc agtcatcggc ttcgctgtgg |
| 241 | acctggccaa tgtttggagg tttccctacc tgtgctacaa aaatggtgga ggtgccttcc |
| 301 | tggtgcccta cctgctcttc atggttattg ccgggatgcc cctcttctac atggagctgg |
| 361 | ctctcgggca gttcaacaga gaaggagctg ctggtgtctg gaagatctgc cctgtcctga |
| 421 | aaggtgtggg cttcactgtc atcctcatct ctttctacgt gggcttcttc tacaatgtca |
| 481 | tcattgcatg ggcactgcac tacttcttct cctccttcac catggacctc ccatggatcc |
| 541 | actgcaacaa cacctggaac agccccaact gttctgatgc acatagcagc aactctagcg |
| 601 | atggcctggg cctcaacgac acctttggga ccacacccgc tgctgagtat tttgagcgtg |
| 661 | gtgtgctgca cctccatcag agtcgtggca ttgatgacct gggccctcca cggtggcagc |
| 721 | tcacagcctg cctggtgctg gtcattgttc tgctctactt cagcctgtgg aagggagtaa |
| 781 | agacttcagg gaaggtggtg tggatcacag ctaccatgcc ctatgtagtc ctcacagccc |
| 841 | tgctcctgcg tggagtcacc ctccctgggg ccatggatgg catcagagca tacctcagtg |
| 901 | tggacttcta ccgtctctgt gaggcatctg tgtggatcga tgccgccacc caggtgtgct |
| 961 | tctcccttgg cgttgggttt ggggtgctga ttgccttctc cagttacaat aagttcacca |
| 1021 | ataactgcta tagagatgca atcatcacca cctccattaa ctccctgacg agcttctcct |
| 1081 | ctggcttcgt tgtcttctcc ttcctggggt acatggcaca gaagcacaat gtgcccatca |
| 1141 | gggatgtggc cacagatgga cctgggttga tcttcatcat ctaccctgag gcaatcgcca |
| 1201 | cactcccgct gtcttcagcc tgggccgctg tcttcttcct catgctgctc actctgggta |
| 1261 | tcgacagtgc catggggggc atggagtctg tgatcactgg gcttgtcgat gagttccagc |
| 1321 | tgctacatcg gcatcgagag ctcttcactc ctggcattgt cctggctact ttcctgctgt |
| 1381 | ctctcttctg tgtcaccaac ggtggcatct atgtcttcac actgctggac cactttgcag |
| 1441 | ctggcacatc tatcctcttt ggagtgctca ttgaagccat tggggtggcc tggttctacg |
| 1501 | gtgtccagca attcagtgat gacatcaagc agatgactgg gcagcgaccc aacctgtact |
| 1561 | ggcggctatg ctggaagctg gtcagcccct gcttccttct gtatgtggtc gtggtcagca |
| 1621 | ttgtgacctt cagaccccca cactatggag cctacatctt cccagactgg gccaatgccc |
| 1681 | tgggctggat cattgccaca tcctccatgg ccatggtgcc catttatgcc acctataagt |
| 1741 | tctgcagcct gccagggtcc ttccgagaga aactggccta tgccatcaca cctgagaaag |
| 1801 | accgccagct agtggacaga ggggaggtgc gccaattcac gctgcgccat tggctgttgc |
| 1861 | tgtaaagtgg aag |

e) S44626. dopamine transpor . . . [gi:256312] Links LOCUS S44626 2020 bp mRNA linear PRI 8 May 1993 DEFINITION dopamine transporter [human, substantia nigra, mRNA, 2020 nt].

| | |
|---|---|
| 1 | gaattcctca actcccagtg tgcccatgag taagagcaaa tgctccgtgg gactcatgtc |
| 61 | ttccgtggtg gccccggcta aggagcccaa tgccgtgggc ccgaaggagg tggagctcat |
| 121 | ccttgtcatg gagcagaacg gagtgcagct caccagctcc accctcacca acccgcggca |
| 181 | gagccccgtg gaggcccagg atcgggagac ctggggcaag aagatcgact ttctcctgtc |
| 241 | cgtcattggc tttgctgtgg acctggccaa cgtctggagg ttcccctacc tgtgctacaa |
| 301 | aaatggtggc ggtgccttcc tggtccccta cctgctcttc atggtcattg ctgggatgcc |
| 361 | acttttctac atggagctgg ccctcggcca gttcaacagg gaaggggccg ctggtgtctg |
| 421 | gaagatctgc cccatactga aaggtgtggg cttcacggtc atcctcatct CaCtgtatgt |
| 481 | cggcttcttc tacaacgtca tcatcgcctg ggcgctgcac tatctcttct cctccttcac |
| 541 | cacggagctc ccctggatcc actgcaacaa ctcctggaac agccccaact gctcggatgc |
| 601 | ccatcctggt gactccagtg gagacagctc gggcctcaac gacacttttg ggaccacacc |
| 661 | tgctgccgag tactttgaac gtggcgtgct gcacctccac cagagccatg gcatcgacga |
| 721 | cctggggcct ccgcggtggc agctcacagc ctgcctggtg ctggtcatcg tgctgctgta |
| 781 | cttcagcctc tggaagggcg tgaagacctc agggaaggtg gtatggatca cagccaccat |
| 841 | gccatacgtg gtcctcactg ccctgctcct gcgtggggtc accctccctg gagccataga |
| 901 | cggcatcaga gcatacctga gcgttgactt ctaccggctc tgcgaggcgt ctgtttggat |
| 961 | tgacgcggcc acccaggtgt gcttctccct gggcgtgggg ttcggggtgc tgatcgcctt |
| 1021 | ctccagctac aacaagttca ccaacaactg ctacagggac gcgattgtca ccacctccat |
| 1081 | caactgcctg acgagcttct cctccggctt cgtcgtcttc tccttcctgg ggtacatggc |
| 1141 | acagaagcac agtgtgccca tcggggacgt ggccaaggac gggccagggc tgatcttcat |
| 1201 | catctacccg gaagccatcg ccacgctccc tctgtcctcg gcctgggcgg tggtcttctt |
| 1261 | catcatgctg ctcaccctgg gtatcgacag cgccatgggt ggtatggagt cagtgatcac |
| 1321 | cgggctcatc gatgagttcc agctgctgca cagacaccgt gagctcttca cgctcttcat |
| 1381 | cgtcctggcg accttcctcc tgtccctgtt ctgcgtcacc aacggtggca tctacgtctt |
| 1441 | cacgctcctg gaccattttg cagccggcac gtccatcctc tttggagtgc tcatcgaagc |
| 1501 | catcggagtg gcctggttct atggtgttgg gcagttcagc gacgacatcc agcagatgac |
| 1561 | cgggcagcgg cccagcctgt actggcggct gtgctggaag ctggtcagcc cctgctttct |
| 1621 | cctgttcgtg gtcgtggtca gcattgtgac cttcagaccc ccccactacg gagcctacat |
| 1681 | cttccccgac tgggccaacg cgctgggctg ggtcatcgcc acatcctcca tggccatggt |
| 1741 | gcccatctat gcggcctaca agttctgcag cctgcctggg tcctttcgag agaaactggc |
| 1801 | ctacgccatt gcacccgaga aggaccgtga gctggtggac agaggggagg tgcgccagtt |
| 1861 | cacgctccgc cactggctca aggtgtagag ggagcagaga cgaagacccc aggaagtcat |
| 1921 | cccgcaatgg gagagacacg aacaaaccaa ggaaatctaa gtttcgagag aaaggagggg |
| 1981 | caacttctac tcttcaacct cttactgaaa acacaaacca |

What is claimed is:

1. A method for treating Parkinson's disease, comprising administering to a subject a compound having the formula I 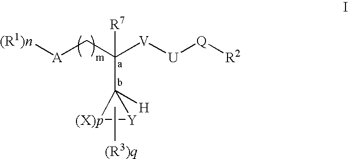
wherein
A represents a cycloalkyl group, heterocycloalkyl group, an aryl group or heteroaryl group;
U is absent or when U is present, U represents —C(═O)—, —C(═S)—, —P(═O)(OR⁵)—, —S(O₂)— or —S(O)—;
V is absent or when V is present, V is NR⁶, O or S;
Q is absent or when Q is present, V is NR⁶, O or S;
Y represents NR⁴, O or S;
X is, independently, C, N, S, Se or O;
R¹ is, independently, hydrogen, aryl, alkyl, alkoxy, hydroxy, hydroxyalkyl, aralkyl, halogen, cyano, aldehyde, ketone, ester, carbonate, amido, amino, alkylamino, nitro, thiol, thioalkyl or a sulfo-oxo group;
R² is hydrogen, aryl, alkyl, aralkyl, alkoxy, hydroxy, hydroxyalkyl, halogen, ester, carbonate, amido, amino, alkylamino, thiol or thioalkyl;
R³ is, independently, hydrogen, aryl, alkyl, aralkyl, alkoxy, hydroxy, hydroxyalkyl, halogen, cyano, aldehyde, ketone, ester, carbonate, amido, amino, alkylamino, nitro or a sulfo-oxo group;
wherein the ring formed by X, Y and carbon b optionally contains a carbon-carbon double or carbon-oxygen double bond;
R⁴ is hydrogen, alkyl, keto, aryl, aralkyl, heteroaryl or heteroaralkyl;
R⁵, R⁶ and R⁷ are, independently, hydrogen, alkyl, alkenyl, aryl, heteroaryl, aralkyl or heteroaralkyl;
m is an integer of from 0 or 1;
n is an integer of from 0 to 7;
p is an integer of from 3 to 6;
q is an integer of from 0 to 16;
the stereochemistry at carbon a and carbon b is R or S;
or a pharmaceutically acceptable salt, pro-drug or metabolite thereof.

2. The method of claim 1, wherein the compound has the formula II 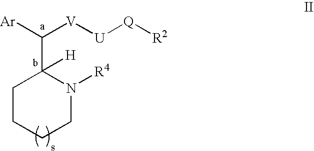
wherein
U is absent or when U is present, U represents —C(═O)—, —C(═S)—, —P(═O)(OR⁵)—, —S(O₂)— or —S(O)—;
V is absent or when V is present, V is NR⁶, O or S;
Q is absent or when Q is present, V is NR⁶, O or S;
R² is hydrogen, aryl, alkyl, aralkyl, alkoxy, hydroxy, hydroxyalkyl, halogen, ester, carbonate, amido, amino, alkylamino, thiol or thioalkyl;
R⁵ and R⁶ are, independently, hydrogen, alkyl, alkenyl, aryl, heteroaryl, aralkyl or heteroaralkyl;
s is an integer of from 0 to 2;
Ar is a substituted or unsubsituted aryl or heteroaryl group;
the stereochemistry at carbon a and carbon b is R or S;
or a pharmaceutically acceptable salt, pro-drug or metabolite thereof.

3. The method of claim 1, wherein the compound has the formula III 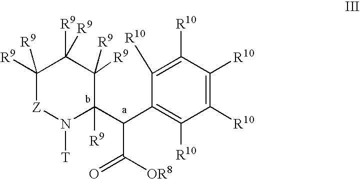
wherein
R⁸ is hydrogen, aryl, alkyl, alkenyl, hydroxyalkyl, aralkyl, aldehyde, ketone, cycloalkyl, heteroaryl or the pharmaceutically acceptable salt thereof;
R⁹ and R¹⁰ are, independently, hydrogen, aryl, alkyl, alkenyl, alkoxy, hydroxy, hydroxyalkyl, aralkyl, halogen, cyano, aldehyde, ketone, ester, carbonate, amido, amino, alkylamino, thiol, thioalkyl, nitro or a sulfo-oxo group;
Z is —CH₂— or —C(═O)—;
T is hydrogen or —C(═O)—N(R¹¹)₂, wherein R¹¹ is, independently, hydrogen, aryl, alkyl or aralkyl;
and the stereochemistry at carbon a and carbon b is R or S,
or a pharmaceutically acceptable salt, pro-drug or metabolite thereof.

4. The method of claim 3, wherein each R⁹ is hydrogen and each R¹⁰ is hydrogen.

5. The method of claim 3, wherein Z is CH₂ and T is hydrogen.

6. The method of claim 3, wherein R⁸ is hydrogen or C₁-C₈ alkyl.

7. The method of claim 3, wherein R⁸ is methyl.

8. The method of claim 3, wherein the stereochemistry at carbons a and b is R.

9. The method of claim 3, wherein R⁸ is C₁-C₈ alkyl, each R⁹ is hydrogen, each R¹⁰ is hydrogen, Z is CH₂, T is hydrogen, and the stereochemistry at carbons a and b is R.

10. The method of claim 3, wherein R⁸ is methyl, each R⁹ is hydrogen, each R¹⁰ is hydrogen, Z is CH₂, T is hydrogen, and the stereochemistry at carbons a and b is R.

11. A method for treating Parkinson's disease, comprising administering to a subject d-threo methylphenidate.

12. A method for treating Parkinson's disease, comprising administering to a subject a compound having the formula IV 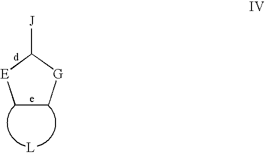
wherein
when d is a single bond, E is S, O, C(R¹¹)₂, or NR¹¹, and when d is double bond, E is CR¹¹ or N;
G is S, O, C(R¹¹)₂, or NR¹¹;
J is hydrogen, C(R¹²)₃, SR¹², OR¹², or N(R¹²)₂;
wherein R¹¹ and R¹² are, independently, hydrogen, aryl, alkyl, aralkyl, alkoxy, hydroxy, hydroxyalkyl, halogen, ester, carbonate, amido, amino, alkylamino, thiol or thioalkyl;
L is a fused substituted or unsubstituted cycloalkyl group, heterocycloalkyl group, aryl group, or heteroaryl group;
d is a single bond or a double bond; and
e is a single bond or a double bond.

13. The method of claim 12, wherein d is a double bond and E is N.

14. The method of claim 12, wherein G is S.

15. The method of claim 12, wherein L is a cycloalkyl group.

16. The method of claim 15, wherein the cycloalkyl group is a cyclohexyl group having at least one substituted or unsubstituted amino group.

17. The method of claim 16, wherein the amino group is NHPr.

18. The method of claim 12, wherein J is N(R¹²)₂.

19. The method of claim 18, wherein each R¹² is hydrogen.

20. The method of claim 12, wherein d and e are double bonds.

21. The method of claim 12, wherein the compound has the formula V

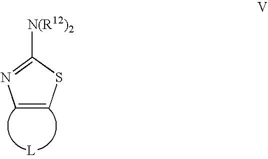

22. The method of claim 12, wherein the compound has the formula VI 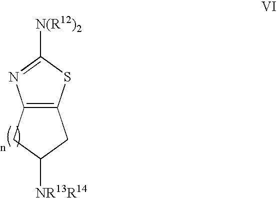
wherein R¹³ and R¹⁴ are hydrogen, aryl, alkyl, aralkyl, hydroxyalkyl, or R¹³ and R¹⁴ form a cycloalkyl group or heterocycloalkyl group, and n is from 0 to 3.

23. A method for treating Parkinson's disease, comprising administering to a subject a compound having the formula VII 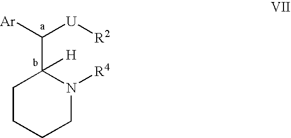
wherein
U is —C(═O)—, —S(O₂)— or —S(O)—;
R² is hydrogen, aryl, aralkyl;
R⁴ is hydrogen, alkyl, keto, aryl, aralkyl, heteroaryl or heteroaralkyl; and
the stereochemistry at carbon a and carbon b is R or S.

24. A method for treating Parkinson's disease, comprising administering to a subject a compound having the formula VIII 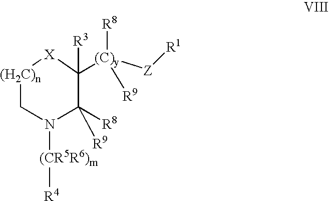
wherein
X represents C(R³)₂, O, S, SO, SO₂, NR², NC(O)R⁷, NC(O)OR², NS(O)₂R⁷, or C═O;
Z represents C(R³)₂, C(O), O, NR, NC(O)OR, S, SO, or SO₂;
m is 1, 2, 3, 4 or 5;
n is 1 or 2;
p is 0, 1, 2, or 3;
y is 0, 1, or 2;
R¹ represents H, alkyl, cycloalkyl, aryl, heteroaryl, aralkyl, or heteroaralkyl;
R¹ represents H, alkyl, aryl, heteroaryl, aralkyl, or heteroaralkyl;
R and R¹ may be connected through a covalent bond;
R² represents independently for each occurrence H, alkyl, fluoroalkyl, aryl, heteroaryl, or cycloalkyl;
R³ represents independently for each occurrence H, alkyl, aryl, OR², OC(O)R², CH₂OR², or CO₂R²; wherein any two instances of R³ may be connected by a covalent tether whose backbone consists of 1, 2, 3, or 4 carbon atoms;
R⁴ represents independently for each occurrence H, alkyl, cycloalkyl, aryl, heteroaryl, alkenyl, or OR;
R⁵ and R⁶ are selected independently for each occurrence from the group consisting of H, alkyl, (CH₂)ₚY, aryl, heteroaryl, F, OR², and OC(O)R²; or an instance of CR⁵R⁶ taken together is C(O);
R⁷ represents alkyl, cycloalkyl, aryl, heteroaryl, aralkyl, or heteroaralkyl;
R⁸ and R⁹ are selected independently for each occurrence from the group consisting of H, alkyl, (CH₂)ₚY, aryl, heteroaryl, F, OR², and OC(O)R²; or an instance of CR⁸R⁹ taken together is C(O);
Y represents independently for each occurrence OR², N(R²)₂, SR₂, S(O)R², S(O)₂R², or P(O)(OR²)₂; any two instances of R² may be connected through a covalent bond; a covalent bond may connect R⁴ and an instance of R⁵ or R⁶; any two instances of R⁵ and R⁶ may be connected through a covalent bond;
any two geminal or vicinal instances of R⁸ and R⁹ may be connected through a covalent bond;
and the stereochemical configuration at any stereocenter of a compound represented by A is R, S, or a mixture of these configurations.

25. A method for treating Parkinson's disease, comprising administering to a subject a [A] polypharmacophore having the formula IX: 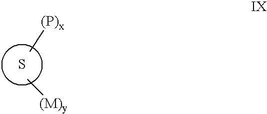
wherein S comprises a scaffold unit; P comprises a pharmacophore unit, wherein x is greater than or equal to two; and M comprises a modifier unit, wherein y is greater than or equal to 0, whereby each one of P and M, for each occurrence, is appended to said scaffold unit, and whereby the polypharmacophore interacts with at least two biological targets.

26. A labeled compound comprising any one of compounds in claims 1-25.

27. A method for treating anxiety, autism, depression, sexual dysfunction, hypertension, migraine, Alzheimer's disease, obesity, emesis, psychosis, analgesia, schizophrenia, Parkinson's disease, Huntington's disease, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, or Tourette's syndrome comprising administering to a subject a compound recited in any one of claims 1-25.

28. The method of claim 1, wherein the compound is administered in an amount between 5 and 40 mg/kg.

29. A pharmaceutical formulation having the formula I 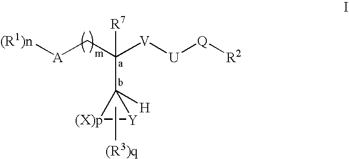
wherein
A represents a cycloalkyl group, heterocycloalkyl group, an aryl group or heteroaryl group;
U is absent or when U is present, U represents —C(═O)—, —C(═S)—, —P(═O)(OR⁵)—, —S(O₂)— or —S(O)—;
V is absent or when V is present, V is NR⁶, O or S;
Q is absent or when Q is present, V is NR⁶, O or S;
Y represents NR⁴, O or S;
X is, independently, C, N, S, Se or O;
R¹ is, independently, hydrogen, aryl, alkyl, alkoxy, hydroxy, hydroxyalkyl, aralkyl, halogen, cyano, aldehyde, ketone, ester, carbonate, amido, amino, alkylamino, nitro, thiol, thioalkyl or a sulfo-oxo group;
R² is hydrogen, aryl, alkyl, aralkyl, alkoxy, hydroxy, hydroxyalkyl, halogen, ester, carbonate, amido, amino, alkylamino, thiol or thioalkyl;
R³ is, independently, hydrogen, aryl, alkyl, aralkyl, alkoxy, hydroxy, hydroxyalkyl, halogen, cyano, aldehyde, ketone, ester, carbonate, amido, amino, alkylamino, nitro or a sulfo-oxo group;
wherein the ring formed by X, Y and carbon b optionally contains a carbon-carbon double or carbon-oxygen double bond;
R⁴ is hydrogen, alkyl, keto, aryl, aralkyl, heteroaryl or heteroaralkyl;
R⁵, R⁶ and R⁷ are, independently, hydrogen, alkyl, alkenyl, aryl, heteroaryl, aralkyl or heteroaralkyl;
m is an integer of from 0 or 1;
n is an integer of from 0 to 7;
p is an integer of from 3 to 6;
q is an integer of from 0 to 16;
the stereochemistry at carbon a and carbon b is R or S;
or a pharmaceutically acceptable salt, pro-drug or metabolite thereof;
wherein the pharmaceutical composition is in an amount of about 5 to 40 mg/kg.


SEQUENCE LISTING (18 sequences)

SEQ ID NO: 1 - protein, 515 aa, Artificial Sequence
  Description of Artificial Sequence/note = synthetic construct
MALSDLVLLRWLRDSRHSRKLILFIVFLALLLDNMLLTVVVPIIPSYLYSIKHEKNSTEIQTTRPELVVSTSESIFSYYNNSTVLITGNATGTLPGGQSHKATSTQHTVANTTVPSDCPSEDRDLLNENVQVGLLFASKATVQLLTNPFIGLLTNRIGYPIPMFAGFCIMFISTVMFAFSSSYAFLLIARSLQGIGSSCSSVAGMGMLASVYTDDEERGNAMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMVIVGISILCIPFAKNIYGLIAPNFGVGFAIGMVDSSMMPIMGYLVDLRHVSVYGSVYAIADVAFCMGYAIGPSAGGAIAKAIGFPWLMTIIGIIDIAFAPLCFFLRSPPAKEEKMAILMDHNCPIKTKMYTQNNVQSYPIGDDEESESD

SEQ ID NO: 2 - DNA, 1548 bp, Artificial Sequence
  Description of Artificial Sequence/note = synthetic construct
atggccctgagcgatctggtgctgctgcgatggctgcgggacagccgccactcgcgcaaactgatcctgttcatcgtgttccttgcgctgctgctggacaacatgctgctcaccgtcgtggttcccatcatccccagctatctgtacagcattaagcatgagaaaaactctacggaaatccagaccaccagaccagagctcgtggtctccacctccgaaagcatcttctcttactataacaactctactgtgttgatcaccgggaatgccactgggactcttccaggagggcagtcacacaaggctaccagcacacagcacactgtggctaacaccactgtcccttcggactgtcccagtgaagacagagaccttctgaatgagaatgtgcaagttgggctgctgtttgcctccaaagccactgtccagctcctcactaacccattcataggacttctgaccaacagaattggctatccaattcccatgtttgccggcttctgcatcatgtttatctcaacagttatgtttgccttctccagcagctatgccttcctgctgatcgccaggtcccttcagggaattggctcctcctgctcatccgtggctgggatgggtatgctggccagcgtgtacacagatgatgaggagagggggaacgccatgggcattgctttgggtggcctggccatgggagtcttagtgggaccccccttcgggagtgtgctctatgagtttgtggggaagacagctcccttcctggtgctagctgccttggtgctcttggatggggctattcagctctttgtgctccagccgtcccgagtacagccagagagtcagaaggggacacctctaacgaccttgctgaaggatccatacatcctcatcgctgcaggctccatctgctttgcaaacatggggatagccatgctggagcccgccctgcccatctggatgatggagaccatgtgttcccgaaagtggcagctgggcgttgctttcctcccggcgagcatctcttatctcattggaaccaatatttttgggatacttgcacacaaaatgggaaggtggctatgtgctcttctgggaatggtaattgttggaatcagcattttatgcatcccctttgcaaaaaatatctatggactcatcgctcccaactttggagttggttttgcaattgggatggtggactcctctatgatgcctatcatgggctacctggttgacctgcggcatgtgtctgtctatgggagtgtttatgccattgcagacgtggccttttgtatgggctatgctatcggtccctctgctggtggtgccatcgcaaaggcaattggctttccttggcttatgacaattattgggataattgatatcgcttttgctccactctgctttttccttcgaagtccacctgctaaggaggaaaaaatggctatcctcatggaccacaactgtcccattaaaacaaagatgtacactcagaataatgtccagtcatatcccatcggtgatgatgaagaatctgaaagtgactga

SEQ ID NO: 3 - protein, 514 aa, Artificial Sequence
  Description of Artificial Sequence/note = synthetic construct
MALSELALVRWLQESRHSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIAPNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 4 - DNA, 1545 bp, Artificial Sequence
  Description of Artificial Sequence/note = synthetic construct
atggccctgagcgagctggcgctggtccgctggctgcaggagagccgccactcgcggaagctcatcctgttcatcgtgttcctggcgctgctgctggacaacatgctgctcactgtcgtggtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacagaattggctatccaattcccatatttgcgggattctgcatcatgtttgtctcaacaattatgtttgccttctccagcagctatgccttcctgctgattgccaggtcgctgcagggcatcggctcgtcctgctcctctgtggctgggatgggcatgcttgccagtgtctacacagatgatgaagagagaggcaacgtcatgggaatcgccttgggaggcctggccatgggggtcttagtgggcccccccttcgggagtgtgctctatgagtttgtggggaagacggctccgttcctggtgctggccgccctggtactcttggatggagctattcagctctttgtgctccagccgtcccgggtgcagccagagagtcagaaggggacacccctaaccacgctgctgaaggacccgtacatcctcattgctgcaggctccatctgctttgcaaacatgggcatcgccatgctggagccagccctgcccatctggatgatggagaccatgtgttcccgaaagtggcagctgggcgttgccttcttgccagctagtatctcttatctcattggaaccaatatttttgggatacttgcacacaaaatggggaggtggctttgtgctcttctgggaatgataattgttggagtcagcattttatgtattccatttgcaaaaaacatttatggactcatagctccgaactttggagttggttttgcaattggaatggtggattcgtcaatgatgcctatcatgggctacctcgtagacctgcggcacgtgtccgtctatgggagtgtgtacgccattgcggatgtggcattttgtatggggtatgctataggtccttctgctggtggtgctattgcaaaggcaattggatttccatggctcatgacaattattgggataattgatattctttttgcccctctctgcttttttcttcgaagtccacctgccaaagaagaaaaaatggctattctcatggatcacaactgccctattaaaacaaaaatgtacactcagaataatatccagtcatatccgataggtgaagatgaagaatctgaaagtgactga

SEQ ID NO: 5 - protein, 525 aa, Artificial Sequence
  Description of Artificial Sequence/note = synthetic construct
MLRTILDAPQRLLKEGRASRQLVLVVVFVALLLDNMLFTVVVPIVPTFLYDMEFKEVNSSLHLGHAGSSPHALASPAFSTIFSFFNNNTVAVEESVPSGIAWMNDTASTIPPPATEAISAHKNNCLQGTGFLEEEITRVGVLFASKAVMQLLVNPFVGPLTNRIGYHIPMFAGFVIMFLSTVMFAFSGTYTLLFVARTLQGIGSSFSSVAGLGMLASVYTDDHERGRAMGTALGGLALGLLVGAPFGSVMYEFVGKSAPFLILAFLALLDGALQLCILQPSKVSPESAKGTPLFMLLKDPYILVAAGSICFANMGVAILEPTLPIWMMQTMCSPKWQLGLAFLPASVSYLIGTNLFGVLANKMGRWLCSLIGMLVVGTSLLCVPLAHNIFGLIGPNAGLGLAIGMVDSSMMPIMGHLVDLRHTSVYGSVYAIADVAFCMGFAIGPSTGGAIVKAIGFPWLMVITGVINIVYAPLCYYLRSPPAKEEKLAILSQDCPMETRMYATQKPTKEFPLGEDSDEEPDHEE

SEQ ID NO: 6 - DNA, 1578 bp, Artificial Sequence
  Description of Artificial Sequence/note = synthetic construct
atgctccggaccattctggatgctccccagcggttgctgaaggaggggagagcgtcccggcagctggtgctggtggtggtattcgtcgctttgctcctggacaacatgctgtttactgtggtggtgccaattgtgcccaccttcctatatgacatggagttcaaagaagtcaactcttctctgcacctcggccatgccggaagttccccacatgccctcgcctctcctgccttttccaccatcttctccttcttcaacaacaacaccgtggctgttgaagaaagcgtacctagtggaatagcatggatgaatgacactgccagcaccatcccacctccagccactgaagccatctcagctcataaaaacaactgcttgcaaggcacaggtttcttggaggaagagattacccgggtcggggttctgtttgcttcaaaggctgtgatgcaacttctggtcaacccattcgtgggccctctcaccaacaggattggatatcatatccccatgtttgctggctttgttatcatgtttctctccacagttatgtttgctttttctgggacctatactctactctttgtggcccgaacccttcaaggcattggatcttcattttcatctgttgcaggtcttggaatgctggccagtgtctacactgatgaccatgagagaggacgagccatgggaactgctctggggggcctggccttggggttgctggtgggagctccctttggaagtgtaatgtacgagtttgttgggaagtctgcacccttcctcatcctggccttcctggcactactggatggagcactccagctttgcatcctacagccttccaaagtctctcctgagagtgccaaggggactcccctctttatgcttctcaaagacccttacatcctggtggctgcagggtccatctgctttgccaacatgggggtggccatcctggagcccacactgcccatctggatgatgcagaccatgtgctcccccaagtggcagctgggtctagctttcttgcctgccagtgtgtcctacctcattggcaccaacctctttggtgtgttggccaacaagatgggtcggtggctgtgttccctaatcgggatgctggtagtaggtaccagcttgctctgtgttcctctggctcacaatatttttggtctcattggccccaatgcagggcttggccttgccataggcatggtggattcttctatgatgcccatcatggggcacctggtggatctacgccacacctcggtgtatgggagtgtctacgccatcgctgatgtggctttttgcatgggctttgctataggtccatccaccggtggtgccattgtaaaggccatcggttttccctggctcatggtcatcactggggtcatcaacatcgtctatgctccactctgctactacctgcggagccccccggcaaaggaagagaagcttgctattctgagtcaggactgccccatggagacccggatgtatgcaacccagaagcccacgaaggaatttcctctgggggaggacagtgatgaggagcctgaccatgaggagtag

SEQ ID NO: 7 - protein, 446 aa, Artificial Sequence
  Description of Artificial Sequence/note = synthetic construct
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 8 - DNA, 1341 bp, Artificial Sequence
  Description of Artificial Sequence/note = synthetic construct
atgaggactctgaacacctctgccatggacgggactgggctggtggtggagagggacttctctgttcgtatcctcactgcctgtttcctatcgctgctcatcctgtccacgctcctggggaacacgctggtctgtgctgccgttatcaggttccgacacctgcggtccaaggtgaccaacttctttgtcatctccttggctgtgtcagatctcttggtggcagtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaataagcttttacatccctgtggccatcatgattgtcacctacaccaggatctacaggattgctcagaaacaaatacggcgcattgcggccttggagagggcagcagtccacgccaagaattgccagaccaccacaggtaatggaaagcctgtcgaatgttctcaaccggaaagttcttttaagatgtccttcaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatcacacaaaacggtcagcacccaacctga

SEQ ID NO: 9 - protein, 443 aa, Artificial Sequence
  Description of Artificial Sequence/note = synthetic construct
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 10 - DNA, 1332 bp, Artificial Sequence
  Description of Artificial Sequence/note = synthetic construct
atggatccactgaatctgtcctggtatgatgatgatctggagaggcagaactggagccggcccttcaacgggtcagacgggaaggcggacagaccccactacaactactatgccacactgctcaccctgctcatcgctgtcattgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccatcccaccacggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctga

SEQ ID NO: 11 - protein, 444 aa, Artificial Sequence
  Description of Artificial Sequence/note = synthetic construct
MDPLNLSWYDDDLERQNWSRPFNGSEGKADRPHYNYYAMLLTLLIFIIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMIAIVWVLSFTISCPLLFGLNNTDQNECIIANPAFVVYSSIVSFYVPFIVTLLVYIKIYIVLRKRRKRVNTKRSSRAFRANLKTPLKGNCTHPEDMKLCTVIMKSNGSFPVNRRRMDAARRAQELEMEMLSSTSPPERTRYSPIPPSHHQLTLPDPSHHGLHSNPDSPAKPEKNGHAKIVNPRIAKFFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFMKILHC

SEQ ID NO: 12 - DNA, 1335 bp, Artificial Sequence
  Description of Artificial Sequence/note = synthetic construct
atggatccactgaacctgtcctggtacgatgacgatctggagaggcagaactggagccggcccttcaatgggtcagaagggaaggcagacaggccccactacaactactatgccatgctgctcaccctcctcatctttatcatcgtctttggcaatgtgctggtgtgcatggctgtatcccgagagaaggctttgcagaccaccaccaactacttgatagtcagccttgctgtggctgatcttctggtggccacactggtaatgccgtgggttgtctacctggaggtggtgggtgagtggaaattcagcaggattcactgtgacatctttgtcactctggatgtcatgatgtgcacagcaagcatcctgaacctgtgtgccatcagcattgacaggtacacagctgtggcaatgcccatgctgtataacacacgctacagctccaagcgccgagttactgtcatgattgccattgtctgggtcctgtccttcaccatctcctgcccactgctcttcggactcaacaatacagaccagaatgagtgtatcattgccaaccctgcctttgtggtctactcctccattgtctcattctacgtgcccttcatcgtcactctgctggtctatatcaaaatctacatcgtcctccggaagcgccggaagcgggtcaacaccaagcgcagcagtcgagctttcagagccaacctgaagacaccactcaagggcaactgtacccaccctgaggacatgaaactctgcaccgttatcatgaagtctaatgggagtttcccagtgaacaggcggagaatggatgctgcccgccgagctcaggagctggaaatggagatgctgtcaagcaccagtcccccagagaggacccggtatagccccatccctcccagtcaccaccagctcactctccctgatccatcccaccacggcctacatagcaaccctgacagtcctgccaaaccagagaagaatgggcacgccaagattgtcaatcccaggattgccaagttctttgagatccagaccatgcccaatggcaaaacccggacctcccttaagacgatgagccgcagaaagctctcccagcagaaggagaagaaagccactcagatgcttgccattgttctcggtgtgttcatcatctgctggctgcccttcttcatcacgcacatcctgaatatacactgtgattgcaacatcccaccagtcctctacagcgccttcacatggctgggctatgtcaacagtgccgtcaaccccatcatctacaccaccttcaacatcgagttccgcaaggccttcatgaagatcttgcactgctga

SEQ ID NO: 13 - protein, 444 aa, Artificial Sequence; identical to SEQ ID NO: 11
  Description of Artificial Sequence/note = synthetic construct

SEQ ID NO: 14 - protein, 619 aa, Artificial Sequence
  Description of Artificial Sequence/note = synthetic construct
MSKSKCSVGPMSSVVAPAKEPNAVGPREVELILVKEQNGVQLTNSTLINPPQTPVEVQERETWSKKIDFLLSVIGFAVDLANVWRFPYLCYKNGGGAFLVPYLLFMVIAGMPLFYMELALGQFNREGAAGVWKICPVLKGVGFTVILISFYVGFFYNVIIAWALHYFFSSFTMDLPWIHCNNTWNSPNCSDAHSSNSSDGLGLNDTFGTTPAAEYFERGVLHLHQSRGIDDLGPPRWQLTACLVLVIVLLYFSLWKGVKTSGKVVWITATMPYVVLTALLLRGVTLPGAMDGIRAYLSVDFYRLCEASVWIDAATQVCFSLGVGFGVLIAFSSYNKFTNNCYRDAIITTSINSLTSFSSGFVVFSFLGYMAQKHNVPIRDVATDGPGLIFIIYPEAIATLPLSSAWAAVFFLMLLTLGIDSAMGGMESVITGLVDEFQLLHRHRELFTLGIVLATFLLSLFCVTNGGIYVFTLLDHFAAGTSILFGVLIEAIGVAWFYGVQQFSDDIKQMTGQRPNLYWRLCWKLVSPCFLLYVVVVSIVTFRPPHYGAYIFPDWANALGWIIATSSMAMVPIYATYKFCSLPGSFREKLAYAITPEKDRQLVDRGEVRQFTLRHWLLV

SEQ ID NO: 15 - protein, 619 aa, Artificial Sequence
  Description of Artificial Sequence/note = synthetic construct
MSKSKCSVGPMSSVVAPAKESNAVGPREVELILVKEQNGVQLTNSTLINPPQTPVEAQERETWSKKIDFLLSVIGFAVDLANVWRFPYLCYKNGGGAFLVPYLLFMVIAGMPLFYMELALGQFNREGAAGVWKICPVLKGVGFTVILISFYVGFFYNVIIAWALHYFFSSFTMDLPWIHCNNTWNSPNCSDAHASNSSDGLGLNDTFGTTPAAEYFERGVLHLHQSRGIDDLGPPRWQLTACLVLVIVLLYFSLWKGVKTSGKVVWITATMPYVVLTALLLRGVTLPGAMDGIRAYLSVDFYRLCEASVWIDAATQVCFSLGVGFGVLIAFSSYNKFTNNCYRDAIITTSINSLTSFSSGFVVFSFLGYMAQKHNVPIRDVATDGPGLIFIIYPEAIATLPLSSAWAAVFFLMLLTLGIDSAMGGMESVITGLVDEFQLLHRHRELFTLGIVLATFLLSLFCVTNGGIYVFTLLDHFAAGTSILFGVLIEAIGVAWFYGVQQFSDDIKQMTGQRPNLYWRLCWKLVSPCFLLYVVVVSIVTFRPPHYGAYIFPDWANALGWIIATSSMAMVPIYATYKFCSLPGSFREKLAYAITPEKDHQLVDRGEVRQFTLRHWLLL

SEQ ID NO: 16 - protein, 620 aa, Artificial Sequence
  Description of Artificial Sequence/note = synthetic construct
MSKSKCSVGLMSSVVAPAKEPNAVGPKEVELILVKEQNGVQLTSSTLTNPRQSPVEAQDRETWGKKIDFLLSVIGFAVDLANVWRFPYLCYKNGGGAFLVPYLLFMVIAGMPLFYMELALGQFNREGAAGVWKICPILKGVGFTVILISLYVGFFYNVIIAWALHYLFSSFTTELPWIHCNNSWNSPNCSDAHPGDSSGDSSGLNDTFGTTPAAEYFERGVLHLHQSHGIDDLGPPRWQLTACLVLVIVLLYFSLWKGVKTSGKVVWITATMPYVVLTALLLRGVTLPGAIDGIRAYLSVDFYRLCEASVWIDAATQVCFSLGVGFGVLIAFSSYNKFTNNCYRDAIVTTSINSLTSFSSGFVVFSFLGYMAQKHSVPIGDVAKDGPGLIFIIYPEAIATLPLSSAWAVVFFIMLLTLGIDSAMGGMESVITGLIDEFQLLHRHRELFTLFIVLATFLLSLFCVTNGGIYVFTLLDHFAAGTSILFGVLIEAIGVAWFYGVGQFSDDIQQMTGQRPSLYWRLCWKLVSPCFLLFVVVVSIVTFRPPHYGAYIFPDWANALGWVIATSSMAMVPIYAAYKFCSLPGSFREKLAYAIAPEKDRELVDRGEVRQFTLRHWLKV

SEQ ID NO: 17 - DNA, 1873 bp, Artificial Sequence
  Description of Artificial Sequence/note = synthetic construct
tacccatgagtaaaagcaaatgctccgtgggaccaatgtcttctgtggtggccccggctaaagagcccaatgctgtgggccccagagaggtggagctcatcttggtcaaggagcagaatggagtgcagctgaccaattccaccctcatcaacccaccgcagacaccagtggaggttcaagagcgggagacctggagcaagaaaatcgatttcctgctctcagtcatcggcttcgctgtggacctggccaatgtttggaggtttccctacctgtgctacaaaaatggtggaggtgccttcctggtgccctacctgctcttcatggttattgccgggatgcccctcttctacatggagctggctctcgggcagttcaacagagaaggagctgctggtgtctggaagatctgccctgtcctgaaaggtgtgggcttcactgtcatcctcatctctttctacgtgggcttcttctacaatgtcatcattgcatgggcactgcactacttcttctcctccttcaccatggacctcccatggatccactgcaacaacacctggaacagccccaactgttctgatgcacatagcagcaactctagcgatggcctgggcctcaacgacacctttgggaccacacccgctgctgagtattttgagcgtggtgtgctgcacctccatcagagtcgtggcattgatgacctgggccctccacggtggcagctcacagcctgcctggtgctggtcattgttctgctctacttcagcctgtggaagggagtaaagacttcagggaaggtggtgtggatcacagctaccatgccctatgtagtcctcacagccctgctcctgcgtggagtcaccctccctggggccatggatggcatcagagcatacctcagtgtggacttctaccgtctctgtgaggcatctgtgtggatcgatgccgccacccaggtgtgcttctcccttggcgttgggtttggggtgctgattgccttctccagttacaataagttcaccaataactgctatagagatgcaatcatcaccacctccattaactccctgacgagcttctcctctggcttcgttgtcttctccttcctggggtacatggcacagaagcacaatgtgcccatcagggatgtggccacagatggacctgggttgatcttcatcatctaccctgaggcaatcgccacactcccgctgtcttcagcctgggccgctgtcttcttcctcatgctgctcactctgggtatcgacagtgccatggggggcatggagtctgtgatcactgggcttgtcgatgagttccagctgctacatcggcatcgagagctcttcactcttggcattgtcctggctactttcctgctgtctctcttctgtgtcaccaacggtggcatctatgtcttcacactgctggaccactttgcagctggcacatctatcctctttggagtgctcattgaagccattggggtggcctggttctacggtgtccagcaattcagtgatgacatcaagcagatgactgggcagcgacccaacctgtactggcggctatgctggaagctggtcagcccctgcttccttctgtatgtggtcgtggtcagcattgtgaccttcagacccccacactatggagcctacatcttcccagactgggccaatgccctgggctggatcattgccacatcctccatggccatggtgcccatttatgccacctataagttctgcagcctgccagggtccttccgagagaaactggcctatgccatcacacctgagaaagaccgccagctagtggacagaggggaggtgcgccaattcacgctgcgccattggctgttgctgtaaagtggaag

SEQ ID NO: 18 - DNA, 2020 bp, Artificial Sequence
  Description of Artificial Sequence/note = synthetic construct
gaattcctcaactcccagtgtgcccatgagtaagagcaaatgctccgtgggactcatgtcttccgtggtggccccggctaaggagcccaatgccgtgggcccgaaggaggtggagctcatccttgtcatggagcagaacggagtgcagctcaccagctccaccctcaccaacccgcggcagagccccgtggaggcccaggatcgggagacctggggcaagaagatcgactttctcctgtccgtcattggctttgctgtggacctggccaacgtctggaggttcccctacctgtgctacaaaaatggtggcggtgccttcctggtcccctacctgctcttcatggtcattgctgggatgccacttttctacatggagctggccctcggccagttcaacagggaaggggccgctggtgtctggaagatctgccccatactgaaaggtgtgggcttcacggtcatcctcatctcactgtatgtcggcttcttctacaacgtcatcatcgcctgggcgctgcactatctcttctcctccttcaccacggagctcccctggatccactgcaacaactcctggaacagccccaactgctcggatgcccatcctggtgactccagtggagacagctcgggcctcaacgacacttttgggaccacacctgctgccgagtactttgaacgtggcgtgctgcacctccaccagagccatggcatcgacgacctggggcctccgcggtggcagctcacagcctgcctggtgctggtcatcgtgctgctgtacttcagcctctggaagggcgtgaagacctcagggaaggtggtatggatcacagccaccatgccatacgtggtcctcactgccctgctcctgcgtggggtcaccctccctggagccatagacggcatcagagcatacctgagcgttgacttctaccggctctgcgaggcgtctgtttggattgacgcggccacccaggtgtgcttctccctgggcgtggggttcggggtgctgatcgccttctccagctacaacaagttcaccaacaactgctacagggacgcgattgtcaccacctccatcaactgcctgacgagcttctcctccggcttcgtcgtcttctccttcctggggtacatggcacagaagcacagtgtgcccatcggggacgtggccaaggacgggccagggctgatcttcatcatctacccggaagccatcgccacgctccctctgtcctcggcctgggcggtggtcttcttcatcatgctgctcaccctgggtatcgacagcgccatgggtggtatggagtcagtgatcaccgggctcatcgatgagttccagctgctgcacagacaccgtgagctcttcacgctcttcatcgtcctggcgaccttcctcctgtccctgttctgcgtcaccaacggtggcatctacgtcttcacgctcctggaccattttgcagccggcacgtccatcctctttggagtgctcatcgaagccatcggagtggcctggttctatggtgttgggcagttcagcgacgacatccagcagatgaccgggcagcggcccagcctgtactggcggctgtgctggaagctggtcagcccctgctttctcctgttcgtggtcgtggtcagcattgtgaccttcagacccccccactacggagcctacatcttccccgactgggccaacgcgctgggctgggtcatcgccacatcctccatggccatggtgcccatctatgcggcctacaagttctgcagcctgcctgggtcctttcgagagaaactggcctacgccattgcacccgagaaggaccgtgagctggtggacagaggggaggtgcgccagttcacgctccgccactggctcaaggtgtagagggagcagagacgaagaccccaggaagtcatcccgcaatgggagagacacgaacaaaccaaggaaatctaagtttcgagagaaaggaggggcaacttctactcttcaacctcttactgaaaacacaaacca